SEQUENCE LISTING (211 sequences)

SEQ ID NO: 1 - protein, 2416 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACLRTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATLATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAATEESTEGPSATEVPSASEEPSPSEVPFPSEEPSPSEEPFPSVRPFPSVELFPSEEPFPSKEPSPSEEPSASEEPYTPSPPVPSWTELPSSGEESGAPDVSGDFTGSGDVSGHLDFSGQLSGDRASGLPSGDLDSSGLTSTVGSGLPVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGLPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGISGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAAAPARSCAEEPCGAGTCKETEGHVICLCPPGYTGEHCNIDQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTVACGEPPVVEHARTFGQKKDRYEINSLVRYQCTEGFVQRHMPTIRCQPSGHWEEPQITCTDPTTYKRRLQKRSSRHPRRSRPSTAH

SEQ ID NO: 2 - protein, 380 aa, Homo sapiens
MKEYVLLLFLALCSAKPFFSPSHIALKNMMLKDMEDTDDDDDDDDDDDDDDEDNSLFPTREPRSHFFPFDLFPMCPFGCQCYSRVVHCSDLGLTSVPTNIPFDTRMLDLQNNKIKEIKENDFKGLTSLYGLILNNNKLTKIHPKAFLTTKKLRRLYLSHNQLSEIPLNLPKSLAELRIHENKVKKIQKDTFKGMNALHVLEMSANPLDNNGIEPGAFEGVTVFHIRIAEAKLTSVPKGLPPTLLELHLDYNKISTVELEDFKRYKELQRLGLGNNKITDIENGSLANIPRVREIHLENNKLKKIPSGLPELKYLQIIFLHSNSIARVGVNDFCPTVPKMKKSLYSAISLFNNPVKYWEMQPATFRCVLSRMSVQLGNFGM

SEQ ID NO: 3 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 4 - protein, 100 aa, Homo sapiens
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 5 - protein, 369 aa, Homo sapiens
MLSVCVAFLFLCTAHLPIYSSALPFEQKGFWDFGKDIDVKELMMMMDQEEGSAVEPYKPEHPTCPFGCRCELRVVQCSDLGLGYVPYDIPADTLLLDLQSNRITEIREGDFKGLSNLYALVLRYNQISKIHPKAFLPLKRLQKLYISHNLLTSMPKNLPSSLVELRIHDNRIKKVPAFSFSGLHNMHVIEMGRNPLQNSGFEPGAFMGLKLNYLRISEAKLTGVPKDLPGSLHELHLDNNQIQAIELVDLSQYTQLQRLGLGSNQIRHIEHGALSYLTNLRELHLDNNRLPSVPSGLSHMKYLQVVYLHSNNITNVGEDDFCPTGFGLKKVFYNGISLFDNPIRYWEVQPSTFRCVSDQMAVQFGNHKK

SEQ ID NO: 6 - protein, 675 aa, Homo sapiens
MDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIWELKREEITLLKELGSGQFGVVQLGKWKGQYDVAVKMIKEGSMSEDEFFQEAQTMMKLSHPKLVKFYGVCSKEYPIYIVTEYISNGCLLNYLRSHGKGLEPSQLLEMCYDVCEGMAFLESHQFIHRDLAARNCLVDRDLCVKVSDFGMTRYVLDDQYVSSVGTKFPVKWSAPEVFHYFKYSSKSDVWAFGILMWEVFSLGKQPYDLYDNSQVVLKVSQGHRLYRPHLASDTIYQIMYSCWHELPEKRPTFQQLLSSIEPLREKDKH

SEQ ID NO: 7 - protein, 138 aa, Homo sapiens
MTWRQAVLLSCFSAVVLLSMLREGTSVSVGTMQMAGEEASEDAKQKIFMQESDASNFLKRRGKRSPKSRDEVNVENRQKLRVDELRREYYEEQRNEFENFVEEQNDEQEERSREAVEQWRQWHYDGLHPSYLYNRHHT

SEQ ID NO: 8 - protein, 315 aa, Homo sapiens
MDLRQFLMCLSLCTAFALSKPTEKKDRVHHEPQLSDKVHNDAQSFDYDHDAFLGAEEAKTFDQLTPEESKERLGKIVSKIDGDKDGFVTVDELKDWIKFAQKRWIYEDVERQWKGHDLNEDGLVSWEEYKNATYGYVLDDPDPDDGFNYKQMMVRDERRFKMADKDGDLIATKEEFTAFLHPEEYDYMKDIVVQETMEDIDKNADGFIDLEEYIGDMYSHDGNTDEPEWVKTEREQFVEFRDKNRDGKMDKEETKDWILPSDYDHAEAEARHLVYESDQNKDGKLTKEEIVDKYDLFVGSQATDFGEALVRHDEF

SEQ ID NO: 9 - protein, 326 aa, Homo sapiens
MEFLSEKFALKSPPSKNSDFYMGAGGPLEHVMETLDNESFYSKASAGKCVQAFGPLPRAEHHVRLERTSPCQDSSVNYGITKVEGQPLHTELNRAMDNCNSLRMSPVKGMQEKGELDELGDKCDSNVSSSKKRRHRTTFTSLQLEELEKVFQKTHYPDVYVREQLALRTELTEARVQVWFQNRRAKWRKRERYGQIQQAKSHFAATYDISVLPRTDSYPQIQNNLWAGNASGGSVVTSCMLPRDTSSCMTPYSHSPRTDSSYTGFSNHQNQFSHVPLNNFFTDSLLTGATNGHAFETKPEFERRSSSIAVLRMKAKEHTANISWAM

SEQ ID NO: 10 - protein, 359 aa, Homo sapiens
MVRPMLLLSLGLLAGLLPALAACPQNCHCHSDLQHVICDKVGLQKIPKVSEKTKLLNLQRNNFPVLAANSFRAMPNLVSLHLQHCQIREVAAGAFRGLKQLIYLYLSHNDIRVLRAGAFDDLTELTYLYLDHNKVTELPRGLLSPLVNLFILQLNNNKIRELRAGAFQGAKDLRWLYLSENALSSLQPGALDDVENLAKFHVDRNQLSSYPSAALSKLRVVEELKLSHNPLKSIPDNAFQSFGRYLETLWLDNTNLEKFSDGAFLGVTTLKHVHLENNRLNQLPSNFPFDSLETLALTNNPWKCTCQLRGLRRWLEAKASRPDATCASPAKFKGQHIRDTDAFRSCKFPTKRSKKAGRH

SEQ ID NO: 11 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 12 - protein, 1184 aa, Homo sapiens
MVGTKAWVFSFLVLEVTSVLGRQTMLTQSVRRVQPGKKNPSIFAKPADTLESPGEWTTWFNIDYPGGKGDYERLDAIRFYYGDRVCARPLRLEARTTDWTPAGSTGQVVHGSPREGFWCLNREQRPGQNCSNYTVRFLCPPGSLRRDTERIWSPWSPWSKCSAACGQTGVQTRTRICLAEMVSLCSEASEEGQHCMGQDCTACDLTCPMGQVNADCDACMCQDFMLHGAVSLPGGAPASGAAIYLLTKTPKLLTQTDSDGRFRIPGLCPDGKSILKITKVKFAPIVLTMPKTSLKAATIKAEFVRAETPYMVMNPETKARRAGQSVSLCCKATGKPRPDKYFWYHNDTLLDPSLYKHESKLVLRKLQQHQAGEYFCKAQSDAGAVKSKVAQLIVIASDETPCNPVPESYLIRLPHDCFQNATNSFYYDVGRCPVKTCAGQQDNGIRCRDAVQNCCGISKTEEREIQCSGYTLPTKVAKECSCQRCTETRSIVRGRVSAADNGEPMRFGHVYMGNSRVSMTGYKGTFTLHVPQDTERLVLTFVDRLQKFVNTTKVLPFNKKGSAVFHEIKMLRRKEPITLEAMETNIIPLGEVVGEDPMAELEIPSRSFYRQNGEPYIGKVKASVTFLDPRNISTATAAQTDLNFINDEGDTFPLRTYGMFSVDFRDEVTSEPLNAGKVKVHLDSTQVKMPEHISTVKLWSLNPDTGLWEEEGDFKFENQRRNKREDRTFLVGNLEIRERRLFNLDVPESRRCFVKVRAYRSERFLPSEQIQGVVISVINLEPRTGFLSNPRAWGRFDSVITGPNGACVPAFCDDQSPDAYSAYVLASLAGEELQAVESSPKFNPNAIGVPQPYLNKLNYRRTDHEDPRVKKTAFQISMAKPRPNSAEESNGPIYAFENLRACEEAPPSAAHFRFYQIEGDRYDYNTVPFNEDDPMSWTEDYLAWWPKPMEFRACYIKVKIVGPLEVNVRSRNMGGTHRRTVGKLYGIRDVRSTRDRDQPNVSAACLEFKCSGMLYDQDRVDRTLVKVIPQGSCRRASVNPMLHEYLVNHLPLAVNNDTSEYTMLAPLDPLGHNYGIYTVTDQDPRTAKEIALGRCFDGTSDGSSRIMKSNVGVALTFNCVERQVGRQSAFQYLQSTPAQSPAAGTVQGRVPSRRQQRASRGGQRQGGVVASLRFPRVAQQPLIN

SEQ ID NO: 13 - protein, 1156 aa, Homo sapiens
MASLLPLLCLCVVAAHLAGARDATPTEEPMATALGLERRSVYTGQPSPALEDWEEASEWTSWFNVDHPGGDGDFESLAAIRFYYGPARVCPRPLALEARTTDWALPSAVGERVHLNPTRGFWCLNREQPRGRRCSNYHVRFRCPLEASWGAWGPWGPCSGSCGPGRRLRRRHCPSPAGDACPGRPLEAQKCVRPRCPGCSLDTCECPDHILLGSVVTPSGQPLLGARVSLRDQPGTVATSDAHGTFRVPGVCADSRANIRAQMDGFSAGEAQAQANGSISVVTIILDKLEKPYLVKHPESRVREAGQNVTFCCKASGTPMPKKYSWFHNGTLLDRRAHGYGAHLELRGLRPDQAGIYHCKAWNEAGAVRSGTARLTVLAPGQPACDPRPREYLIKLPEDCGQPGSGPAYLDVGLCPDTRCPSLAGSSPRCGDASSRCCSVRRLERREIHCPGYVLPVKVVAECGCQKCLPPRGLVRGRVVAADSGEPLRFARILLGQEPIGFTAYQGDFTIEVPPSTQRLVVTFVDPSGEFMDAVRVLPFDPRGAGVYHEVKAMRKKAPVILHTSQSNTIPLGELEDEAPLGELVLPSGAFRRADGKPYSGPVEARVTFVDPRDLTSAASAPSDLRFVDSDGELAPLRTYGMFSVDLRAPGSAEQLQVGPVAVRVAASQIHMPGHVEALKLWSLNPETGLWEEESGFRREGSSGPRVRREERVFLVGNVEIRERRLFNLDVPERRRCFVKVRAYANDKFTPSEQVEGVVVTLVNLEPAPGFSANPRAWGRFDSAVTGPNGACLPAFCDADRPDAYTALVTATLGGEELEPAPSLPRPLPATVGVTQPYLDRLGYRRTDHDDPAFKRNGFRINLAKPRPGDPAEANGPVYPWRSLRECQGAPVTASHFRFARVEADKYEYNVVPFREGTPASWTGDLLAWWPNPQEFRACFLKVKIQGPQEYMVRSHNAGGSHPRTRGQLYGLRDARSVRDPERPGTSAACVEFKCSGMLFDQRQVDRTLVTIMPQGSCRRVAVNGLLRDYLTRHPPPVPAEDPAAFSMLAPLDPLGHNYGVYTVTDQSPRLAKEIAIGRCFDGSSDGFSREMKADAGTAVTFQCREPPAGRPSLFQRLLESPATALGDIRREMSEAAQAQARASGPLRTRRGRVRQ

SEQ ID NO: 14 - protein, 197 aa, Homo sapiens
MAKNGLVICILVITLLLDQTTSHTSRLKARKHSKRRVRDKDGDLKTQIEKLWTEVNALKEIQALQTVCLRGTKVHKKCYLASEGLKHFHEANEDCISKGGILVIPRNSDEINALQDYGKRSLPGVNDFWLGINDMVTEGKFVDVNGIAISFLNWDRAQPNGGKRENCVLFSQSAQGKWSDEACRSSKRYICEFTIPQ

SEQ ID NO: 15 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 16 - protein, 661 aa, Homo sapiens
MAPSADPGMSRMLPFLLLLWFLPITEGSQRAEPMFTAVTNSVLPPDYDSNPTQLNYGVAVTDVDHDGDFEIVVAGYNGPNLVLKYDRAQKRLVNIAVDERSSPYYALRDRQGNAIGVTACDIDGDGREEIYFLNTNNAFSGVATYTDKLFKFRNNRWEDILSDEVNVARGVASLFAGRSVACVDRKGSGRYSIYIANYAYGNVGPDALIEMDPEASDLSRGILALRDVAAEAGVSKYTGGRGVSVGPILSSSASDIFCDNENGPNFLFHNRGDGTFVDAAASAGVDDPHQHGRGVALADFNRDGKVDIVYGNWNGPHRLYLQMSTHGKVRFRDIASPKFSMPSPVRTVITADFDNDQELEIFFNNIAYRSSSANRLFRVIRREHGDPLIEELNPGDALEPEGRGTGGVVTDFDGDGMLDLILSHGESMAQPLSVFRGNQGFNNNWLRVVPRTRFGAFARGAKVVLYTKKSGAHLRIIDGGSGYLCEMEPVAHFGLGKDEASSVEVTWPDGKMVSRNVASGEMNSVLEILYPRDEDTLQDPAPLECGQGFSQQENGHCMDTNECIQFPFVCPRDKPVCVNTYGSYRCRTNKKCSRGYEPNEDGTACVGTLGQSPGPRPTTPTAAAATAAAAAAAGAATAAPVLVDGDLNLGSVVKESCEPSC

SEQ ID NO: 17 - protein, 136 aa, Homo sapiens
MRTPGPLPVLLLLLAGAPAARPTPPTCYSRMRALSQEITRDFNLLQVSEPSEPCVRYLPRLYLDIHNYCVLDKLRDFVASPPCWKVAQVDSLKDKARKLYTIMNSFCRRDLVFLLDDCNALEYPIPVTTVLPDRQR

SEQ ID NO: 18 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 19 - protein, 322 aa, Homo sapiens
MKTLAGLVLGLVIFDAAVTAPTLESINYDSETYDATLEDLDNLYNYENIPVDKVEIEIATVMPSGNRELLTPPPQPEKAQEEEEEEESTPRLIDGSSPQEPEFTGVLGPHTNEDFPTCLLCTCISTTVYCDDHELDAIPPLPKNTAYFYSRFNRIKKINKNDFASLSDLKRIDLTSNLISEIDEDAFRKLPQLRELVLRDNKIRQLPELPTTLTFIDISNNRLGRKGIKQEAFKDMYDLHHLYLTDNNLDHIPLPLPENLRALHLQNNNILEMHEDTFCNVKNLTYIRKALEDIRLDGNPINLSKTPQAYMCLPRLPVGSLV

SEQ ID NO: 20 - protein, 452 aa, Homo sapiens
MLCGRPRSSSDNRNFLRERAGLSSAAVQTRIGNSAASRRSPAARPPVPAPPALPRGRPGTEGSTSLSAPAVLVVAVAVVVVVVSAVAWAMANYIHVPPGSPEVPKLNVTVQDQEEHRCREGALSLLQHLRPHWDPQEVTLQLFTDGITNKLIGCYVGNTMEDVVLVRIYGNKTELLVDRDEEVKSFRVLQAHGCAPQLYCTFNNGLCYEFIQGEALDPKHVCNPAIFRLIARQLAKIHAIHAHNGWIPKSNLWLKMGKYFSLIPTGFADEDINKRFLSDIPSSQILQEEMTWMKEILSNLGSPVVLCHNDLLCKNIIYNEKQGDVQFIDYEYSGYNYLAYDIGNHFNEFAGVSDVDYSLYPDRELQSQWLRAYLEAYKEFKGFGTEVTEKEVEILFIQVNQFALASHFFWGLWALIQAKYSTIEFDFLGYAIVRFNQYFKMKPEVTALKVPE

SEQ ID NO: 21 - protein, 660 aa, Homo sapiens
MGLQTTKWPSHGAFFLKSWLIISLGLYSQVSKLLACPSVCRCDRNFVYCNERSLTSVPLGIPEGVTVLYLHNNQINNAGFPAELHNVQSVHTVYLYGNQLDEFPMNLPKNVRVLHLQENNIQTISRAALAQLLKLEELHLDDNSISTVGVEDGAFREAISLKLLFLSKNHLSSVPVGLPVDLQELRVDENRIAVISDMAFQNLTSLERLIVDGNLLTNKGIAEGTFSHLTKLKEFSIVRNSLSHPPPDLPGTHLIRLYLQDNQINHIPLTAFSNLRKLERLDISNNQLRMLTQGVFDNLSNLKQLTARNNPWFCDCSIKWVTEWLKYIPSSLNVRGFMCQGPEQVRGMAVRELNMNLLSCPTTTPGLPLFTPAPSTASPTTQPPTLSIPNPSRSYTPPTPTTSKLPTIPDWDGRERVTPPISERIQLSIHFVNDTSIQVSWLSLFTVMAYKLTWVKMGHSLVGGIVQERIVSGEKQHLSLVNLEPRSTYRICLVPLDAFNYRAVEDTICSEATTHASYLNNGSNTASSHEQTTSHSMGSPFLLAGLIGGAVIFVLVVLLSVFCWHMHKKGRYTSQKWKYNRGRRKDDYCEAGTKKDNSILEMTETSFQIVSLNNDQLLKGDFRLQPIYTPNGGINYTDCHIPNNMRYCNSSVPDLEHCHT

SEQ ID NO: 22 - protein, 649 aa, Homo sapiens
MISAAWSIFLIGTKIGLFLQVAPLSVMAKSCPSVCRCDAGFIYCNDRFLTSIPTGIPEDATTLYLQNNQINNAGIPSDLKNLLKVERIYLYHNSLDEFPTNLPKYVKELHLQENNIRTITYDSLSKIPYLEELHLDDNSVSAVSIEEGAFRDSNYLRLLFLSRNHLSTIPWGLPRTIEELRLDDNRISTISSPSLQGLTSLKRLVLDGNLLNNHGLGDKVFFNLVNLTELSLVRNSLTAAPVNLPGTNLRKLYLQDNHINRVPPNAFSYLRQLYRLDMSNNNLSNLPQGIFDDLDNITQLILRNNPWYCGCKMKWVRDWLQSLPVKVNVRGLMCQAPEKVRGMAIKDLNAELFDCKDSGIVSTIQITTAIPNTVYPAQGQWPAPVTKQPDIKNPKLTKDHQTTGSPSRKTITITVKSVTSDTIHISWKLALPMTALRLSWLKLGHSPAFGSITETIVTGERSEYLVTALEPDSPYKVCMVPMETSNLYLFDETPVCIETETAPLRMYNPTTTLNREQEKEPYKNPNLPLAAIIGGAVALVTIALLALVCWYVHRNGSLFSRNCAYSKGRRRKDDYAEAGTKKDNSILEIRETSFQMLPISNEPISKEEFVIHTIFPPNGMNLYKNNHSESSSNRSYRDSGIPDSDHSHS

SEQ ID NO: 23 - protein, 354 aa, Homo sapiens
MKSLLLLVLISICWADHLSDNYTLDHDRAIHIQAENGPHLLVEAEQAKVFSHRGGNVTLPCKFYRDPTAFGSGIHKIRIKWTKLTSDYLKEVDVFVSMGYHKKTYGGYQGRVFLKGGSDSDASLVITDLTLEDYGRYKCEVIEGLEDDTVVVALDLQGVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

SEQ ID NO: 24 - protein, 125 aa, Homo sapiens
MRPRCCILALVCWITVFLLQCSKGTTDAPVGSGLWLCQPTPRCGNKIYNPSEQCCYDDAILSLKETRRCGSTCTFWPCFELCCPESFGPQQKFLVKLRVLGMKSQCHLSPISRSCTRNRRHVLYP

SEQ ID NO: 25 - protein, 1263 aa, Homo sapiens
MPARIGYYEIDRTIGKGNFAVVKRATHLVTKAKVAIKIIDKTQLDEENLKKIFREVQIMKMLCHPHIIRLYQVMETERMIYLVTEYASGGEIFDHLVAHGRMAEKEARRKFKQIVTAVYFCHCRNIVHRDLKAENLLLDANLNIKIADFGFSNLFTPGQLLKTWCGSPPYAAPELFEGKEYDGPKVDIWSLGVVLYVLVCGALPFDGSTLQNLRARVLSGKFRIPFFMSTECEHLIRHMLVLDPNKRLSMEQICKHKWMKLGDADPNFDRLIAECQQLKEERQVDPLNEDVLLAMEDMGLDKEQTLQSLRSDAYDHYSAIYSLLCDRHKRHKTLRLGALPSMPRALAFQAPVNIQAEQAGTAMNISVPQVQLINPENQIVEPDGTLNLDSDEGEEPSPEALVRYLSMRRHTVGVADPRTEVMEDLQKLLPGFPGVNPQAPFLQVAPNVNFMHNLLPMQNLQPTGQLEYKEQSLLQPPTLQLLNGMGPLGRRASDGGANIQLHAQQLLKRPRGPSPLVTMTPAVPAVTPVDEESSDGEPDQEAVQSSTYKDSNTLHLPTERFSPVRRFSDGAASIQAFKAHLEKMGNNSSIKQLQQECEQLQKMYGGQIDERTLEKTQQQHMLYQQEQHHQILQQQIQDSICPPQPSPPLQAACENQPALLTHQLQRLRIQPSSPPPNHPNNHLFRQPSNSPPPMSSAMIQPHGAASSSQFQGLPSRSAIFQQQPENCSSPPNVALTCLGMQQPAQSQQVTIQVQEPVDMLSNMPGTAAGSSGRGISISPSAGQMQMQHRTNLMATLSYGHRPLSKQLSADSAEAHSLNVNRFSPANYDQAHLHPHLFSDQSRGSPSSYSPSTGVGFSPTQALKVPPLDQFPTFPPSAHQQPPHYTTSALQQALLSPTPPDYTRHQQVPHILQGLLSPRHSLTGHSDIRLPPTEFAQLIKRQQQQRQQQQQQQQQQEYQELFRHMNQGDAGSLAPSLGGQSMTERQALSYQNADSYHHHTSPQHLLQIRAQECVSQASSPTPPHGYAHQPALMHSESMEEDCSCEGAKDGFQDSKSSSTLTKGCHDSPLLLSTGGPGDPESLLGTVSHAQELGIHPYGHQPTAAFSKNKVPSREPVIGNCMDRSSPGQAVELPDHNGLGYPARPSVHEHHRPRALQRHHTIQNSDDAYVQLDNLPGMSLVAGKALSSARMSDAVLSQSSLMGSQQFQDGENEECGASLGGHEHPDLSDGSQHLNSSCYPSTCITDILLSYKHPEVSFSMEQAGV

SEQ ID NO: 26 - protein, 356 aa, Homo sapiens
MGLMVRGGVTCRSQERLLGVGTGTASAQPPVTASPYCSFTGRSRADAPARAAGNGQPRLRAQVDLSSASSVLVTVLDAKALPLTLLSWGSLCATAQNHNNQEGAPAQPKGSMANQRALRFPPPSGSPPGLHTRMRLTSPPQPHFEPPPPTTVGAPRSLQGDWFGSDSRAVRALRLIGWASRSLHPLPGSRDRAHPAAEEEDDPDRPIEFSSSKANPHRWSVGHTMGKGHQRPWWKVLPLSCFLVALIIWCYLREESEADQWLRQEGSRQPPFGFDVTFARDCPGYACVLSTEGLGWWMGHLAMLIRVKAEQNLSRSETAPRLALDVQGFHRQDFSDPWGRFQLHCMLLDLPSLCIT

SEQ ID NO: 27 - protein, 75 aa, Homo sapiens
MSHIQLLSAFESHRTTCRKNSREFGKKGGEDRQKMHFSFKKQENVRKIEGVLKCVCCCLTQVNPVAGHSALLIAS

SEQ ID NO: 28 - protein, 237 aa, Homo sapiens
MKGILVAGITAVLVAAVESLSCVQCNSWEKSCVNSIASECPSHANTSCISSSASSSLETPVRLYQNMFCSAENCSEETHITAFTVHVSAEEHFHFVSQCCQGKECSNTSDALDPPLKNVSSNAECPACYESNGTSCRGKPWKCYEEEQCVFLVAELKNDIESKSLVLKGCSNVSNATCQFLSGENKTLGGVIFRKFECANVNSLTPTSAPTTSHNVGSKASLYLLALASLLLRGLLP

SEQ ID NO: 29 - protein, 753 aa, Homo sapiens
MRPVSVWQWSPWGLLLCLLCSSCLGSPSPSTGPEKKAGSQGLRFRLAGFPRKPYEGRVEIQRAGEWGTICDDDFTLQAAHILCRELGFTEATGWTHSAKYGPGTGRIWLDNLSCSGTEQSVTECASRGWGNSDCTHDEDAGVICKDQRLPGFSDSNVIEVEHHLQVEEVRIRPAVGWGRRPLPVTEGLVEVRLPDGWSQVCDKGWSAHNSHVVCGMLGFPSEKRVNAAFYRLLAQRQQHSFGLHGVACVGTEAHLSLCSLEFYRANDTARCPGGGPAVVSCVPGPVYAASSGQKKQQQSKPQGEARVRLKGGAHPGEGRVEVLKASTWGTVCDRKWDLHAASVVCRELGFGSAREALSGARMGQGMGAIHLSEVRCSGQELSLWKCPHKNITAEDCSHSQDAGVRCNLPYTGAETRIRLSGGRSQHEGRVEVQIGGPGPLRWGLICGDDWGTLEAMVACRQLGLGYANHGLQETWYWDSGNITEVVMSGVRCTGTELSLDQCAHHGTHITCKRTGTRFTAGVICSETASDLLLHSALVQETAYIEDRPLHMLYCAAEENCLASSARSANWPYGHRRLLRFSSQIHNLGRADFRPKAGRHSWVWHECHGHYHSMDIFTHYDILTPNGTKVAEGHKASFCLEDTECQEDVSKRYECANFGEQGITVGCWDLYRHDIDCQWIDITDVKPGNYILQVVINPNFEVAESDFTNNAMKCNCKYDGHRIWVHNCHIGDAFSEEANRRFERYPGQTSNQII

SEQ ID NO: 30 - protein, 756 aa, Homo sapiens
MAWSPPATLFLFLLLLGQPPPSRPQSLGTTKLRLVGPESKPEEGRLEVLHQGQWGTVCDDNFAIQEATVACRQLGFEAALTWAHSAKYGQGEGPIWLDNVRCVGTESSLDQCGSNGWGVSDCSHSEDVGVICHPRRHRGYLSETVSNALGPQGRRLEEVRLKPILASAKQHSPVTEGAVEVKYEGHWRQVCDQGWTMNNSRVVCGMLGFPSEVPVDSHYYRKVWDLKMRDPKSRLKSLTNKNSFWIHQVTCLGTEPHMANCQVQVAPARGKLRPACPGGMHAVVSCVAGPHFRPPKTKPQRKGSWAEEPRVRLRSGAQVGEGRVEVLMNRQWGTVCDHRWNLISASVVCRQLGFGSAREALFGARLGQGLGPIHLSEVRCRGYERTLSDCPALEGSQNGCQHENDAAVRCNVPNMGFQNQVRLAGGRIPEEGLLEVQVEVNGVPRWGSVCSENWGLTEAMVACRQLGLGFAIHAYKETWFWSGTPRAQEVVMSGVRCSGTELALQQCQRHGPVHCSHGGGRFLAGVSCMDSAPDLVMNAQLVQETAYLEDRPLSQLYCAHEENCLSKSADHMDWPYGYRRLLRFSTQIYNLGRTDFRPKTGRDSWVWHQCHRHYHSIEVFTHYDLLTLNGSKVAEGHKASFCLEDTNCPTGLQRRYACANFGEQGVTVGCWDTYRHDIDCQWVDITDVGPGNYIFQVIVNPHYEVAESDFSNNMLQCRCKYDGHRVWLHNCHTGNSYPANAELSLEQEQRLRNNLI

SEQ ID NO: 31 - protein, 1722 aa, Homo sapiens
MAGAWLRWGLLLWAGLLASSAHGRLRRITYVVHPGPGLAAGALPLSGPPRSRTFNVALNARYSRSSAAAGAPSRASPGVPSERTRRTSKPGGAALQGLRPPPPPPPEPARPAVPGGQLHPNPGGHPAAAPFTKQGRQVVRSKVPQETQSGGGSRLQVHQKQQLQGVNVCGGRCCHGWSKAPGSQRCTKPSCVPPCQNGGMCLRPQLCVCKPGTKGKACETIAAQDTSSPVFGGQSPGAASSWGPPEQAAKHTSSKKADTLPRVSPVAQMTLTLKPKPSVGLPQQIHSQVTPLSSQSVVIHHGQTQEYVLKPKYFPAQKGISGEQSTEGSFPLRYVQDQVAAPFQLSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPGIVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 32 - protein, 496 aa, Homo sapiens
MRVLSGTSLMLCSLLLLLQALCSPGLAPQSRGHLCRTRPTDLVFVVDSSRSVRPVEFEKVKVFLSQVIESLDVGPNATRVGMVNYASTVKQEFSLRAHVSKAALLQAVRRIQPLSTGTMTGLAIQFAITKAFGDAEGGRSRSPDISKVVIVVTDGRPQDSVQDVSARARASGVELFAIGVGSVDKATLRQIASEPQDEHVDYVESYSVIEKLSRKFQEAFCVVSDLCATGDHDCEQVCISSPGSYTCACHEGFTLNSDGKTCNVCSGGGGSSATDLVFLIDGSKSVRPENFELVKKFISQIVDTLDVSDKLAQVGLVQYSSSVRQEFPLGRFHTKKDIKAAVRNMSYMEKGTMTGAALKYLIDNSFTVSSGARPGAQKVGIVFTDGRSQDYINDAAKKAKDLGFKMFAVGVGNAVEDELREIASEPVAEHYFYTADFKTINQIGKKLQKKICVEEDPCACESLVKFQAKVEGLLQALTRKLEAVSKRLAILENTVV

SEQ ID NO: 33 - protein, 486 aa, Homo sapiens
MPRPAPARRLPGLLLLLWPLLLLPSAAPDPVARPGFRRLETRGPGGSPGRRPSPAAPDGAPASGTSEPGRARGAGVCKSRPLDLVFIIDSSRSVRPLEFTKVKTFVSRIIDTLDIGPADTRVAVVNYASTVKIEFQLQAYTDKQSLKQAVGRITPLSTGTMSGLAIQTAMDEAFTVEAGAREPSSNIPKVAIIVTDGRPQDQVNEVAARAQASGIELYAVGVDRADMASLKMMASEPLEEHVFYVETYGVIEKLSSRFQETFCALDPCVLGTHQCQHVCISDGEGKHHCECSQGYTLNADKKTCSALDRCALNTHGCEHICVNDRSGSYHCECYEGYTLNEDRKTCSAQDKCALGTHGCQHICVNDRTGSHHCECYEGYTLNADKKTCSVRDKCALGSHGCQHICVSDGAASYHCDCYPGYTLNEDKKTCSATEEARRLVSTEDACGCEATLAFQDKVSSYLQRLNTKLDDILEKLKINEYGQIHR

SEQ ID NO: 34 - protein, 525 aa, Homo sapiens
MRVFCVGLLLFSVTWAAPTFQPQTEKTKQSCVEEQRQEEKNKDNIGFHHLGKRINQELSSKENIVQERKKDLSLSEASENKGSSKSQNYFTNRQRLNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 35 - protein, 103 aa, Homo sapiens
MKSLILLAILAALAVVTLCYESHESMESYELNPFINRRNANTFISPQQRWRAKVQERIRERSKPVHELNREACDDYRLCERYAMVYGYNAAYNRYFRKRRGTK

SEQ ID NO: 36 - protein, 2828 aa, Homo sapiens
MPKRAHWGALSVVLILLWGHPRVALACPHPCACYVPSEVHCTFRSLASVPAGIAKHVERINLGFNSIQALSETSFAGLTKLELLMIHGNEIPSIPDGALRDLSSLQVFKFSYNKLRVITGQTLQGLSNLMRLHIDHNKIEFIHPQAFNGLTSLRLLHLEGNLLHQLHPSTFSTFTFLDYFRLSTIRHLYLAENMVRTLPASMLRNMPLLENLYLQGNPWTCDCEMRWFLEWDAKSRGILKCKKDKAYEGGQLCAMCFSPKKLYKHEIHKLKDMTCLKPSIESPLRQNRSRSIEEEQEQEEDGGSQLILEKFQLPQWSISLNMTDEHGNMVNLVCDIKKPMDVYKIHLNQTDPPDIDINATVALDFECPMTRENYEKLWKLIAYYSEVPVKLHRELMLSKDPRVSYQYRQDADEEALYYTGVRAQILAEPEWVMQPSIDIQLNRRQSTAKKVLLSYYTQYSQTISTKDTRQARGRSWVMIEPSGAVQRDQTVLEGGPCQLSCNVKASESPSIFWVLPDGSILKAPMDDPDSKFSILSSGWLRIKSMEPSDSGLYQCIAQVRDEMDRMVYRVLVQSPSTQPAEKDTVTIGKNPGESVTLPCNALAIPEAHLSWILPNRRIINDLANTSHVYMLPNGTLSIPKVQVSDSGYYRCVAVNQQGADHFTVGITVTKKGSGLPSKRGRRPGAKALSRVREDIVEDEGGSGMGDEENTSRRLLHPKDQEVFLKTKDDAINGDKKAKKGRRKLKLWKHSEKEPETNVAEGRRVFESRRRINMANKQINPERWADILAKVRGKNLPKGTEVPPLIKTTSPPSLSLEVTPPFPAISPPSASPVQTVTSAEESSADVPLLGEEEHVLGTISSASMGLEHNHNGVILVEPEVTSTPLEEVVDDLSEKTEEITSTEGDLKGTAAPTLISEPYEPSPTLHTLDTVYEKPTHEETATEGWSAADVGSSPEPTSSEYEPPLDAVSLAESEPMQYFDPDLETKSQPDEDKMKEDTFAHLTPTPTIWVNDSSTSQLFEDSTIGEPGVPGQSHLQGLTDNIHLVKSSLSTQDTLLIKKGMKEMSQTLQGGNMLEGDPTHSRSSESEGQESKSITLPDSTLGIMSSMSPVKKPAETTVGTLLDKDTTTATTTPRQKVAPSSTMSTHPSRRRPNGRRRLRPNKFRHRHKQTPPTTFAPSETFSTQPTQAPDIKISSQVESSLVPTAWVDNTVNTPKQLEMEKNAEPTSKGTPRRKHGKRPNKHRYTPSTVSSRASGSKPSPSPENKHRNIVTPSSETILLPRTVSLKTEGPYDSLDYMTTTRKIYSSYPKVQETLPVTYKPTSDGKEIKDDVATNVDKHKSDILVTGESITNAIPTSRSLVSTMGEFKEESSPVGFPGTPTWNPSRTAQPGRLQTGIPVTTSGENLTDPPLLKELEDVDFTSEFLSSLTVSTPFHQEEAGSSTTLSSIKVEVASSQAETTTLDQDHLETTVAILLSETRPQNHTPTAARMKEPASSSPSTILMSLGQTTTTKPALPSPRISQASRDSKENVFLNYVGNPETEATPVNNEGTQHMSGPNELSTPSSDQDAFNLSTKLELEKQVFGSRSLPRGPDSQRQDGRVHASHQLTRVPAKPILPTATVRLPEMSTQSASRYFVTSQSPRHWTNKPEITTYPSGALPENKQFTTPRLSSTTIPLPLHMSKPSIPSKFTDRRTDQFNGYSKVFGNNNIPEARNPVGKPPSPRIPHYSNGRLPFFTNKTLSFPQLGVTRRPQIPTSPAPVMRERKVIPGSYNRIHSHSTFHLDFGPPAPPLLHTPQTTGSPSTNLQNIPMVSSTQSSISFITSSVQSSGSFHQSSSKFFAGGPPASKFWSLGEKPQILTKSPQTVSVTAETDTVFPCEATGKPKPFVTWTKVSTGALMTPNTRIQRFEVLKNGTLVIRKVQVQDRGQYMCTASNLHGLDRMVVLLSVTVQQPQILASHYQDVTVYLGDTIAMECLAKGTPAPQISWIFPDRRVWQTVSPVEGRITLHENRTLSIKEASFSDRGVYKCVASNAAGADSLAIRLHVAALPPVIHQEKLENISLPPGLSIHIHCTAKAAPLPSVRWVLGDGTQIRPSQFLHGNLFVFPNGTLYIRNLAPKDSGRYECVAANLVGSARRTVQLNVQRAAANARITGTSPRRTDVRYGGTLKLDCSASGDPWPRILWRLPSKRMIDALFSFDSRIKVFANGTLVVKSVTDKDAGDYLCVARNKVGDDYVVLKVDVVMKPAKIEHKEENDHKVFYGGDLKVDCVATGLPNPEISWSLPDGSLVNSFMQSDDSGGRTKRYVVFNNGTLYFNEVGMREEGDYTCFAENQVGKDEMRVRVKVVTAPATIRNKTYLAVQVPYGDVVTVACEAKGEPMPKVTWLSPTNKVIPTSSEKYQIYQDGTLLIQKAQRSDSGNYTCLVRNSAGEDRKTVWIHVNVQPPKINGNPNPITTVREIAAGGSRKLIDCKAEGIPTPRVLWAFPEGVVLPAPYYGNRITVHGNGSLDIRSLRKSDSVQLVCMARNEGGEARLILQLTVLEPMEKPIFHDPISEKITAMAGHTISLNCSAAGTPTPSLVWVLPNGTDLQSGQQLQRFYHKADGMLHISGLSSVDAGAYRCVARNAAGHTERLVSLKVGLKPEANKQYHNLVSIINGETLKLPCTPPGAGQGRFSWTLPNGMHLEGPQTLGRVSLLDNGTLTVREASVFDRGTYVCRMETEYGPSVTSIPVIVIAYPPRITSEPTPVIYTRPGNTVKLNCMAMGIPKADITWELPDKSHLKAGVQARLYGNRFLHPQGSLTIQHATQRDAGFYKCMAKNILGSDSKTTYIHVF

SEQ ID NO: 37 - protein, 442 aa, Homo sapiens
MALPSRILLWKLVLLQSSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 38 - protein, 1375 aa, Homo sapiens
MEGDRVAGRPVLSSLPVLLLLPLLMLRAAALHPDELFPHGESWGDQLLQEGDDESSAVVKLANPLHFYEARFSNLYVGTNGIISTQDFPRETQYVDYDFPTDFPAIAPFLADIDTSHGRGRVLYREDTSPAVLGLAARYVRAGFPRSARFTPTHAFLATWEQVGAYEEVKRGALPSGELNTFQAVLASDGSDSYALFLYPANGLQFLGTRPKESYNVQLQLPARVGFCRGEADDLKSEGPYFSLTSTEQSVKNLYQLSNLGIPGVWAFHIGSTSPLDNVRPAAVGDLSAAHSSVPLGRSFSHATALESDYNEDNLDYYDVNEEEAEYLPGEPEEALNGHSSIDVSFQSKVDTKPLEESSTLDPHTKEGTSLGEVGGPDLKGQVEPWDERETRSPAPPEVDRDSLAPSWETPPPYPENGSIQPYPDGGPVPSEMDVPPAHPEEEIVLRSYPASGHTTPLSRGTYEVGLEDNIGSNTEVFTYNAANKETCEHNHRQCSRHAFCTDYATGFCCHCQSKFYGNGKHCLPEGAPHRVNGKVSGHLHVGHTPVHFTDVDLHAYIVGNDGRAYTAISHIPQPAAQALLPLTPIGGLFGWLFALEKPGSENGFSLAGAAFTHDMEVTFYPGEETVRITQTAEGLDPENYLSIKTNIQGQVPYVSANFTAHISPYKELYHYSDSTVTSTSSRDYSLTFGAINQTWSYRIHQNITYQVCRHAPRHPSFPTTQQLNVDRVFALYNDEERVLRFAVTNQIGPVKEDSDPTPGNPCYDGSHMCDTTARCHPGTGVDYTCECASGYQGDGRNCVDENECATGFHRCGPNSVCINLPGSYRCECRSGYEFADDRHTCILITPPANPCEDGSHTCAPAGQARCVHHGGSTFSCACLPGYAGDGHQCTDVDECSENRCHPAATCYNTPGSFSCRCQPGYYGDGFQCIPDSTSSLTPCEQQQRHAQAQYAYPGARFHIPQCDEQGNFLPLQCHGSTGFCWCVDPDGHEVPGTQTPPGSTPPHCGPSPEPTQRPPTICERWRENLLEHYGGTPRDDQYVPQCDDLGHFIPLQCHGKSDFCWCVDKDGREVQGTRSQPGTTPACIPTVAPPMVRPTPRPDVTPPSVGTFLLYTQGQQIGYLPLNGTRLQKDAAKTLLSLHGSIIVGIDYDCRERMVYWTDVAGRTISRAGLELGAEPETIVNSGLISPEGLAIDHIRRTMYWTDSVLDKIESALLDGSERKVLFYTDLVNPRAIAVDPIRGNLYWTDWNREAPKIETSSLDGENRRILINTDIGLPNGLTFDPFSKLLCWADAGTKKLECTLPDGTGRRVIQNNLKYPFSIVSYADHFYHTDWRRDGVVSVNKHSGQFTDEYLPEQRSHLYGITAVYPYCPTGRK

SEQ ID NO: 39 - protein, 1200 aa, Homo sapiens
MKVAGGLELGAAALLSASPRALVTLSTGPTCSILPKNPLFPQNLSSQPCIKMEGDKSLTFSSYGLQWCLYELDKEEFQTFKELLKKKSSESTTCSIPQFEIENANVECLALLLHEYYGASLAWATSISIFENMNLRTLSEKARDDMKRHSPEDPEATMTDQGPSKEKVPGISQAVQQDSATAAETKEQEISQAMEQEGATAAETEEQEISQAMEQEGATAAETEEQGHGGDTWDYKSHVMTKFAEEEDVRRSFENTAADWPEMQTLAGAFDSDRWGFRPRTVVLHGKSGIGKSALARRIVLCWAQGGLYQGMFSYVFFLPVREMQRKKESSVTEFISREWPDSQAPVTEIMSRPERLLFIIDGFDDLGSVLNNDTKLCKDWAEKQPPFTLIRSLLRKVLLPESFLIVTVRDVGTEKLKSEVVSPRYLLVRGISGEQRIHLLLERGIGEHQKTQGLRAIMNNRELLDQCQVPAVGSLICVALQLQDVVGESVAPFNQTLTGLHAAFVFHQLTPRGVVRRCLNLEERVVLKRFCRMAVEGVWNRKSVFDGDDLMVQGLGESELRALFHMNILLPDSHCEEYYTFFHLSLQDFCAALYYVLEGLEIEPALCPLYVEKTKRSMELKQAGFHIHSLWMKRFLFGLVSEDVRRPLEVLLGCPVPLGVKQKLLHWVSLLGQQPNATTPGDTLDAFHCLFETQDKEFVRLALNSFQEVWLPINQNLDLIASSFCLQHCPYLRKIRVDVKGIFPRDESAEACPVVPLWMRDKTLIEEQWEDFCSMLGTHPHLRQLDLGSSILTERAMKTLCAKLRHPTCKIQTLMFRNAQITPGVQHLWRIVMANRNLRSLNLGGTHLKEEDVRMACEALKHPKCLLESLRLDCCGLTHACYLKISQILTTSPSLKSLSLAGNKVTDQGVMPLSDALRVSQCALQKLILEDCGITATGCQSLASALVSNRSLTHLCLSNNSLGNEGVNLLCRSMRLPHCSLQRLMLNQCHLDTAGCGFLALALMGNSWLTHLSLSMNPVEDNGVKLLCEVMREPSCHLQDLELVKCHLTAACCESLSCVISRSRHLKSLDLTDNALGDGGVAALCEGLKQKNSVLARLGLKACGLTSDCCEALSLALSCNRHLTSLNLVQNNFSPKGMMKLCSAFACPTSNLQIIGLWKWQYPVQIRKLLEEVQLLKPRVVIDGSWHSFDEDDRYWWKN

SEQ ID NO: 40 - protein, 565 aa, Homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNIRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPAGGQYLTVSAAKAPGGKAARLVLPLGRLMHSGDLCLSFRHKVTGLHSGTLQVFVRKHGAHGAALWGRNGGHGWRQTQITLRGADIKSVVFKGEKRRGHTGEIGLDDVSLKKGHCSEER

SEQ ID NO: 41 - protein, 298 aa, Homo sapiens
MKTLQSTLLLLLLVPLIKPAPPTQQDSRIIYDYGTDNFEESIFSQDYEDKYLDGKNIKEKETVIIPNEKSLQLQKDEAITPLPPKKENDEMPTCLLCVCLSGSVYCEEVDIDAVPPLPKESAYLYARFNKIKKLTAKDFADIPNLRRLDFTGNLIEDIEDGTFSKLSLLEELSLAENQLLKLPVLPPKLTLFNAKYNKIKSRGIKANAFKKLNNLTFLYLDHNALESVPLNLPESLRVIHLQFNNIASITDDTFCKANDTSYIRDRIEEIRLEGNPIVLGKHPNSFICLKRLPIGSYF

SEQ ID NO: 42 - protein, 421 aa, Homo sapiens
MGFLSPIYVIFFFFGVKVHCQYETYQWDEDYDQEPDDDYQTGFPFRQNVDYGVPFHQYTLGCVSECFCPTNFPSSMYCDNRKLKTIPNIPMHIQQLYLQFNEIEAVTANSFINATHLKEINLSHNKIKSQKIDYGVFAKLPNLLQLHLEHNNLEEFPFPLPKSLERLLLGYNEISKLQTNAMDGLVNLTMLDLCYNYLHDSLLKDKIFAKMEKLMQLNLCSNRLESMPPGLPSSLMYLSLENNSISSIPEKYFDKLPKLHTLRMSHNKLQDIPYNIFNLPNIVELSVGHNKLKQAFYIPRNLEHLYLQNNEIEKMNLTVMCPSIDPLHYHHLTYIRVDQNKLKEPISSYIFFCFPHIHTIYYGEQRSTNGQTIQLKTQVFRRFPDDDDESEDHDDPDNAHESPEQEGAEGHFDLHYYENQE

SEQ ID NO: 43 - protein, 979 aa, Homo sapiens
MALFAVFQTTFFLTLLSLRTYQSEVLAERLPLTPVSLKVSTNSTRQSLHLQWTVHNLPYHQELKMVFQIQISRIETSNVIWVGNYSTTVKWNQVLHWSWESELPLECATHFVRIKSLVDDAKFPEPNFWSNWSSWEEVSVQDSTGQDILFVFPKDKLVEEGTNVTICYVSRNIQNNVSCYLEGKQIHGEQLDPHVTAFNLNSVPFIRNKGTNIYCEASQGNVSEGMKGIVLFVSKVLEEPKDFSCETEDFKTLHCTWDPGTDTALGWSKQPSQSYTLFESFSGEKKLCTHKNWCNWQITQDSQETYNFTLIAENYLRKRSVNILFNLTHRVYLMNPFSVNFENVNATNAIMTWKVHSIRNNFTYLCQIELHGEGKMMQYNVSIKVNGEYFLSELEPATEYMARVRCADASHFWKWSEWSGQNFTTLEAAPSEAPDVWRIVSLEPGNHTVTLFWKPLSKLHANGKILFYNVVVENLDKPSSSELHSIPAPANSTKLILDRCSYQICVIANNSVGASPASVIVISADPENKEVEEERIAGTEGGFSLSWKPQPGDVIGYVVDWCDHTQDVLGDFQWKNVGPNTTSTVISTDAFRPGVRYDFRIYGLSTKRIACLLEKKTGYSQELAPSDNPHVLVDTLTSHSFTLSWKDYSTESQPGFIQGYHVYLKSKARQCHPRFEKAVLSDGSECCKYKIDNPEEKALIVDNLKPESFYEFFITPFTSAGEGPSATFTKVTTPDEHSSMLIHILLPMVFCVLLIMVMCYLKSQWIKETCYPDIPDPYKSSILSLIKFKENPHLIIMNVSDCIPDAIEVVSKPEGTKIQFLGTRKSLTETELTKPNYLYLLPTEKNHSGPGPCICFENLTYNQAASDSGSCGHVPVSPKAPSMLGLMTSPENVLKALEKNYMNSLGEIPAGETSLNYVSQLASPMFGDKDSLPTNPVEAPHCSEYKMQMAVSLRLALPPPTENSSLSSITLLDPGEHYC

SEQ ID NO: 44 - protein, 133 aa, Homo sapiens
MLDWRLASAHFILAVTLTLWSSGKVLSVDVTTTEAFDSGVIDVQSTPTVREEKSATDLTAKLLLLDELVSLENDVIETKKKRSFSGFGSPLDRLSAGSVDHKGKQRKVVDHPKRRFGIPMDRIGRNRLSNSRG

SEQ ID NO: 45 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 46 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 47 - protein, 168 aa, Homo sapiens
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 48 - protein, 328 aa, Homo sapiens
MMWRWTLMLLLLLLRHWALGKPSPDAGPHGQDRVHHGTPLSEAPHDDAHGNFQYDHEAFLGRDVAKEFDQLTPEESQARLGRIVDRMDLAGDSDGWVSLAELRAWIAHTQQRHIRDSVSAAWHTYDTDRDGRVGWEELRNATYGHYEPGEEFHDVEDAETYKKMLARDERRFRVADQDGDSMATREELTAFLHPEEFPHMRDIVVAETLEDLDKNKDGYVQVEEYIADLYSAEPGEEEPAWVQTERQQFRDFRDLNKDGRLDGSEVGYWVLPPSQDQPLVEANHLLHESDTDKDGRLSKAEILSNWNMFVGSQATNYGEDLTRHHDEL

SEQ ID NO: 49 - protein, 1073 aa, Homo sapiens
MRVFLLCAYILLLMVSQLRAVSFPEDDEPLNTVDYHYSRQYPVFRGRPSGNESQHRLDFQLMLKIRDTLYIAGRDQVYTVNLNEMPKTEVIPNKKLTWRSRQQDRENCAMKGKHKDECHNFIKVFVPRNDEMVFVCGTNAFNPMCRYYRLSTLEYDGEEISGLARCPFDARQTNVALFADGKLYSATVADFLASDAVIYRSMGDGSALRTIKYDSKWIKEPHFLHAIEYGNYVYFFFREIAVEHNNLGKAVYSRVARICKNDMGGSQRVLEKHWTSFLKARLNCSVPGDSFFYFDVLQSITDIIQINGIPTVVGVFTTQLNSIPGSAVCAFSMDDIEKVFKGRFKEQKTPDSVWTAVPEDKVPKPRPGCCAKHGLAEAYKTSIDFPDETLSFIKSHPLMDSAVPPIADEPWFTKTRVRYRLTAISVDHSAGPYQNYTVIFVGSEAGMVLKVLAKTSPFSLNDSVLLEEIEAYNHAKCSAENEEDKKVISLQLDKDHHALYVAFSSCIIRIPLSRCERYGSCKKSCIASRDPYCGWLSQGSCGRVTPGMLAEGYEQDTEFGNTAHLGDCHEILPTSTTPDYKIFGGPTSDMEVSSSSVTTMASIPEITPKVIDTWRPKLTSSRKFVVQDDPNTSDFTDPLSGIPKGVRWEVQSGESNQMVHMNVLITCVFAAFVLGAFIAGVAVYCYRDMFVRKNRKIHKDAESAQSCTDSSGSFAKLNGLFDSPVKEYQQNIDSPKLYSNLLTSRKELPPNGDTKSMVMDHRGQPPELAALPTPESTPVLHQKTLQAMKSHSEKAHGHGASRKETPQFFPSSPPPHSPLSHGHIPSAIVLPNATHDYNTSFSNSNAHKAEKKLQNIDHPLTKSSSKRDHRRSVDSRNTLNDLLKHLNDPNSNPKAIMGDIQMAHQNLMLDPMGSMSEVPPKVPNREASLYSPPSTLPRNSPTKRVDVPTTPGVPMTSLERQRGYHKNSSQRHSISAMPKNLNSPNGVLLSRQPSMNRGGYMPTPTGAKVDYIQGTPVSVHLQPSLSRQSSYTSNGTLPRTGLKRTPSLKPDVPPKPSFVPQTPSVRPLNKYTY

SEQ ID NO: 50 - protein, 398 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITTGSENLHVSHILQKAKIEVSEDGTKASAATTAILIARSSPPWFIVDRPFLFFIRHNPTGAVLFMGQINKP

SEQ ID NO: 51 - protein, 581 aa, Homo sapiens
MPAPRAREQPRVPGERQPLLPRGARGPRRWRRAAGAAVLLVEMLERAAFFGVTANLVLYLNSTNFNWTGEQATRAALVFLGASYLLAPVGGWLADVYLGRYRAVALSLLLYLAASGLLPATAFPDGRSSFCGEMPASPLGPACPSAGCPRSSPSPYCAPVLYAGLLLLGLAASSVRSNLTSFGADQVMDLGRDATRRFFNWFYWSINLGAVLSLLVVAFIQQNISFLLGYSIPVGCVGLAFFIFLFATPVFITKPPMGSQVSSMLKLALQNCCPQLWQRHSARDRQCARVLADERSPQPGASPQEDIANFQVLVKILPVMVTLVPYWMVYFQMQSTYVLQGLHLHIPNIFPANPANISVALRAQGSSYTIPEAWLLLANVVVVLILVPLKDRLIDPLLLRCKLLPSALQKMALGMFFGFTSVIVAGVLEMERLHYIHHNETVSQQIGEVLYNAAPLSIWWQIPQYLLIGISEIFASIPGLEFAYSEAPRSMQGAIMGIFFCLSGVGSLLGSSLVALLSLPGGWLHCPKDFGNINNCRMDLYFFLLAGIQAVTALLFVWIAGRYERASQGPASHSRFSRDRG

SEQ ID NO: 52 - protein, 691 aa, Homo sapiens
MELRSTAAPRAEGYSNVGFQNEENFLENENTSGNNSIRSRAVQSREHTNTKQDEEQVTVEQDSPRNREHMEDDDEEMQQKGCLERRYDTVCGFCRKHKTTLRHIIWGILLAGYLVMVISACVLNFHRALPLFVITVAAIFFVVWDHLMAKYEHRIDEMLSPGRRLLNSHWFWLKWVIWSSLVLAVIFWLAFDTAKLGQQQLVSFGGLIMYIVLLFLFSKYPTRVYWRPVLWGIGLQFLLGLLILRTDPGFIAFDWLGRQVQTFLEYTDAGASFVFGEKYKDHFFAFKVLPIVVFFSTVMSMLYYLGLMQWIIRKVGWIMLVTTGSSPIESVVASGNIFVGQTESPLLVRPYLPYITKSELHAIMTAGFSTIAGSVLGAYISFGVPSSHLLTASVMSAPASLAAAKLFWPETEKPKITLKNAMKMESGDSGNLLEAATQGASSSISLVANIAVNLIAFLALLSFMNSALSWFGNMFDYPQLSFELICSYIFMPFSFMMGVEWQDSFMVARLIGYKTFFNEFVAYEHLSKWIHLRKEGGPKFVNGVQQYISIRSEIIATYALCGFANIGSLGIVIGGLTSMAPSRKRDIASGAVRALIAGTVACFMTACIAGILSSTPVDINCHHVLENAFNSTFPGNTTKVIACCQSLLSSTVAKGPGEVIPGGNHSLYSLKGCCTLLNPSTFNCNGISNTF

SEQ ID NO: 53 - protein, 108 aa, Homo sapiens
MADPRVRQIKIKTGVVKRLVKEKVMYEKEAKQQEEKIEKMRAEDGENYDIKKQAEILQESRMMIPDCQRRLEAAYLDLQRILENEKDLEEAEEYKEARLVLDSVKLEA

SEQ ID NO: 54 - protein, 160 aa, Homo sapiens
MEEGSSSPVSPVDSLGTSEEELERQPKRFGRKRRYSKKSSEDGSPTPGKRGKKGSPSAQSFEELQSQRILANVRERQRTQSLNEAFAALRKIIPTLPSDKLSKIQTLKLAARYIDFLYQVLQSDEMDNKMTSCSYVAHERLSYAFSVWRMEGAWSMSASH

SEQ ID NO: 55 - protein, 140 aa, Homo sapiens
MLYHTVLPTTACSFIVAVNAPFLSFSPLKIREHAGLSCARHSIPHMVDEGGCGSDGSTAVHPGGWTCVPGYQSLRLTSPHVHAALPLAVSTFWVSLKDFTGADFTKAKLFLKEVDMKDLKSGLLKKSKHRLMTYLHPTRF

SEQ ID NO: 56 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSIFMQLPQLNRLTLFGNSLKELSLGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 57 - protein, 12 aa, Homo sapiens
CYAGWLADGSLR

SEQ ID NO: 58 - protein, 10 aa, Homo sapiens
LEGEVFFATR

SEQ ID NO: 59 - protein, 14 aa, Homo sapiens
TPCVGDKDSSPGVR

SEQ ID NO: 60 - protein, 13 aa, Homo sapiens
ACEEAPPSAAHFR

SEQ ID NO: 61 - protein, 13 aa, Homo sapiens
CSAACGQTGVQTR

SEQ ID NO: 62 - protein, 15 aa, Homo sapiens
DRDQPNVSAACLEFK

SEQ ID NO: 63 - protein, 12 aa, Homo sapiens
EIQCSGYTLPTK

SEQ ID NO: 64 - protein, 20 aa, Homo sapiens
FLPSEQIQGVVISVINLEPR

SEQ ID NO: 65 - protein, 15 aa, Homo sapiens
FNPNAIGVPQPYLNK

SEQ ID NO: 66 - protein, 8 aa, Homo sapiens
FYQIEGDR

SEQ ID NO: 67 - protein, 10 aa, Homo sapiens
IVGPLEVNVR

SEQ ID NO: 68 - protein, 10 aa, Homo sapiens
LFNLDVPESR

SEQ ID NO: 69 - protein, 20 aa, Homo sapiens
PRPNSAEESNGPIYAFENLR

SEQ ID NO: 70 - protein, 14 aa, Homo sapiens
SNVGVALTFNCVER

SEQ ID NO: 71 - protein, 9 aa, Homo sapiens
TAFQISMAK

SEQ ID NO: 72 - protein, 10 aa, Homo sapiens
TFLVGNLEIR

SEQ ID NO: 73 - protein, 12 aa, Homo sapiens
ADAGTAVTFQCR

SEQ ID NO: 74 - protein, 34 aa, Homo sapiens
AYANDKFTPSEQVEGVVVTLVNLEPAPGFSANPR

SEQ ID NO: 75 - protein, 13 aa, Homo sapiens
ECQGAPVTASHFR

SEQ ID NO: 76 - protein, 11 aa, Homo sapiens
EMSEAAQAQAR

SEQ ID NO: 77 - protein, 28 aa, Homo sapiens
FTPSEQVEGVVVTLVNLEPAPGFSANPR

SEQ ID NO: 78 - protein, 7 aa, Homo sapiens
GQLYGLR

SEQ ID NO: 79 - protein, 37 aa, Homo sapiens
HPPPVPAEDPAAFSMLAPLDPLGHNYGVYTVTDQSPR

SEQ ID NO: 80 - protein, 13 aa, Homo sapiens
LLESPATALGDIR

SEQ ID NO: 81 - protein, 15 aa, Homo sapiens
PLPATVGVTQPYLDR

SEQ ID NO: 82 - protein, 10 aa, Homo sapiens
RTDHDDPAFK

SEQ ID NO: 83 - protein, 10 aa, Homo sapiens
SHNAGGSHPR

SEQ ID NO: 84 - protein, 9 aa, Homo sapiens
TDHDDPAFK

SEQ ID NO: 85 - protein, 8 aa, Homo sapiens
VAVNGLLR

SEQ ID NO: 86 - protein, 14 aa, Homo sapiens
VEADKYEYNVVPFR

SEQ ID NO: 87 - protein, 10 aa, Homo sapiens
VFLVGNVEIR

SEQ ID NO: 88 - protein, 17 aa, Homo sapiens
ITQTAEGLDPENYLSIK

SEQ ID NO: 89 - protein, 14 aa, Homo sapiens
AGDGDGWVSLAELR

SEQ ID NO: 90 - protein, 8 aa, Homo sapiens
CYYFSVEK

SEQ ID NO: 91 - protein, 15 aa, Homo sapiens
CYYFSVEKEIFEDAK

SEQ ID NO: 92 - protein, 10 aa, Homo sapiens
DRETVLSSAL

SEQ ID NO: 93 - protein, 7 aa, Homo sapiens
EEQQWIK

SEQ ID NO: 94 - protein, 8 aa, Homo sapiens
EEQQWIKK

SEQ ID NO: 95 - protein, 7 aa, Homo sapiens
EIFEDAK

SEQ ID NO: 96 - protein, 12 aa, Homo sapiens
ESHWIGLTDSER

SEQ ID NO: 97 - protein, 17 aa, Homo sapiens
ESHWIGLTDSERENEWK

SEQ ID NO: 98 - protein, 10 aa, Homo sapiens
SSHLVFINTR

SEQ ID NO: 99 - protein, 10 aa, Homo sapiens
WLDGTSPDYK

SEQ ID NO: 100 - protein, 8 aa, Homo sapiens
AALAQLLK

SEQ ID NO: 101 - protein, 12 aa, Homo sapiens
GIAEGTFSHLTK

SEQ ID NO: 102 - protein, 22 aa, Homo sapiens
LEELHLDDNSISTVGVEDGAFR

SEQ ID NO: 103 - protein, 11 aa, Homo sapiens
LIVDGNLLTNK

SEQ ID NO: 104 - protein, 20 aa, Homo sapiens
LYLQDNQINHIPLTAFSNLR

SEQ ID NO: 105 - protein, 14 aa, Homo sapiens
MLTQGVFDNLSNLK

SEQ ID NO: 106 - protein, 18 aa, Homo sapiens
NHLSSVPVGLPVDLQELR

SEQ ID NO: 107 - protein, 16 aa, Homo sapiens
ISTISSPSLQGLTSLK

SEQ ID NO: 108 - protein, 17 aa, Homo sapiens
IYLYHNSLDEFPTNLPK

SEQ ID NO: 109 - protein, 8 aa, Homo sapiens
AWGPHCEK

SEQ ID NO: 110 - protein, 7 aa, Homo sapiens
FSEQQRK

SEQ ID NO: 111 - protein, 15 aa, Homo sapiens
MTCVDVNECDELNNR

SEQ ID NO: 112 - protein, 12 aa, Homo sapiens
QCMHPLSVHLTK

SEQ ID NO: 113 - protein, 9 aa, Homo sapiens
QLCCCSVGK

SEQ ID NO: 114 - protein, 19 aa, Homo sapiens
VQEGYTCDCFDGYHLDTAK

SEQ ID NO: 115 - protein, 13 aa, Homo sapiens
VMIEPSGPIHVPK

SEQ ID NO: 116 - protein, 9 aa, Homo sapiens
HGECIGPNK

SEQ ID NO: 117 - protein, 7 aa, Homo sapiens
LVLPLGR

SEQ ID NO: 118 - protein, 26 aa, Homo sapiens
EKDNDLHWEPIRDPAGGQYLTVSAAK

SEQ ID NO: 119 - protein, 12 aa, Homo sapiens
KHGAHGAALWGR

SEQ ID NO: 120 - protein, 14 aa, Homo sapiens
DPAGGQYLTVSAAK

SEQ ID NO: 121 - protein, 11 aa, Homo sapiens
HGAHGAALWGR

SEQ ID NO: 122 - protein, 14 aa, Homo sapiens
VTGLHSGTLQVFVR

SEQ ID NO: 123 - protein, 12 aa, Homo sapiens
TCVDVDECATGR

SEQ ID NO: 124 - protein, 7 aa, Homo sapiens
QTQITLR

SEQ ID NO: 125 - protein, 12 aa, Homo sapiens
QCVNTFGSYICK

SEQ ID NO: 126 - protein, 15 aa, Homo sapiens
EGYQGDGLTCVYIPK

SEQ ID NO: 127 - protein, 10 aa, Homo sapiens
EESEADQWLR

SEQ ID NO: 128 - protein, 18 aa, Homo sapiens
LLLLDELVSLENDVIETK

SEQ ID NO: 129 - protein, 8 aa, Homo sapiens
SATDLTAK

SEQ ID NO: 130 - protein, 9 aa, Homo sapiens
DFADIPNLR

SEQ ID NO: 131 - protein, 18 aa, Homo sapiens
LDFTGNLIEDIEDGTFSK

SEQ ID NO: 132 - protein, 16 aa, Homo sapiens
LSLLEELSLAENQLLK

SEQ ID NO: 133 - protein, 19 aa, Homo sapiens
RLDFTGNLIEDIEDGTFSK

SEQ ID NO: 134 - protein, 12 aa, Homo sapiens
LLGEENKENTPR

SEQ ID NO: 135 - protein, 8 aa, Homo sapiens
AYGPLFLR

SEQ ID NO: 136 - protein, 12 aa, Homo sapiens
DRVAVGADAFER

SEQ ID NO: 137 - protein, 7 aa, Homo sapiens
EVLAVAR

SEQ ID NO: 138 - protein, 11 aa, Homo sapiens
GAPALLTCVNR

SEQ ID NO: 139 - protein, 7 aa, Homo sapiens
GHVWTDR

SEQ ID NO: 140 - protein, 8 aa, Homo sapiens
GPGGGPAR

SEQ ID NO: 141 - protein, 13 aa, Homo sapiens
HVEEAQQVVHWDR

SEQ ID NO: 142 - protein, 18 aa, Homo sapiens
LEVTDGPPATPAYWDGEK

SEQ ID NO: 143 - protein, 25 aa, Homo sapiens
LEVTDGPPATPAYWDGEKEVLAVAR

SEQ ID NO: 144 - protein, 10 aa, Homo sapiens
LLDLYASGER

SEQ ID NO: 145 - protein, 11 aa, Homo sapiens
LLDLYASGERR

SEQ ID NO: 146 - protein, 11 aa, Homo sapiens
LLDLYSAGEQR

SEQ ID NO: 147 - protein, 7 aa, Homo sapiens
MVWTQDR

SEQ ID NO: 148 - protein, 9 aa, Homo sapiens
QPPGVPHDR

SEQ ID NO: 149 - protein, 9 aa, Homo sapiens
RAYGPLFLR

SEQ ID NO: 150 - protein, 12 aa, Homo sapiens
RLLDLYSAGEQR

SEQ ID NO: 151 - protein, 17 aa, Homo sapiens
RVFHLTVAEPHAEPPPR

SEQ ID NO: 152 - protein, 10 aa, Homo sapiens
VAVGADAFER

SEQ ID NO: 153 - protein, 16 aa, Homo sapiens
VFHLTVAEPHAEPPPR

SEQ ID NO: 154 - protein, 7 aa, Homo sapiens
VLHWDLR

SEQ ID NO: 155 - protein, 12 aa, Homo sapiens
DIVTVANAVFVK

SEQ ID NO: 156 - protein, 10 aa, Homo sapiens
LEAAYLDLQR

SEQ ID NO: 157 - protein, 10 aa, Homo sapiens
LVLDSVKLEA

SEQ ID NO: 158 - protein, 11 aa, Homo sapiens
RLEAAYLDLQR

SEQ ID NO: 159 - protein, 21 aa, Homo sapiens
EALGDVVGDYNFICPALEFTK

SEQ ID NO: 160 - protein, 11 aa, Homo sapiens
IFFPGVSEFGK

SEQ ID NO: 161 - protein, 10 aa, Homo sapiens
DKQHFTTLIK

SEQ ID NO: 162 - protein, 15 aa, Homo sapiens
EAGTLAYYEICDFLR

SEQ ID NO: 163 - protein, 11 aa, Homo sapiens
ILGQQVPYATK

SEQ ID NO: 164 - protein, 12 aa, Homo sapiens
QLLLSAALSAGK

SEQ ID NO: 165 - protein, 23 aa, Homo sapiens
SFTLASSETGVGAPISGPGIPGR

SEQ ID NO: 166 - protein, 16 aa, Homo sapiens
THGFDGLDLAWLYPGR

SEQ ID NO: 167 - protein, 14 aa, Homo sapiens
TLLSVGGWNFGSQR

SEQ ID NO: 168 - protein, 11 aa, Homo sapiens
VTIDSSYDIAK

SEQ ID NO: 169 - protein, 14 aa, Homo sapiens
QDADEEALYYTGVR

SEQ ID NO: 170 - protein, 13 aa, Homo sapiens
YQIYQDGTLLIQK

SEQ ID NO: 171 - protein, 10 aa, Homo sapiens
HLYLAENMVR

SEQ ID NO: 172 - protein, 9 aa, Homo sapiens
VDVVMKPAK

SEQ ID NO: 173 - protein, 9 aa, Homo sapiens
VGLKPEANK

SEQ ID NO: 174 - protein, 10 aa, Homo sapiens
VQVSDSGYYR

SEQ ID NO: 175 - protein, 14 aa, Homo sapiens
HSEKEPETNVAEGR

SEQ ID NO: 176 - protein, 9 aa, Homo sapiens
RTVQLNVQR

SEQ ID NO: 177 - protein, 10 aa, Homo sapiens
ADITWELPDK

SEQ ID NO: 178 - protein, 8 aa, Homo sapiens
TVQLNVQR

SEQ ID NO: 179 - protein, 11 aa, Homo sapiens
LIAYYSEVPVK

SEQ ID NO: 180 - protein, 7 aa, Homo sapiens
TTYIHVF

SEQ ID NO: 181 - protein, 7 aa, Homo sapiens
CFESFER

SEQ ID NO: 182 - protein, 11 aa, Homo sapiens
GRECDCDAQCK

SEQ ID NO: 183 - protein, 9 aa, Homo sapiens
VCTAELSCK

SEQ ID NO: 184 - protein, 24 aa, Homo sapiens
AYYNGISLFNNPVPYWEVQPATFR

SEQ ID NO: 185 - protein, 20 aa, Homo sapiens
EISPDTTLLDLQNNDISELR

SEQ ID NO: 186 - protein, 21 aa, Homo sapiens
EISPDTTLLDLQNNDISELRK

SEQ ID NO: 187 - protein, 11 aa, Homo sapiens
IQAIELEDLLR

SEQ ID NO: 188 - protein, 9 aa, Homo sapiens
LGLGHNQIR

SEQ ID NO: 189 - protein, 18 aa, Homo sapiens
NHLVEIPPNLPSSLVELR

SEQ ID NO: 190 - protein, 14 aa, Homo sapiens
VGVNDFCPMGFGVK

SEQ ID NO: 191 - protein, 10 aa, Homo sapiens
VVQCSDLGLK

SEQ ID NO: 192 - protein, 10 aa, Homo sapiens
LLLGYNEISK

SEQ ID NO: 193 - protein, 23 aa, Homo sapiens
LQDIPYNIFNLPNIVELSVGHNK

SEQ ID NO: 194 - protein, 20 aa, Artificial Sequence
  synthetic peptide
RLIDGSSPQEPEFTGVLGPH

SEQ ID NO: 195 - protein, 15 aa, Artificial Sequence
  Synthetic peptide
INKNDFASLSDLKRI

SEQ ID NO: 196 - protein, 18 aa, Artificial Sequence
  Synthetic peptide
FIDISNNRLGRKGIKQEA

SEQ ID NO: 197 - protein, 11 aa, Artificial Sequence
  Synthetic peptide
ENKVKKIQKDT

SEQ ID NO: 198 - protein, 10 aa, Artificial Sequence
  Synthetic peptide
LKKIPSGLPE

SEQ ID NO: 199 - protein, 11 aa, Artificial Sequence
  Synthetic peptide
KKSLYSAISLF

SEQ ID NO: 200 - protein, 11 aa, Artificial Sequence
  Synthetic peptide
ISSSASSSLET

SEQ ID NO: 201 - protein, 10 aa, Artificial Sequence
  Synthetic peptide
NDIESKSLVL

SEQ ID NO: 202 - protein, 16 aa, Artificial Sequence
  Synthetic peptide
PVYAASSGQKKQQQSK

SEQ ID NO: 203 - protein, 16 aa, Artificial Sequence
  Synthetic peptide
AAEENCLASSARSANW

SEQ ID NO: 204 - protein, 15 aa, Artificial Sequence
  Synthetic peptide
ILTPNGTKVAEGHKA

SEQ ID NO: 205 - protein, 30 aa, Artificial Sequence
  Synthetic peptide
ELERQPKRFGRKRRYSKKSSEDGSPTPGKR

SEQ ID NO: 206 - protein, 20 aa, Artificial Sequence
  Synthetic peptide
KIIPTLPSDKLSKIQTLKLA

SEQ ID NO: 207 - protein, 20 aa, Artificial Sequence
  Synthetic peptide
TGGRGVSVGPILSSSASDIF

SEQ ID NO: 208 - protein, 20 aa, Artificial Sequence
  Synthetic peptide
VNTYGSYRCRTNKKCSRGYE

SEQ ID NO: 209 - protein, 20 aa, Artificial Sequence
  Synthetic peptide
AQKRLVNIAVDERSSPYYAL

SEQ ID NO: 210 - protein, 20 aa, Artificial Sequence
  Synthetic peptide
DRNQLSSYPSAALSKLRVVE

SEQ ID NO: 211 - protein, 17 aa, Artificial Sequence
  Synthetic peptide
FRSCKFPTKRSKKAGRH

(19) United States
(12) Patent Application Publication
(10) Pub. No.: US 2011/0189694 A1
(43) Pub. Date: Aug. 4, 2011

(54) METHODS FOR DETECTION AND DIAGNOSIS OF A BONE OR CARTILAGE DISORDER

(75) Inventors: Wolfgang Woloszczuk, Vienna (AT); Gerhard Hawa, Vienna (AT)

(73) Assignee: BIOMARKER DESIGN FORSCHUNGS GMBH, Vienna (AT)

(21) Appl. No.: 13/122,541

(22) PCT Filed: Oct. 22, 2009
(86) PCT No.: PCT/EP2009/063909

§ 371 Date: Apr. 20, 2011

Related U.S. Application Data
(60) Provisional application No. 61/107,564, filed on Oct. 22, 2008.

(51) Int. Cl. G01N 33/53; G01N 33/566; C07K 16/00

(52) U.S. Cl. 435/7.1; 436/501; 530/387.9

(57) ABSTRACT

The present invention is directed to methods for the detection and diagnosis of bone and/or cartilage disorders, wherein the level of expression of a polypeptide in a test sample is measured by contacting the test sample with an antibody that specifically binds to said polypeptide and measuring the binding of said antibody to said test sample.

### FIELD OF THE INVENTION

The present invention relates to methods and kits for diagnosing and/or detecting a bone or cartilage disorder. More specifically, the present invention identifies several genes expressed at higher level in bone and/or cartilage which may be useful in the methods. The present invention also identifies several peptides occurring in body fluids, which may be useful in the methods.

### BACKGROUND

Bones are rigid, dynamic organs comprising a variety of tissue types. The primary tissue of bone is osseous tissue, also called bone tissue, a relatively hard, lightweight tissue formed mostly of calcium phosphate. Other tissue types found in bone include marrow, endosteum and periosteum, nervers, blood vessels and cartilage.

Two distinct cell lineages are found in bone. Osteoblasts are bone-forming cells which descend from undifferentiated mesenchymal progenitor cells. Osteoclasts are the cells responsible for bone resorption and descend from monocyte stem cells. The continuous destruction and rebuilding of bone arises from the interplay of these two cell types. The action of osteoblasts and osteoclasts are controlled by chemical factors which either promote or inhibit the activity of these cells thus controlling the rate at which bone is made and/or destroyed.

The formation of bone during embryonic development occurs through two distinct pathways: endochondrial or intramembranous ossification. Intramembranous ossification occurs mainly during formation of bones of the skull from mesenchyme tissue. Endochondral ossification occurs in long bones (e.g. limbs) and entails the formation of bone from cartilage. Endochondrial ossification begins at primary ossification centers in the cartilage which appear during fetal development. These centers are responsible for the formation of the diaphyses of long bones. Secondary ossification occurs after birth, and forms the epiphyses of long bones. The diaphysis and both epiphyses of a long bone are separated by a growing zone of cartilage called the epiphyseal plate. When maturity is reached (about 18 years of age for humans), the cartilage is replaced by bone, fusing the diaphysis and both epiphyses together, a process termed epiphyseal closure.

A panoply of disorders arise from disturbances in the complex balance of bone resporption and build up, including but not limited to Paget's disease (osteitis deformans), inflammatory bone diseases such as rheumatoid arthritis, osteoarthritis and periodontal disease, focal osteogenesis occurring during skeletal metastases, Crouzon's syndrome, opsismodysplasia, pycnodysostosis/Toulouse-Lautrec disease, osteogenesis imperfecta (brittle bone disease) and osteoporosis. Other skeletal disorders include osteomalacia, osteopenia, osteopetrosis and osteochondritis dissecans.

Osteoporosis is one of the most prominent of the skeletal disorders. In osteoporosis, the bone mineral density is reduced, bone architecture is disrupted and the amount and variety of non-collagenous proteins in the bone is altered. The disease is most commonly observed in post-menopausal females but may also develop in men. The underlying mechanism in osteoporosis is an imbalance between bone resorption and bone formation; bone resorption is increased and/or formation of new bone is inadequate.

Cartilage is a type of dense connective tissue composed of specialized cells called chondrocytes that produce extracellular matrix. Cartilage is found in the articular surface of bones, the rib cage, the ear, the nose, the bronchial tubes and the intervertebral discs. Its mechanical properties lie between bone and dense connective tissue. Unlike other connective tissue, cartilage does not contain blood vessels.

Cartilage is classified into three groups: hyaline cartilage, elastic cartilage and fibrocartilage. Hyaline cartilage is a hard, translucent material with high concentrations of collagen and proteoglycan. It covers the ends of bones to form the smooth articular surface of joints. Elastic cartilage contains high concentrations of elastin, which provides an elasticity, and is found in the pinna of the ear, in tubular structures such as the auditory tubes and in the epioglotis. Fibrocartilage comprises a dense network of type I collagen providing high tensile strength and support, and is found in the intervertebral discs, the symphysis pubis and the attachments of certain tendons and ligaments.

Several diseases affect the cartilage including osteoarthritis, achondroplasia, and costochondritis. Osteoarthritis is a clinical syndrome in which low-grade inflammation results in pain in the joints, caused by abnormal wearing of the cartilage inside joints or a decrease of synovial fluid that lubricates the joints. The main symptom of osteoarthritis is chronic pain resulting in decreased mobility. There appears to be a hereditary susceptibility to this condition.

In recent years the palette of biomarkers to study the biological processes leading to severe bone and cartilage diseases like osteoporosis and rheumatoid arthritis has increased. Recently, the focus was drawn to several regulation molecules influencing either osteoclast or osteoblast proliferation. Despite this progress in understanding the biology of bone turnover, more biomarkers are needed for basic research and clinical studies on bone and cartilage disease.

### SUMMARY

The present invention relates to methods of diagnosis and detection in the field of bone and/or cartilage disorders based on the differential expression observed for the polypeptides of the invention represented by SEQ ID NOs: 1 to 56. The present invention also relates to methods of diagnosis and detection in the field of bone and/or cartilage disorders based on the detection of body fluid peptides, represented by SEQ ID NOs: 57-193, derived from the polypeptides of the invention.

The present specification describes the identification of various polypeptides which are expressed to a greater degree in bone and/or cartilage tissue as compared to other tissues. These polypeptides are expected to serve as effective targets for the diagnosis and detection of bone and/or cartilage disorders in mammals. It appears that the polypeptides are differentially expressed in patients exhibiting a bone and/or cartilage disorder relative to healthy age and gender matched controls. The skilled artisan will recognize that such differentially expressed polypeptides have utility in the early detection, diagnosis, and/or prognosis of bone disorders, within the scope of the present invention.

The present specification also describes the identification of body fluid peptides derived from the selectively expressed polypeptides. The body fluid peptides are also found to serve as effective targets for the diagnosis and detection of bone and/or cartilage disorders in mammals. Generally, the detection for use in the diagnosis is performed in an in vitro assay.

Bone disorders which may be diagnosed according to the present invention include, without limitation, osteoporosis, osteopenia, osteomalacia, osteomyeloma, osteodystrophy, Paget's disease, osteogenesis imperfecta, bone sclerosis, aplastic bone disorder, humoral hypercalcemic myeloma, multiple myeloma, Crouzon's syndrome, opsismodysplasia, pycnodysostosis, and osteopetrosis.

Cartilage disorders which may be diagnosed according to the present invention include, without limitation, arthritis, including osteoarthritis and rheumatoid arthritis, degenerative joint disease, osteochondritis, osteochondritis dissecans, costochondritis and polychondritis.

In one embodiment, the present invention provides an antibody which binds, preferably specifically, to a polypeptide selected from SEQ ID NOs: 1 to 56. In a related embodiment, the present invention provides an antibody which binds, preferably specifically, to a peptide selected from SEQ ID NOs: 57-193. Optionally, the antibody is a monoclonal antibody, antibody fragment, chimeric antibody, humanized antibody or single-chain antibody. For diagnostic purposes, the antibodies of the present invention may be detectably labeled, attached to a solid support, or the like. The antibody may bind to any epitope of a polypeptide selected from SEQ ID NOs: 1 to 56 and is capable of binding to at least one epitope of the polypeptide. The antibody may recognize a linear or conformational epitope of a polypeptide selected from SEQ ID NOs: 1 to 56. In one embodiment, the antibody recognizes an epitope common to a polypeptide selected from SEQ ID NOs: 1 to 56 and a peptide selected from SEQ ID NOs: 57-193.

In other embodiments, the present invention provides DNA encoding any of the herein described antibodies and vectors comprising the DNA. Host cells comprising any such vector are also provided. By way of example, the host cells may be CHO cells, E. coli cells or yeast cells.

In other embodiments, the present invention provides oligopeptides which bind, preferably specifically, to a polypeptide selected from SEQ ID NOs: 1 to 56. In a related embodiment, the present invention provides oligopeptides which bind, preferably specifically, to a peptide selected from SEQ ID NOs: 57-193. The oligopeptides of the present invention may optionally be produced in CHO cells or bacterial cells. For diagnostic purposes, the oligopeptides may be detectably labeled, attached to a solid support or the like. Vectors comprising DNA encoding any oligopeptide of the invention and host cells comprising any such vector are also provided. A process for producing any of the oligopeptides is further provided and comprises culturing host cells under conditions suitable for expression of the desired oligopeptide and recovering the desired oligopeptide from the cell culture.

In another embodiment, the invention provides small organic molecules which bind, preferably specifically, to a polypeptide selected from SEQ ID NOs: 1 to 56 and/or a peptide selected from SEQ ID NOs: 57-193. For diagnostic purposes, the organic molecules of the present invention may be detectably labeled, attached to a solid support, or the like.

In another embodiment, the invention concerns a composition comprising any of the herein described antibodies, oligopeptides or small organic molecules (collectively, “specific binding agents”), in combination with a carrier. Optionally, the carrier is a pharmaceutically acceptable carrier.

In yet another embodiment, the invention concerns a kit comprising a container and a composition within the container, wherein the composition may comprise any of the herein described specific binding agents (i.e. antibodies, oligopeptides or small organic molecules). The kit may further optionally comprise a label affixed to the container, or a package insert included with the container, that refers to the use of the composition for the diagnostic detection of a bone and/or cartilage disorder.

Yet another embodiment of the present invention is directed to a method of determining the level of expression of a polypeptide selected from SEQ ID NOs: 1 to 56 and/or a peptide selected from SEQ ID NOs: 57-193 in a test sample. In one aspect, the method comprises contacting the test sample with a specific binding agent (i.e. an antibody, oligopeptide or small organic molecule) that specifically binds the polypeptide and/or peptide and determining the binding of the specific binding agent to the test sample. Preferably, the specific binding agent is an antibody.

Yet another embodiment of the present invention is directed to a method of determining altered expression of a polypeptide selected from SEQ ID NOs: 1 to 56 in a test sample comprising (a) contacting a test sample with a specific binding agent that specifically binds to said polypeptide (b) measuring binding of said specific binding agent to said test sample and (c) comparing binding of step (b) to a control, whereby altered expression of said polypeptide is identified by a difference in binding of step (b) to a control. Preferably, the specific binding agent is an antibody.

In a related embodiment, the present invention provides a method of determining altered expression of a polypeptide selected from SEQ ID NOs: 1 to 56 in a test sample comprising (a) contacting a test sample with a specific binding agent that specifically binds to a peptide of SEQ ID NOs: 57-193 (b) measuring binding of said specific binding agent to said test sample and (c) comparing binding of step (b) to a control, whereby altered expression of said polypeptide is identified by a difference in binding of step (b) to a control. Preferably, the specific binding agent is an antibody.

Yet another embodiment of the present invention is directed to a method of determining altered expression of a peptide selected from SEQ ID NOs: 57 to 193 in a test sample comprising (a) contacting a test sample with a specific binding agent that specifically binds to said peptide (b) measuring binding of said specific binding agent to said test sample and (c) comparing binding of step (b) to a control, whereby altered expression of said peptide is identified by a difference in binding of step (b) to a control. Preferably, the specific binding agent is an antibody.

In a related embodiment, the present invention provides a method of determining altered expression of a peptide selected from SEQ ID NOs: 57 to 193 in a test sample comprising (a) contacting a test sample with a specific binding agent that specifically binds to a polypeptide of SEQ ID NOs: 1-56 (b) measuring binding of said specific binding agent to said test sample and (c) comparing binding of step (b) to a control, whereby altered expression of said peptide is identified by a difference in binding of step (b) to a control. Preferably, the specific binding agent is an antibody.

Yet another embodiment of the present invention is directed to a method of diagnosing a bone and/or cartilage disorder in a mammal, comprising determining the level of a polypeptide selected from SEQ ID NOs: 1 to 56 in a test sample from said mammal suspected of having said disorder, wherein a difference in the level of said polypeptide in said mammal compared to the level of said polypeptide in a normal mammal indicates the presence of a bone and/or cartilage disorder. Preferably the mammal is a human.

In a related embodiment, the present invention provides a method of diagnosing a bone and/or cartilage disorder in a mammal, comprising determining the level of a peptide selected from SEQ ID NOs: 57-193 in a test sample from said mammal suspected of having said disorder, wherein a difference in the level of said peptide in said mammal compared to the level of said peptide in a normal mammal indicates the presence of a bond and/or cartilage disorder. Preferably the mammal is a human.

In one aspect, the diagnostic method comprises (a) contacting a test sample obtained from the mammal with a specific binding agent that binds to a polypeptide selected from SEQ ID NOs: 1 to 56 (b) measuring binding of the specific binding agent to the test sample and (c) comparing binding of step (b) to a control, whereby a bone and/or cartilage disorder is diagnosed by an increase or decrease in expression of the polypeptide in the test sample compared to control. Preferably, the specific binding agent is an antibody.

In a related aspect, the diagnostic method comprises (a) contacting a test sample obtained from the mammal with a specific binding agent that binds to a peptide selected from SEQ ID NOs: 57-193 (b) measuring binding of the specific binding agent to the test sample and (c) comparing binding of step (b) to a control, whereby a bone and/or cartilage disorder is diagnosed by an increase or decrease in binding to the test sample compared to control. Preferably, the specific binding agent is an antibody.

Yet another embodiment of the present invention is directed to a method of binding a specific binding agent to a test sample comprising a polypeptide selected from SEQ ID NOs: 1 to 56 and/or a peptide selected from SEQ ID NOs: 57-193, wherein the method comprises contacting the test sample with said specific binding agent under conditions which are suitable for binding of the specific binding agent to said polypeptide and/or peptide and allowing binding therebetween. Preferably, the specific binding agent is an antibody.

Other embodiments of the present invention are directed to the use of a herein described specific binding agent in the preparation of a medicament useful for the diagnostic detection of a bone and/or cartilage disorder. Preferably, the specific binding agent is an antibody.

### BRIEF DESCRIPTION OF THE DRAWINGS

FIGS. 1A-1C illustrate recognition factor, accessibility and polarity profiles for epiphycan (EPYC) (SEQ ID NO: 19).

FIGS. 2A-2C illustrate recognition factor, accessibility and polarity profiles for asporin (ASPN) (SEQ ID NO: 2).

FIGS. 3A-3C illustrate recognition factor, accessibility and polarity profiles for LOC 646627 (SEQ ID NO: 28).

FIGS. 4A-4C illustrate recognition factor, accessibility and polarity profiles for LOXL3 (SEQ ID NO: 29).

FIGS. 5A-5C illustrate recognition factor, accessibility and polarity profiles for TWIST2 (SEQ ID NO: 54).

FIGS. 6A-6C illustrate recognition factor, accessibility and polarity profiles for CRTAC1 (SEQ ID NO: 16).

FIGS. 7A-7C illustrate recognition factor, accessibility and polarity profiles for CHAD (SEQ ID NO: 10).

### DETAILED DESCRIPTION

Definitions

By “test sample” is intended any biological sample obtained from an organism, body fluids, cell line, tissue culture, or other source which contains, or is suspected to contain, a polypeptide selected from SEQ ID NOs: 1 to 56 and/or a peptide selected from SEQ ID NOs: 57-193. As indicated, biological samples include body fluids (such as the following non-limiting examples, sputum, amniotic fluid, urine, saliva, tears, sweat, breast milk, secretions, interstitial fluid, blood, synovial fluid, serum, spinal fluid, lymph, semen, vaginal fluid, cerebro-spinal fluid, cell culture supernatant, cell extract, tissue extract, etc.) which contain the polypeptides and/or peptides, and other tissue sources found to express the polypeptides and/or peptides. Methods for obtaining tissue biopsies and body fluids from organisms are well known in the art.

The term “antibody” is used in the broadest sense and specifically covers, for example, single monoclonal antibodies (including agonist, antagonist and neutralizing antibodies), antibody compositions with polyepitopic specificity, polyclonal antibodies, single chain antibodies, and antibody fragments that exhibit the desired biological or immunological activity. The term “immunoglobulin” (Ig) is used interchangeable with antibody herein.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contains 10 antigen binding sites, while secreted IgA antibodies can polymerize to form polyvalent assemblages comprising 2-5 of the basic 4-chain units along with J chain). In the case of IgGs, the 4-chain unit is generally about 150 kDa. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobluins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Rather, the V regions consist of relatively invariant stretches called framework regions (FR) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable or complementarity determining region (CDR), which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity.

The term “monoclonal antibody” as used herein refers to an antibody obtained from a population of substantially homogenous antibodies, i.e., the individual antibodies comprising the population are identical except for possibly naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology or may be made using recombinant DNA methods in bacterial, eukaryotic animals or plant cells or may be isolated from phage antibody libraries.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C_{L} and at least heavy chain constant domains C_{H}1, C_{H}2 and C_{H}3.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding.

The term “diabodies” refers to small antibody fragments prepared by constructing sFv fragments with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

An antibody or other organic molecule “which binds” an antigen of interest, e.g. a polypeptide selected from SEQ ID NOs: 1 to 56 and/or a peptide selected from SEQ ID NOs: 57-193, is one that binds the antigen with sufficient affinity such that the antibody or other organic molecule is useful as a diagnostic agent in a cell, tissue and/or body fluid expressing the antigen, and does not significantly cross-react with other proteins. In such embodiments, the extent of binding of the antibody or other organic molecule to a “non-target” protein will be less than about 10% of the binding of the antibody or other organic molecule to its particular target protein as determined by fluorescence activated cell sorting (FACS) analysis or radioimmunoprecipitation (RIA). With regard to the binding of an antibody or other organic molecule to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope

The word “label” when used herein refers to a detectable compound or composition which is conjugated directly or indirectly to the antibody, oligopeptide or other organic molecule so as to generate a “labeled” antibody, oligopeptide or other organic molecule. The label may be detectable by itself (e.g. radioisotope labels or fluorescent labels) or, in the case of an enzymatic label, may catalyze chemical alteration of a substrate compound or composition which is detectable.

The terms “Western blot,” “Western immunoblot” “immunoblot” and “Western” refer to the immunological analysis of protein(s), polypeptides or peptides that have been immobilized onto a membrane support. The proteins are first resolved by polyacrylamide gel electrophoresis (i.e., SDS-PAGE) to separate the proteins, followed by transfer of the protein from the gel to a solid support, such as nitrocellulose or a nylon membrane. The immobilized proteins are then exposed to an antibody having reactivity towards an antigen of interest. The binding of the antibody (i.e., the primary antibody) is detected by use of a secondary antibody which specifically binds the primary antibody. The secondary antibody is typically conjugated to an enzyme which permits visualization of the antigen-antibody complex by the production of a colored reaction product or catalyzes a luminescent enzymatic reaction (e.g., the ECL reagent, Amersham).

As used herein, the term “ELISA” refers to enzyme-linked immunosorbent assay (or EIA). Numerous ELISA methods and applications are known in the art, and are described in many references (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). In addition, there are numerous commercially available ELISA test systems.

One ELISA method is a “direct ELISA,” where an antigen (e.g., a polypeptide selected from SEQ ID Nos: 1-56 and/or a peptide selected from SEQ ID Nos: 57-193) in a sample is detected. In one embodiment of the direct ELISA, a sample containing antigen is exposed to a solid (i.e., stationary or immobilized) support (e.g., a microtiter plate well). The antigen within the sample becomes immobilized to the stationary phase, and is detected directly using an enzyme-conjugated antibody specific for the antigen.

In an alternative embodiment, an antibody specific for an antigen is detected in a sample. In this embodiment, a sample containing an antibody (e.g., an antibody specific for a polypeptide selected from SEQ ID Nos: 1-56 or a peptide selected from SEQ ID Nos: 57-193) is immobilized to a solid support (e.g., a microtiter plate well). The antigen-specific antibody is subsequently detected using purified antigen and an enzyme-conjugated antibody specific for the antigen.

In an alternative embodiment, an “indirect ELISA” is used. In one embodiment, an antigen (or antibody) is immobilized to a solid support (e.g., a microtiter plate well) as in the direct ELISA, but is detected indirectly by first adding an antigen-specific antibody (or antigen), then followed by the addition of a detection antibody specific for the antibody that specifically binds the antigen, also known as “species-specific” antibodies (e.g., a goat anti-rabbit antibody), which are available from various manufacturers known to those in the art.

In other embodiments, a “sandwich ELISA” is used, where the antigen (e.g. contained in a test sample) is immobilized on a solid support (e.g., a microtiter plate) via an antibody (i.e., a capture antibody) that is immobilized on the solid support and is able to bind the antigen of interest. Following the affixing of a suitable capture antibody to the immobilized phase, a sample is then added to the microtiter plate well, followed by washing. If the antigen of interest is present in the sample, it is bound to the capture antibody present on the support. In some embodiments, a sandwich ELISA is a “direct sandwich” ELISA, where the captured antigen is detected directly by using an enzyme-conjugated antibody directed against the antigen. Alternatively, in other embodiments, a sandwich ELISA is an “indirect sandwich” ELISA, where the captured antigen is detected indirectly by using an antibody directed against the antigen, which is then detected by another enzyme-conjugated antibody which binds the antigen-specific antibody, thus forming an antibody-antigen-antibody-antibody complex. Suitable reporter reagents are then added to detect the third antibody. Alternatively, in some embodiments, any number of additional antibodies are added as necessary, in order to detect the antigen-antibody complex. In some preferred embodiments, these additional antibodies are labelled or tagged, so as to permit their visualization and/or quantitation.

As used herein, the term “capture antibody” refers to an antibody that is used in a sandwich ELISA to bind (i.e., capture) an antigen in a sample prior to detection of the antigen. For example, in some embodiments, a polyclonal antibody against a polypeptide selected from SEQ ID Nos: 1 to 56 and/or a peptide selected from SEQ ID Nos: 57-193 serves as a capture antibody when immobilized in a microtiter plate well. This capture antibody binds the polypeptide and/or peptide present in a sample added to the well. In one embodiment of the present invention, biotinylated capture antibodies are used in the present invention in conjunction with avidin-coated solid support. Another antibody (i.e., the detection antibody) is then used to bind and detect the antigen-antibody complex, in effect forming a “sandwich” comprised of antibody-antigen-antibody (i.e., a sandwich ELISA).

As used herein, a “detection antibody” is an antibody which carries a means for visualization or quantitation, which is typically a conjugated enzyme moiety that typically yields a colored or fluorescent reaction product following the addition of a suitable substrate. Conjugated enzymes commonly used with detection antibodies in the ELISA include horseradish peroxidase, urease, alkaline phosphatase, glucoamylase and β-galactosidase. In some embodiments, the detection antibody is directed against the antigen of interest, while in other embodiments, the detection antibody is not directed against the antigen of interest. In some embodiments, the detection antibody is an anti-species antibody. Alternatively, the detection antibody is prepared with a label such as biotin, a fluorescent marker, or a radioisotope, and is detected and/or quantitated using this label.

As used herein, the terms “reporter reagent,” “reporter molecule,” “detection substrate” and “detection reagent” are used in reference to reagents which permit the detection and/or quantitation of an antibody bound to an antigen. For example, in some embodiments, the reporter reagent is a colorimetric substrate for an enzyme that has been conjugated to an antibody. Addition of a suitable substrate to the antibody-enzyme conjugate results in the production of a colorimetric or fluorimetric signal (e.g., following the binding of the conjugated antibody to the antigen of interest). Other reporter reagents include, but are not limited to, radioactive compounds. This definition also encompasses the use of biotin and avidin-based compounds (e.g., including but not limited to neutravidin and streptavidin) as part of the detection system.

As used herein, the term “signal” is used generally in reference to any detectable process that indicates that a reaction has occurred, for example, binding of antibody to antigen. It is contemplated that signals in the form of radioactivity, fluorimetric or colorimetric products/reagents will all find use with the present invention. In various embodiments of the present invention, the signal is assessed qualitatively, while in alternative embodiments, the signal is assessed quantitatively.

As used herein, the term “amplifier” is used in reference to a system which enhances the signal in a detection method, such as an ELISA (e.g., an alkaline phosphatase amplifier system used in an ELISA).

Polypeptides of SEQ ID Nos: 1 to 56

Polypeptide Having Sequence Set Forth in SEQ ID NO: 1

This polypeptide, known as aggrecan 1, is encoded by the gene AGC1 which has been localized to 15q26. Aggrecan is an integral part of the extracellular matrix (ECM) in cartilaginous tissue and is released into bodily fluids when cleaved by aggrecanase or the like. Release of fragments of aggrecan enables measurement in synovial fluid, serum and urine. Peptides derived from aggrecan 1 are set forth as SEQ ID NOs: 57-59.

Polypeptide Having Sequence Set Forth in SEQ ID NO: 2

This polypeptide, known as asporin (ASPN), is a member of the leucine-rich repeat family of proteins associated with the cartilage matrix. Asporin contains a putative propeptide, four amino-terminal cysteines, 10 leucine-rich repeats and two carboxy-terminal cysteines.

Polypeptide Having Sequence Set Forth in SEQ ID NO: 3

This polypeptide, known as butyrylcholinesterase (BCHE) (or serum cholinesterase), is similar to the neuronal acetylcholinesterase. Mutant alleles at the BCHE locus are responsible for suxamethonium sensitivity manifested by persistent apnea following administration of the muscle relaxant during surgical anesthesia. Peptides derived from BCHE are set forth as SEQ ID NOs: 159 and 160.

Polypeptide Having Sequence Set Forth in SEQ ID NO: 4

This polypeptide, known as bone gamma-carboxyglutamate protein (BGLAP) or osteocalcin, is secreted by osteoblasts and thought to play a role in mineralization and calcium ion homeostasis.

Polypeptide Having Sequence Set Forth in SEQ ID NO: 5.

This polypeptide, known as BGN or biglycan, is a small leucine-rich repeat proteoglycan found in ECM tissues such as bone and cartilage. Biglycan consists of a core containing leucine-rich repeat regions and two glycosaminoglycan chains consisting of either chondroitin sulfate or dermatan sulfate. Biglycan appears to play a role in the mineralization of bone. Biglycan is thought to be involved in regulation of matrix assembly growth factor activity due to its ability to bind to TGFβ1. Peptides derived from BGN are set forth as SEQ ID NOs: 184-191.

Polypeptide Having Sequence Set Forth in SEQ ID NO: 6

This polypeptide, known as BMX, is a non-receptor tyrosine kinase. The BMX gene is a member of the BTK/ITK/TEC/TXK family located in chromosome Xp22.2.

Polypeptide Having Sequence Set Forth in SEQ ID NO: 7

This polypeptide, termed Ucma (unique cartilage matrix-associated protein), is a secreted cartilage-specific protein, highly conserved across species, but with no homology to other known proteins. Ucma is encoded by chrosome 10 open reading frame 49 (c10orf49).

Polypeptide Having Sequence Set Forth in SEQ ID NO: 8

This polypeptide, termed CALU or calumenin, is a calcium-binding protein located in the endo/sarcoplasmic reticulum of mammalian heart and other tissues. It is an endoplasmic reticulum chaperone protein, involved in protein folding and sorting. Calumenin is a member of the CERC EF-hand superfamily. The human and mouse CALU proteins are 98% identical. CALU and RCN3 are co-regulated; accordingly, a level of expression detected for CALU provides a measure of the level of expression of CALU or a peptide derived therefrom.

Polypeptide Having Sequence Set Forth in SEQ ID NO: 9

This polypeptide, termed cartilage paired-class homeoprotein 1 (CART1), also known as ALX1, is a member of tumor necrosis factor receptor-associated protein family. CART1 is selectively expressed in chondrocytes during embryonic development. The function of CART1 is humans has not yet been determined; however, in rodents, mutations in CART1 lead to neural tube defects.

Polypeptide Having the Sequence Set Forth in SEQ ID NO: 10

This polypeptide, termed chondroadherin (or CHAD), is a cartilage matrix protein thought to mediate adhesion of isolated chondrocytes. Chondroadherin contains 11 leucine-rich repeats flanked by cysteine-rich regions. CHAD is co-regulated with OGN and EPYC. Accordingly, a level of expression detected for CHAD provides a measure of the level of expression of OGN and/or EPYC.

Polypeptide Having the Sequence Set Forth in SEQ ID NO: 11

This polypeptide, termed chitinase 3-like 1 (CHI3L1), also known as cartilage glycoprotein-39, is a major secreted protein of ex vivo cultured articular chondrocytes and synovial cells. Chitinase 3-like 1 is a chitin-binding lectin which most likely functions in remodeling or degradation of ECM. Peptides derived from CHI3L1 are set forth as SEQ ID NOs: 161-168.

Polypeptide Having Sequence Set Forth in SEQ ID NO: 12

This polypeptide, termed cartilage intermediate layer protein (CILP) was identified and purified from human articular cartilage. The C-terminal 460 amino acids of the protein show 90% similarity to the pig ectonucleotide pyrophosphohydrolase NTPPHase. Peptides derived from CILP are set forth as SEQ ID NOs: 60-72.

Polypeptide Having Sequence Set Forth in SEQ ID NO: 13

This polypeptide, termed CILP2, is an isoform of CILP which is 50.6% identical. Peptides derived from CILP2 are set forth as SEQ ID NOs: 73-87.

Polypeptide Having Sequence Set Forth in SEQ ID NO: 14

This polypeptide, termed C-type lectin domain family 3 member A (CLEC3A), is an ECM structural constituent.

Polypeptide Having Sequence Set Forth in SEQ ID NO: 15

This polypeptide, termed Collection sub-family member 12 (COLEC12), is a member of the C-lectin family, the members of which possess collagen-like sequences and carbohydrate recognition domains. COLEC12 is a cell surface glycoprotein which can bind to carbohydrate antigens on microorganisms facilitating recognition/removal. COLEC12 may be involved in selective clearance of specific desialylated glycoproteins from circulation. Peptides derived from COLEC12 are set forth as SEQ ID NOs: 90-99.

Polypeptide Having Sequence Set Forth in SEQ ID NO: 16

This polypeptide, termed cartilage acidic protein 1 (CRTAC1), is a glycosylated ECM molecule of human articular cartilage secreted by chondrocytes. CRTAC1 is predicted to have four FG-GAP repeat domains, one RGD integrin binding motif and an EGF-like calcium binding domain.

Polypeptide Having Sequence Set Forth in SEQ ID NO: 17

This polypeptide, termed cytokine-like 1 (CYTL1) is a cytokine-like protein specifically expressed in bone marrow and cord blood mononuclear cells that bear the CD34 marker. CYTL appears to regulate the chondrogenesis of mesenchymal cells.

Polypeptide Having Sequence Set Forth in SEQ ID NO: 18

This polypeptide, termed endoglin (or ENG), is a homodimeric transmembrane RGD-containing glycoprotein highly expressed by endothelial cells. Endoglin is a component of the TGFβ receptor complex. Mutations in endoglin produce hereditary hemorrhagic telangiectasia. There are two isoforms of endoglin—SEQ ID NO: 18 is the longer of the two.

Polypeptide Having Sequence Set Forth in SEQ ID NO: 19

This polypeptide, termed epiphycan (EPYC), or dermatan sulfate proteoglycan 3, is a member of the small leucine-rich repeat proteoglycan family. Epiphycan regulates fibrillogenesis by interacting with collagen fibrils and other ECM proteins. EPYC is co-regulated with OGN and CHAD. Accordingly, a level of expression detected for EPYC provides a measure of the level of expression of OGN and/or CHAD.

Polypeptide Having Sequence Set Forth in SEQ ID NO: 20

This polypeptide, termed ethanolamine kinase 1 (ETNK1), functions in the first committed step of the phosphatidylethanolamine synthesis pathway. ETNK1 may be associated with prostatic neoplasms and seminoma. There are two distinct isoforms of ETNK—SEQ ID NO: 20 is the longer of the two.

Polypeptide Having Sequence Set Forth in SEQ ID NO: 21

This polypeptide, termed fibronectin leucine-rich transmembrane protein (FLRT) 2, may function in cell adhesion and/or receptor signaling. Peptides derived from FLRT2 are set forth as SEQ ID NOs: 100-106.

Polypeptide Having Sequence Set Forth in SEQ ID NO: 22

This polypeptide, termed FLRT3, is a member of the fibronectin leucine-rich transmembrane protein family. FLRT3 shares 44% amino acid sequence identity with FLRT2. Peptides derived from FLTR3 are set forth as SEQ ID NOs: 107-108.

Polypeptide Having Sequence Set Forth in SEQ ID NO: 23

This polypeptide, termed hyaluronan and proteoglycan link protein (HAPLN1), may function as a stabilizer of the interaction between aggrecan and hyaluronan in cartilage.

Polypeptide Having Sequence Set Forth in SEQ ID NO: 24

This polypeptide, termed IGF-like family member 3 (IGFL3), plays critical roles in cellular energy metabolism and growth and development.

Polypeptide Having Sequence Set Forth in SEQ ID NO: 25

This polypeptide, termed KIAA0999, also known as serine/threonine-protein kinase QSK, is a human cAMP-dependent protein kinase beta-catalytic subunit. QSK catalyzes the transfer of a phosphate from ATP to a protein. QSK belongs to the CAMK Ser/Thr protein kinase family.

Polypeptide Having a Sequence Set Forth in SEQ ID NO: 26

This polypeptide, termed LOC283951, is expected to localize to the cell membrane. Little is known about this polypeptide. A peptide derived from LOC283951 is set forth as SEQ ID NO: 127.

Polypeptide Having a Sequence Set Forth in SEQ ID NO: 27

This polypeptide, termed LOC284998, is a hypothetical protein encoded by a gene mapping to position 2q12.1 on chromosome 2.

Polypeptide Having a Sequence Set Forth in SEQ ID NO: 28

This polypeptide, termed LOC646627, is a phospholipase inhibitor encoded by a gene mapping to position 1q44 on chromosome 1.

Polypeptide Having a Sequence Set Forth in SEQ ID NO: 29

This polypeptide, termed lysyl oxidase-like 3 (LOXL3), is essential to the biogenesis and repair of connective tissue. LOXL3 is an extracellular copper-dependent amine oxidase that catalyzes the first step in the formation of crosslinks in collagens and elastin. LOXL3 is a susceptibility gene for intracranial aneurysms.

Polypeptide Having a Sequence Set Forth in SEQ ID NO: 30

This polyeptide, termed lysyl oxidase-like 4 (LOXL4), like LOXL3, is a member of the lysyl oxidase gene family. LOXL3 and LOXL4 are described in WO/2001/083702.

Polypeptide Having a Sequence Set Forth in SEQ ID NO: 31

This polypeptide, known as latent transforming growth factor beta (TGFβ) binding protein 1 (LTBP1), belongs to the LTBP family, members of which regulate the secretion and activation of TGFβ. LTBP1 targets latent complexes of TGFP to the ECM where TGFP is activated. There are two isoforms of LTBP1, the longer of which is represented by SEQ ID NO: 31. Peptides derived from LTBP1 are set forth as SEQ ID NOs: 109-114.

Polypeptide Having a Sequence Set Forth in SEQ ID NO: 32

This polypeptide, known as matrilin 1 (or MATN1), is a cartilage matrix protein and a member of the von Willebrand factor A domain containing family. Matrilin 1 is thought to be involved in the formation of filamentous networks in the ECM. The MATN1 gene is localized at 1p35 and is mainly expressed in cartilage. Along with MATN3 (see below), MATN1 is among the most up-regulated ECM proteins during chondrogenesis.

The Polypeptide Having a Sequence Set Forth in SEQ ID NO: 33

This polypeptide is known as MATN3, is a member of the von Willebrand factor A domain containing family having two von Willebrand factor A domains. It is present in the cartilage extracellular matrix.

The Polypeptide Having a Sequence Set Forth in SEQ ID NO: 34

This polypeptide, known as matrix, extracellular phosphoglycoprotein with ASARM motif (or MEPE), is an ECM phosphoglycoprotein and a member of the small integrin binding ligand N-linked glycoprotein (SIBLING) family. MEPE is predominantly expressed by osteocytes and is involved in phosphate and bone metabolism. MEPE promotes renal phosphate excretion and inhibits bone mineralization. A peptide derived from MEPE is set forth as SEQ ID NO: 134.

The Polypeptide Having Sequence Set Forth in SEQ ID NO: 35

This polypeptide, termed matrix Gla protein (or MGP), associates with the organic matrix of bone and cartilage and is thought to act as an inhibitor of bone formation.

The Polypeptide Having Sequence Set Forth in SEQ ID NO: 36

This polypeptide, known as matrix-remodeling associated 5 (MXRA5), also called adlican, contains leucine-rich repeat and immunoglobulin domains. MXRA5 is described in U.S. Pat. No. 7,094,890, the contents of which are incorporated herein by reference. Peptides derived from MXRA5 are set forth as SEQ ID Nos: 169-180.

The Polypeptide Having Sequence Set Forth in SEQ ID NO: 37

This polypeptide, known as matrix-remodeling-associated protein 8 (MXRA8) or limitrin, may play a role in the maturation and maintenance of blood-brain barrier, as the murine ortholog has been demonstrated to localize selectively to glia limitans in the mouse brain. Peptides derived from MXRA8 are set forth as SEQ ID Nos: 135-154.

The Polypeptide Having a Sequence Set Forth in SEQ ID NO: 38

This polypeptide, known as nidogen 2 (NID2) or osteonidogen, is a component of the basement membrane. Lack of nidogen 1 and nidogen 2 prevents basement membrane assembly in vitro. Nidogen 2 exhibits aberrant methylation in several human cancers. A peptide derived from NID2 is set forth as SEQ ID No: 88.

The Polypeptide Having a Sequence Set Forth in SEQ ID NO: 39

This polypeptide, known as NLRP5 or NALP5, is a member of the NALP protein family, originally identified as an oocyte specific antigen in mice. Members of this family typically contain a NACHT domain, a NACHT-associated domain (NAD), a C-terminal leucine-rich repeat (LRR) region, and an N-terminal pyrin domain (PYD). NALP5 appears to be a tissue-specific autoantigen involved in hypoparathyroidism in patients with APS-1. U.S. Patent Publication No. 20040248775 discloses that NALP5 expression is elevated after transient cerebral artery occlusion.

The Polypeptide Having a Sequence Set Forth in SEQ ID NO: 40

This polypeptide, known as nephronectin (or NPNT) is a human epidermal growth factor-like ECM protein expressed in a number of embryonic and adult tissues, including kidney, and lung. The gene encoding NPNT is located at chromosomal position 4q25. Peptides derived from NPNT are set forth as SEQ ID Nos: 115-126.

The Polypeptide Having a Sequence Set Forth in SEQ ID NO: 41

This polypeptide, known as osteoglycin (OGN), is a small proteoglycan which contains tandem leucine-rich repeats. Osteoglycin induces ectopic bone formation in conjunction with TGFβ. Altered expression of osteoglycin has been correlated with enlarged hearts, epecially left ventricular hypertrophy. Peptides derived from OGN are set forth as SEQ ID Nos: 130-133. OGN is co-regulated with CHAD and EPYC; accordingly, a level of expression detected for OGN or a peptide derived therefrom provides a measure of the level of expression of CHAD and/or EPYC.

The Polypeptide Having a Sequence Set Forth in SEQ ID NO: 42

This polypeptide, known as osteomodulin/osteoadherin (OMD) is a cell-binding keratin sulfate proteoglycan belonging to the leucine-rich proteoglycan family. OMD contains 12 leucine-rich repeats and may be implicated in biomineralization. OMD has been shown to bind α₅β₃ integrin. Peptides derived from OMD are set forth as SEQ ID Nos: 192 and 193.

The Polypeptide Having a Sequence Set Forth in SEQ ID NO: 43

This polypeptide, known as oncostatin M receptor (OSMR), is part of a heterodimeric receptor complex (along with gp130) that mediates signal transduction of the cytokine oncostatin M, a member of the IL6 cytokine family. Mutations in OSM are associated with a form of amyloidosis. Mice deficient in OSMR exhibit a reduced number of peripheral erythrocytes and platelets relative to wild type mice.

The Polypeptide Having a Sequence Set Forth in SEQ ID NO: 44

This polyeptpide, termed osteocrin (OSTN) or musclin, is a vitamin-D regulated bone-specific protein. Osteocrin is highly expressed in osteoblasts and may function as a negative regulator of osteoblast differentiation and may also be involved in ossification. Peptides derived from OSTN are set forth as SEQ ID Nos: 128 and 129.

The Polypeptide Having a Sequence Set Forth in SEQ ID NO: 45

This polypeptide, termed periostin (POSTN) is an osteoblast specific factor which may modulate new bone formation and cell adhesion. POSTN has also been implicated as essential for cardiac healing after infarction.

The Polypeptide Having a Sequence Set Forth in SEQ ID NO: 46

This polypeptide, termed proteoglycan 4 (PRG4) is a large proteoglycan specifically synthesized by chondrocytes located at the surface of articular cartilage. PRG4 functions as a lubricant at the cartilage surface and contributes to the elastic absorption and energy dissipation of synovial fluid. PRG4 exists as multiple isoforms—SEQ ID NO 46 represents the longest of these isoforms. Peptides derived from PRG4 are set forth as SEQ ID Nos: 181-183.

The Polypeptide Having Sequence Set Forth in SEQ ID NO: 47

This polypeptide, termed pleiotrophin (PTN), also known as heparin binding growth factor 8 and neurite growth promoting factor 1, is a proangiogenic cytokine that potentiates cardiomyocyte apoptosis. PTN may participate in nervous system development, in bone mineralization and in learning. PTN may be associated with astrocytomas and brain neoplasms.

The Polypeptide Having Sequence Set Forth in SEQ ID NO: 48

This polypeptide, known as reticulocalbin 3 (RCN3), contains an EF-hand calcium binding domain. RCN3 is a member of the Cab45/reticulocalbin/ERC45/calumenin) (CREC) family of multiple EF-hand calcium-binding proteins localized to the secretory pathway. A peptide derived from RCN3 is set forth as SEQ ID NO: 89. RCN3 and CALU are co-regulated; accordingly, a level of expression detected for RCN3 or a peptide derived therefrom provides a measure of the level of expression of CALU.

The Polypeptide Having Sequence Set Forth in SEQ ID NO: 49

This polypeptide, known as sema domain, transmembrane and cytoplasmic domain (semaphorin) 6D (SEMA6D), is a member of the semaphorin family, a large protein family implicated as inhibitors or chemorepellents in axon pathfinding, branching and target selection. The SEMA6D gene encodes six identified transcripts; SEQ ID NO: 49 represents the longest of the encoded polypeptides.

The Polypeptide Having Sequence as Set Forth in SEQ ID NO: 50

This polypeptide, known as serine peptidase inhibitor, clade E (nexin, plasminogen activator inhibitor type 1) member 2 (SERPINE 2), is an extracellular serine proteinase inhibitor activity toward trypsin, thrombin, plasmin and other serine proteinases. SERPINE2 has been implicated as a chronic obstructive pulmonary disease (COPD) susceptibility gene. A peptide derived from SERPINE2 is set forth as SEQ ID NO: 155.

The Polypeptide Having Sequence as Set Forth in SEQ ID NO: 51

This polypeptide, termed solute carrier family 15, member 3 (SLC15A3), is a transporter protein likely involved in oligopeptide transport.

The Polypeptide Having Sequence as Set Forth in SEQ ID NO: 52

This polypeptide, termed solute carrier family 28 (sodium-coupled nucleoside transporter) member 3 (SLC28A3), is a member of the nucleoside transporter family, the members of which regulate multiple cellular processes including neurotransmission, vascular tone, and the transport and metabolism of nucleoside drugs.

The Polypeptide Having Sequence as Set Forth in SEQ ID NO: 53

This polypeptide, known as tubulin folding cofactor A (TBCA), is one of four proteins (cofactors A, D, E, C) involved in the pathway leading to correctly folded β-tubulin from intermediates. Peptides derived from TBCA are set forth as SEQ ID Nos: 156-158.

The Polypeptide Having Sequence as Set Forth in SEQ ID NO: 54

This polypeptide, known as twist homolog 2 (TWIST2), is a basic helix-loop-helix transcription factors which have been implicated in cell lineage determination and differneation. It is thought that during osteoblast development, TWIST2 may inhibit osteoblast maturation and maintain cells in preosteoblast phenotype. Reduced expression of TWIST2 may suppress the multistep process of peritoneal dissemination.

The Polypeptide Having Sequence as Set Forth in SEQ ID NO: 55

This polypeptide, known as LOC339316, is hypothetical protein encoded by a gene mapping to position 19q12 on chromosome 19.

The Polypeptide Having Sequence as Set Forth in SEQ ID NO: 56

This polypeptide, known as LRC15, or human leucine-rich repeat-containing protein 15 [precursor], is encoded by the LRRC15 gene mapping to position 3q29 on chromosome 3. LRC15 is a potential single-pass type 1 membrane protein of 581 amino acids.

It is to be understood that homologs of a polypeptide of SEQ ID NOs 1 to 56 may also be useful in the invention.

Antibodies

Polyclonal

In one embodiment, the present invention provides antibodies which bind to a polypeptide selected from SEQ ID Nos: 1-56 and/or a peptide selected from SEQ ID Nos: 57-193, and which may find use herein as diagnostic agents. Exemplary antibodies include polyclonal, monoclonal, humanized, bispecific, and heteroconjugate antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, or SOCl₂.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

Monoclonal

Monoclonal antibodies may be made using the hybridoma method or may be made by recombinant DNA methods.

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis.

Once hybridoma cells that produce antibodies of the desired specificity, affinity, and/or activity are identified, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, or the like.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

In a further embodiment, monoclonal antibodies or antibody fragments can be isolated from antibody phage libraries as a viable alternative to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Human and Humanized Antibodies

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody). According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody. Another method uses a particular framework region derived from the consensus sequence of all human antibodies of a particular subgroup of light or heavy chains. The same framework may be used for several different humanized antibodies.

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Several sources of V-gene segments can be used for phage display. A repertoire of V genes from unimmunized human donors can be constructed and antibodies to a diverse array of antigens (including self-antigens) can be isolated essentially following the techniques described.

Antibody Fragments

In certain circumstances there are advantages of using antibody fragments, rather than whole antibodies. The smaller size of the fragments allows for rapid clearance, and may lead to improved access to antigen(s).

Various techniques have been developed for the production of antibody fragments. Traditionally, these fragments were derived via proteolytic digestion of intact antibodies. However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments. According to another approach, F(ab′)₂ fragments can be isolated directly from recombinant host cell culture. Fab and F(ab′)₂ fragment with increased in vivo half-life comprising a salvage receptor binding epitope residues are known. Other techniques for the production of antibody fragments will be apparent to the skilled practitioner. In other embodiments, the antibody of choice is a single chain Fv fragment (scFv). Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. The antibody fragment may also be a “linear antibody.” Such linear antibody fragments may be monospecific or bispecific.

Bispecific Antibodies

Bispecific antibodies are antibodies that have binding specificities for at least two different epitopes. Exemplary bispecific antibodies may bind to two different epitopes of a polypeptide selected from SEQ ID Nos: 1 to 56 and/or a peptide selected from SEQ ID Nos: 57-193, as described herein. Other such antibodies may combine an epitope of a polypeptide selected from SEQ ID Nos: 1 to 56 and/or a peptide selected from SEQ ID Nos: 57-193 with a binding site for another protein. Bispecific antibodies can be prepared as full length antibodies or antibody fragments (e.g., F(ab′)₂) bispecific antibodies.

Methods for making bispecific antibodies are known in the art. Traditional production of full length bispecific antibodies is based on the co-expression of two immunoglobulin heavy chain-light chain pairs, where the two chains have different specificities.

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region.

Epitope Mapping

Mapping of epitopes recognized by antibodies binding, preferably specifically, to a polypeptide selected from SEQ ID Nos: 1-56 and/or a peptide selected from SEQ ID Nos: 57-193 can be performed as described in detail in “Epitope Mapping Protocols (Methods in Molecular Biology) by Glenn E. Morris ISBN-089603-375-9 and in “Epitope Mapping: A Practical Approach” Practical Approach Series, 248 by Olwyn M. R. Westwood, Frank C. Hay. In a preferred embodiment, epitope scanning is used to identify epitopes recognized by antibodies specifically binding to a polypeptide selected from SEQ ID Nos: 1-56 and/or a peptide selected from SEQ ID Nos.: 57-193. Briefly, overlapping peptides encompassing the selected polypeptide sequence are synthesized on individual plastic pins. Recognition of these peptides by antibodies against the selected polypeptide is measured, preferably by ELISA.

Oligopeptides

Oligopeptides of the present invention are oligopeptides that bind, preferably specifically, to a polypeptide selected from SEQ ID Nos: 1 to 56 and/or a peptide selected from SEQ ID Nos: 57-193 as described herein. The oligopeptides may be chemically synthesized using known oligopeptide synthesis methodology or may be prepared and purified using recombinant technology. The oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a polypeptide selected from SEQ ID Nos: 1 to 56 and/or a peptide selected from SEQ ID Nos: 57-193 as described herein. The oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles. The utility of phage display lies in the fact that large libraries of selectively randomized protein variants (or randomly cloned cDNAs) can be rapidly and efficiently sorted for those sequences that bind to a target molecule with high affinity. Display of peptide or protein libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Organic Molecules that Bind a Polypeptide Selected from SEQ ID Nos: 1 to 56 and/or a Peptide Selected from SEQ ID Nos: 57-193.

Organic molecules of the invention are organic molecules other than oligopeptides or antibodies as defined herein that bind, preferably specifically, to a polypeptide selected from SEQ ID Nos: 1 to 56 and/or a peptide selected from SEQ ID Nos: 57-193 as described herein. The organic molecules may be identified and chemically synthesized using known methodology (see, e.g., PCT Publication Nos. WO00/00823 and WO00/39585). The organic molecules are usually less than about 2000 daltons in size, alternatively less than about 1500, 750, 500, 250 or 200 daltons in size, wherein such organic molecules that are capable of binding, preferably specifically, to a polypeptide selected from SEQ ID Nos: 1 to 56 and/or a peptide selected from SEQ ID Nos: 57-193 described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585). Organic molecules of the invention may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

Kits

Kits are provided that are useful for various purposes, e.g., for the diagnosis of a bone and/or cartilage disorder or for purification or immunoprecipitation of a polypeptide selected SEQ ID Nos: 1 to 56 and/or a peptide selected from SEQ ID Nos: 57-193 from cells, tissues and/or body fluids. For isolation and purification of such polypeptides, the kit can contain a specific binding agent (i.e. an antibody, oligopeptide, or organic molecule) coupled to beads (e.g., sepharose beads). Kits can be provided which contain the antibodies, oligopeptides or organic molecules for detection and quantitation of such polypeptide in vitro, e.g., in an ELISA or a Western blot. The kit comprises a container and a label or package insert on or associated with the container. The container holds a composition comprising at least one antibody, oligopeptide or organic molecule of the invention. Additional containers may be included that contain, e.g., diluents and buffers, control antibodies. The label or package insert may provide a description of the composition as well as instructions for the intended in vitro or diagnostic use.

The present invention also provides ELISA kits for the detection of a polypeptide selected from SEQ ID Nos: 1 to 56 and/or a peptide selected from SEQ ID Nos: 57-193. In addition, in some embodiments, the kits are customized for various applications. However, it is not intended that the kits of the present invention be limited to any particular format or design. In some embodiments, the kits of the present invention include, but are not limited to, materials for sample collection (e.g., spinal and/or venipuncture needles), tubes (e.g., sample collection tubes and reagent tubes), holders, trays, racks, dishes, plates (e.g., 96-well microtiter plates), instructions to the kit user, solutions or other chemical reagents, and samples to be used for standardization, and/or normalization, as well as positive and negative controls. In particularly preferred embodiments, reagents included in ELISA kits specifically intended for the detection of a polypeptide selected from SEQ ID Nos: 1 to 56 and/or a peptide selected from SEQ ID Nos: 57-193 include a control polypeptide or peptide selected from SEQ ID Nos: 1 to 193, antibody against said polypeptide or peptide, antibody against said polypeptide or peptide conjugated to an enzyme, 96-well microtiter plates precoated with said polypeptide or peptide, suitable capture antibody, 96-well microtiter plates precoated with a suitable capture antibody against said polypeptide or peptide, buffers (e.g., coating buffer, blocking buffer, and distilled water), enzyme reaction substrate and premixed enzyme substrate solutions.

The present invention also relates to a kit comprising an ordered array of antibodies that specifically bind to polypeptides of SEQ ID Nos: 1 to 56 and/or peptides of SEQ ID Nos: 57-193 for detecting the expression of a polypeptide selected from SEQ ID Nos: 1 to 56 and/or a peptide selected from SEQ ID Nos: 57-193 in a sample, comprising one or more antibodies associated with a solid support, wherein each antibody is specific for a polypeptide of SEQ ID Nos: 1 to 56 and/or a peptide of SEQ ID Nos: 57-193.

The phrase “ordered array” indicates that the probes are arranged in an identifiable or position-addressable pattern, e.g. such as the arrays disclosed in U.S. Pat. Nos. 6,156,501 and 6,077,673. The probes or antibodies are associated with the solid support in any effective way. For example, the probes can be bound to the solid support either by polymerizing the probes on the substrate or by attaching a probe to the substrate. Association can be covalent, noncovalent, electrostatic, hydrophobic, hydrophilic, adsorbed, absorbed, polar, etc.

Methods

Methods of Detecting Polypeptides

Polypeptides of SEQ ID Nos: 1 to 56 and/or peptides of SEQ ID Nos: 57-193 can be detected, visualized, determined, quantitated, etc. according to any effective method. Useful methods include, but are not limited to, immunoassays, radioimmunoassay (RIA), ELISA, immunofluorescence, flow cytometry, histology, electron microscopy, light microscopy, in situ assays, immunoprecipitation, Western blot and the like.

Immunoassays may be carried out in liquid or on a support. For instance, a sample (e.g. blood, urine, tissue, body fluids, etc.) can be brought in contact with and immobilized onto a solid phase support or carrier such as nitrocellulose or other solid support capable of immobilizing cells, cell particles or soluble proteins. The support may then be washed with suitable buffers followed by treatment with detectably labeled antibody specifically recognizing a polypeptide of SEQ ID Nos: 1 to 56 and/or a peptide of SEQ ID Nos: 57-193. The solid phase support can then be washed with a buffer a second time to remove unbound antibody. The amount of bound label on the solid support may then be detected by conventional means.

Diagnosis of a Bone and/or Cartilage Disorder

Yet another embodiment of the present invention is directed to a method of diagnosing a bone and/or cartilage disorder, or a susceptibility to a bone and/or cartilage disorder, in a mammal, which is based on the altered expression of a polypeptide selected from SEQ ID Nos: 1 to 56, or a peptide selected from SEQ ID Nos: 57-193 derived therefrom, will provide a valuable clinical marker correlated with such a disorder.

Such methods comprise determining if a polypeptide selected from SEQ ID Nos: 1 to 56, or a peptide of SEQ ID Nos: 57-193 derived therefrom, is overexpressed or underexpressed in a test sample as compared to a normal sample. Polypeptides of SEQ ID Nos: 1 to 56 are selectively expressed in human bone and cartilage tissue, as shown in the examples of this application. Peptides of SEQ ID Nos: 57-193 are derived from polypeptides of SEQ ID Nos: 1 to 56. An increased or decreased presence of a polypeptide selected from SEQ ID Nos: 1 to 56, or a peptide selected from SEQ ID Nos: 57-193 derived therefrom, in a patient-derived sample is carried out using any standard methodology that measures levels (as compared to known normal controls) of a certain protein, e.g., by Western blot assays or a quantitative assay such as ELISA. For example, a standard competitive ELISA format using an antibody specific for a polypeptide selected from SEQ ID Nos: 1 to 56, or a peptide selected from SEQ ID Nos: 57-193, is used to quantify levels of the polypeptide. Alternatively, a sandwich ELISA using a first antibody as the capture antibody and a second antibody specific for a polypeptide or peptide selected from SEQ ID Nos: 1 to 193 as a detection antibody is used.

In one embodiment, the method comprises (a) obtaining a test sample from a patient suspected of having a bone and/or cartilage disorder (b) detecting an expression level of a polypeptide selected from the group consisting of SEQ ID NOs 19 (EPYC), 27 (LOC284998), 38 (NID2), 39 (NLRP5), 41 (OGN) and 55 (LOC339316), by assaying for a polypeptide selected from the group consisting of SEQ ID NOs: 19, 27, 38, 39, 41 and 55; and (c) comparing said level to that of a healthy control, whereby an alteration in the expression level of a polypeptide selected from the group consisting of SEQ ID NOs: 19, 27, 38, 39, 41 and 55 relative to the level of expression of a polypeptide selected from the group consisting of SEQ ID NOs: 19, 27, 38, 39, 41 and 55 in the control, indicates a positive result for a bone and/or a cartilage disorder.

In another embodiment, the method comprises (a) obtaining a test sample from a patient suspected of having a bone and/or cartilage disorder (b) detecting an expression level of a peptide selected from the group consisting of SEQ ID NOs: 88, 89, 130, 131, 132, 133, 127, 107 and 108, by assaying for said peptide; and (c) comparing said level to that of a healthy control, whereby an alteration in the expression level of a peptide selected from the group consisting of SEQ ID NOs: 88, 89, 130, 131, 132, 133, 127, 107 and 108 relative to the level of expression of the same peptide in the control, indicates a positive result for a bone and/or cartilage disorder.

For diagnostic purposes, the specific binding agents (i.e. antibodies, oligopeptides or small organic molecules) of the present invention may be detectably labeled, attached to a solid support, or the like. The nature of the solid surface may vary depending on the assay format. For assays carried out in microtiter wells, the solid surface is the wall of the well or cup. For assays using beads, the solid surface is the surface of the bead. Examples of useful solid supports include nitrocellulose (e.g. in membrane or microtiter well form), polyvinyl chloride (e.g. in sheets or microtiter wells), polystyrene latex (e.g. in beads or microtiter plates), polyvinylidine fluoride, diazotized paper, nylon membranes, activated beads and Protein A beads. The solid support containing the specific binding agent is typically washed after contacting it with the test sample, and prior to detection of bound complexes. Incubation of the specific binding agent with the test sample is followed by detection of complexes by a detectable label. For example, the label is enzymatic, fluorescent, chemiluminescent, radioactive or a dye. Assays which amplify the signals from the complex are also known in the art, e.g. assays which utilize biotin and avidin.

In preferred embodiments of the present invention, ELISA methods for quantitation of a polypeptide selected from SEQ ID Nos: 1 to 56 or a peptide selected from SEQ ID Nos: 57-193 (the antigen) in a test sample are provided. In some of these methods, the antigen is first immobilized on a solid support (e.g. in a microtiter plate well). Detection and quantitation of the immobilized antigen is accomplished by use of an antibody-enzyme conjugate capable of binding to the immombilized antigen and producing a quantifiable signal. In some embodiments, the amount of antigen present is directly proportional to the amount of enzyme reaction product produced after the addition of an appropriate enzyme substrate.

The end product of an ELISA is a signal typically observed as the development of color or fluorescence. Typically, this signal is read (i.e., quantitated) using a suitable spectrocolorimeter (i.e. a spectrophotometer) or spectrofluorometer. The amount of color or fluorescence is directly proportional to the amount of immobilized antigen. In some embodiments of the present invention, the amount of antigen in a sample (e.g. the amount of a polypeptide or peptide selected from SEQ ID Nos: 1 to 193 in a synovial fluid or blood sample) is quantitated by comparing results obtained for the sample with a series of control wells containing known concentrations of the antigen (i.e. a standard concentration curve). A negative control is also included in the assay system.

The present invention provides various ELISA protocols for the detection and/or quantitation of a polypeptide or peptide selected from SEQ ID Nos: 1 to 193 in a sample. In one embodiment, the present invention provides a “direct ELISA” for the detection of a polypeptide or peptide selected from SEQ ID Nos: 1 to 193 in a sample. In some embodiments, the antigen of interest in a sample (i.e. a polypeptide or peptide selected from SEQ ID Nos: 1 to 193) is bound (along with unrelated antigens) to the solid support. The immobilized antigen is then directly detected by an antigen-specific enzyme-conjugated antibody. Addition of an appropriate detection substrate results in color development or fluorescence that is proportional to the amount of antigen present in the well.

In another embodiment, the present invention provides an indirect ELISA for the detection of antigen in a sample. In this embodiment, antigen of interest in a sample is immobilized (along with unrelated antigens) to a solid support as in the direct ELISA but is detected indirectly by first adding an antigen-specific antibody, then followed by the addition of a detection antibody specific for the antibody that specifically binds the antigen, also known as “species-specific” antibodies (e.g. a goat anti-rabbit antibody).

In some embodiments, the concentration of sample added to each well is titrated so as to produce an antigen concentration curve. In other embodiments, the concentration of conjugated antibody is titrated. Indeed, such titrations are typically performed during the initial development of ELISA systems.

In another embodiment, the present invention provides “sandwich ELISA” methods, in which the antigen in a sample is immobilized on the solid support by a “capture antibody” that has been previously bound to the solid support. In general, the sandwich ELISA method is more sensitive than other configurations, and is capable of detecting 0.1-1.0 ng/ml protein antigen. As indicated above, the sandwich ELISA method involves pre-binding the “capture antibody” which recognizes the antigen of interest (i.e., a polypeptide or peptide selected from SEQ ID Nos: 1 to 193) to the solid support (e.g., wells of the microtiter plate). In some embodiments, a biotinylated capture antibody is used in conjunction with avidin-coated wells. Test samples and controls are then added to the wells containing the capture antibody. If antigen is present in the samples and/or controls, it is bound by the capture antibody.

In some embodiments, after a washing step, detection of antigen that has been immobilized by the capture antibody is detected directly (i.e., a direct sandwich ELISA). In other embodiments detection of antigen that has been immobilized by the capture antibody is detected indirectly (i.e., an indirect sandwich ELISA). In the direct sandwich ELISA, the captured antigen is detected using an antigen-specific enzyme-conjugated antibody. In the indirect sandwich ELISA, the captured antigen is detected by using an antibody directed against the antigen, which is then detected by another enzyme-conjugated antibody which binds the antigen-specific antibody, thus forming an antibody-antigen-antibody-antibody complex. In both the direct and indirect sandwich ELISAs, addition of a suitable detection substrate results in color development or fluorescence that is proportional to the amount of antigen that is present in the well.

It is not intended that the present invention be limited to the direct ELISA and sandwich ELISA protocols particularly described herein, as the art knows well numerous alternative ELISA protocols that also find use in the present invention (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; and Ausubel et al. (eds.), Current Protocols in Molecular Biology, Ch. 11, John Wiley & Sons, Inc., New York [1994]). Thus, any suitable ELISA method including, but not limited to, competitive ELISAs also find use with the present invention. Similarly, it is not intended that detection methods be limited to ELISA methods, and the art knows well numerous alternative detection methods that also find use in the present invention including, but not limited to, immunofluorescence, flow cytometry, histology, electron microscopy, light microscopy, in situ assays, immunoprecipitation and Western blot.

All of the references discussed herein are incorporated by reference in their entirety.

The following Examples are meant to be illustrative of the invention and are not intended to limit the scope of the invention as set out is the appended claims.

### EXAMPLE 1

### Identification of Polypeptides Set Forth as SEQ ID Nos: 1 to 56 as Selectively Expressed in Bone and/or Cartilage Tissue

Procedures for the mining of databases containing information about gene expression in human tissues were developed. Genes selectively expressed in cartilage or bone tissue were identified by analyzing transcriptomes. The sources of the transcriptomes were public databases, in particular the UniGene and GEO databases.

The first three tasks were: (1) updating transcriptomes of human tissues for use in the MPW Analyzer, a bioinformatics mining tool that uses gene symbols to connect entries in databases (genes, transcripts, proteins and peptides) with gene transcripts (2) importing animal genomics resources into the data warehouse and (3) assessing the suitability of the transcriptomes for identification of tissue-specific proteins. Transcriptomes of tissues represented in databases were deemed to be of sufficient quality for analysis of tissue-selective expression of genes if enzymes and protein iso forms are well represented in twenty metabolic domains (e.g. amino acid metabolism; aromatic compound metabolism; one-carbon metabolism; carbhohydrate metabolism; coenzymes and vitamins; electron transport; enzyme metabolism; exogenous compounds; lipid metabolism; membrane transport; nucleic acid metabolism; oxygen metabolism; phosphorus metabolism; protein metabolism; purine metabolism; pyrimidine metabolism; signal transduction; and sulfur metabolism).

Transcriptomes of human tissues of sufficient quality for a systematic investigation of tissue-selective transcription were identified. That is, transcript levels could be assigned a number for selective expression in one tissue relative to forty-one other tissues, while correcting for false positives in the tissue samples. This number, the RD value, is RD=(1−(TPMb/TPMa)), wherein TPM is the number of transcripts per million transcripts. In this formula, TPMa is the TPM for the gene of interest in the tissue having the highest expression level (here bone or cartilage tissue), and TPMb is the TPM for the same gene in the tissue with the next highest expression level for that gene. Thus, RD provides a measure of the selectivity of expression of the gene of interest in cartilage or bone tissue. RD has a value between 0 and 1, with 1 being the highest possible RD value (corresponding to the highest selectivity of expression). Preferred polypeptides for use in the methods of the present invention are polypeptides encoded by genes with RD values≧0.8.

Over one hundred mammalian genes in the first instance were found to be expressed with high selectivity in cartilage, bone or the vasiculature, or combinations thereof. Of these, half are human genes. From these some human proteins were selected with priority. Criteria such as tissue-specificity of expression, occurrence of a gene product in body fluids, occurrence of veterinary homologues, low number of interactions with extracellular matrix (ECM) proteins, and undesired structural properties of the proteins were used in the selection procedure. Of these selected proteins, some represent hypothetical proteins (“unknowns”), some are highly selective for bone, some for cartilage and only a few for the vasculature. Of the selected proteins, only a few are expressed intracellularly; the remainder are secreted and are minor components of the ECM. Most selected proteins have congeners in veterinary animals. The amino acid sequences of the selected protein are set forth as SEQ ID Nos: 1 to 56 SEQ ID Nos: 1 to 56 include a set of proteins (CHAD, OMD, OGN, ASPN, CALU and RCN3) that is presumably co-regulated via TGF-β and Runx2. These proteins may be assayed for singly or in combinations, for example by antibodies against peptide(s) derived from one of the members.

The RD values for polypeptides of SEQ ID Nos: 1 to 56 are provided in Table 1. Body fluid peptides derived from polypeptides of SEQ ID NOs: 1 to 56 are also listed in Table 1.

TABLE 1

| | SEQ | RD VALUE | |
|---|---|---|---|
| POLYPEPTIDE | ID # | (RANGE) | BODY FLUID PEPTIDES |
| AGC1 | 1 | 0.9-1.0 | SEQ ID Nos: 57-59 |
| ASPN | 2 | 0.8-0.9 | |
| BCHE | 3 | 0.8-0.9 | SEQ ID Nos: 159-160 |
| BGN | 5 | 0.6-0.7 | SEQ ID Nos: 184-191 |
| BMX | 6 | 0.9-1.0 | |
| C10orf49 | 7 | 0.9-1.0 | |
| CALU | 8 | 0.7-0.8 | |
| CHAD | 10 | 0.8-0.9 | |
| CHI3L1 | 11 | 0.7-0.8 | SEQ ID Nos: 161-168 |
| CILP | 12 | 0.8-0.9 | SEQ ID Nos: 60-72 |
| CILP2 | 13 | 0.8-0.9 | SEQ ID Nos: 73-87 |
| CLEC3A | 14 | 0.6-0.7 | |
| COLEC12 | 15 | 0.7-0.8 | SEQ ID Nos: 90-99 |
| CRTAC1 | 16 | 0.8-0.9 | |
| CYTL1 | 17 | 0.9-1.0 | |
| EPYC | 19 | 0.8-0.9 | |
| ETNK1 | 20 | 0.8-0.9 | |
| FLRT2 | 21 | 0.7-0.8 | SEQ ID Nos: 100-106 |
| FLRT3 | 22 | 0.7-0.8 | SEQ ID Nos: 107-108 |
| HAPLN1 | 23 | 0.7-0.8 | |
| IGFL3 | 24 | 0.6-0.7 | |
| KIAA0999 | 25 | 0.6-0.7 | |
| LOC283951 | 26 | 0.6-0.7 | SEQ ID NO: 127 |
| LOC284998 | 27 | 0.9-1.0 | |
| LOC339316 | 55 | 0.7-0.8 | |
| LOC646627 | 28 | 0.8-0.9 | |
| LOXL4 | 30 | 0.6-0.7 | |
| LTBP1 | 31 | 0.7-0.8 | SEQ ID NOs: 109-114 |
| MATN1 | 32 | 0.9-1.0 | |
| MATN3 | 33 | 0.7-0.8 | |
| MEPE | 34 | 0.8-0.9 | SEQ ID NO: 134 |
| MXRA5 | 36 | 0.5-0.6 | SEQ ID NOs: 169-180 |
| MXRA8 | 37 | 0.7-0.8 | SEQ ID NOs: 135-154 |
| NID2 | 38 | 0.8-0.9 | SEQ ID NO: 88 |
| NLRP5 | 39 | 0.9-1.0 | |
| NPNT | 40 | 0.7-0.8 | SEQ ID NOs: 115-126 |
| OGN | 41 | 0.9-1.0 | SEQ ID NOs: 130-133 |
| OMD | 42 | 0.6-0.7 | SEQ ID NOs: 192-193 |
| OSMR | 43 | 0.6-0.7 | |
| OSTN | 44 | >0.8 | SEQ ID NOs: 128-129 |
| POSTN | 45 | 0.7-0.8 | |
| PRG4 | 46 | 0.8-0.9 | SEQ ID NOs: 181-183 |
| PTN | 47 | 0.8-0.9 | |
| RCN3 | 48 | 0.7-0.8 | SEQ ID NO: 89 |
| SEMA6D | 49 | 0.6-0.7 | |
| SERPINE2 | 50 | 0.7-0.8 | SEQ ID NO: 155 |
| SLC28A3 | 51 | 0.7-0.8 | |
| TBCA | 53 | 0.6-0.7 | SEQ ID NOs: 156-158 |
| TWIST2 | 54 | 0.5-0.6 | |
| LRC15 | 56 | 0.9-1.0 | |

### EXAMPLE 2

### Testing for Altered Expression of a Polypeptide Selected From SEQ ID NOs: 1 to 56 or a Peptide Selected From SEQ ID NOs: 57 to 193 in a Bone and/or Cartilage Disease

Antibodies may be utilized in accordance with the present invention to detect altered expression of a polypeptide of SEQ ID NOs: 1 to 56 and/or a peptide of SEQ ID NOs: 57-193 in a bone and/or cartilage disorder. According to this procedure, an appropriate sample from a patient having a bone and/or cartilage disorder and a sample from a healthy age- and gender-matched control are collected. The samples are then added to an ELISA, as described above, containing antibodies which specifically bind one or more polypeptides and/or peptides of SEQ ID NOs: 1 to 193. The amount of binding is then quantified and compared.

It is to be understood that other techniques known in the art for measuring the expression level of proteins, including but not limited to, protein array, mass spectroscopy, gel electrophoresis and microarray immunoassay, may also be used to determine altered expression of a polypeptide or peptide selected from the group consisting of SEQ ID NOs: 1 to 193.

### EXAMPLE 3

### Correlation of Marker Concentration with Clinical Status of Patients Suffering from a Bone or Cartilage Disorder

In order to examine whether each of the polypeptides of SEQ ID NOs: 1-56 (i.e., the “targets”) is a suitable biomarker of a bone or cartilage disorder, the concentration of each target was correlated with clinical status of patients suffering from a bone (e.g. osteoporosis) or cartilage (e.g. rheumatoid arthritis) disorder. To this end, the concentration of each target under investigation was aligned with clinical data or scores defined by the doctors treating those patients or other established biomarkers. For example, the “Larson Score” is a radiological defined value estimating the extent of cartilage degradation caused by arthritis. The proof of clinical concept for a given target in this case is the correlation with the Larson Score, i.e., that elevated or lowered levels of the target predict cartilage degradation.

The research consisted of (1) amino acid/epitope analysis of the targets for defining immunogens to be used to generate antibodies (2) immunizations with the synthetic peptides defined by epitope anaylsis in order to generate antibodies and characterization of the obtained antisera and (3) clinical evaluation of the targets in which suitable immunological screening assays using the generated antisera were developed and well defined clinical serum samples from osteoporotic or arthritic patients were tested using the assays. Each polypeptide or peptide of SEQ ID NOs: 1-56 was investigated.

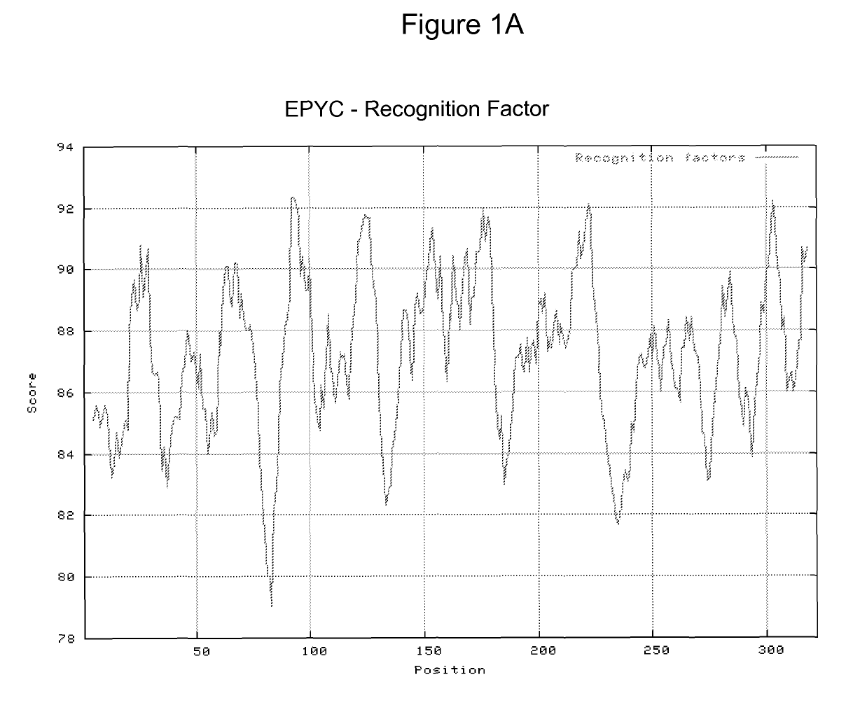

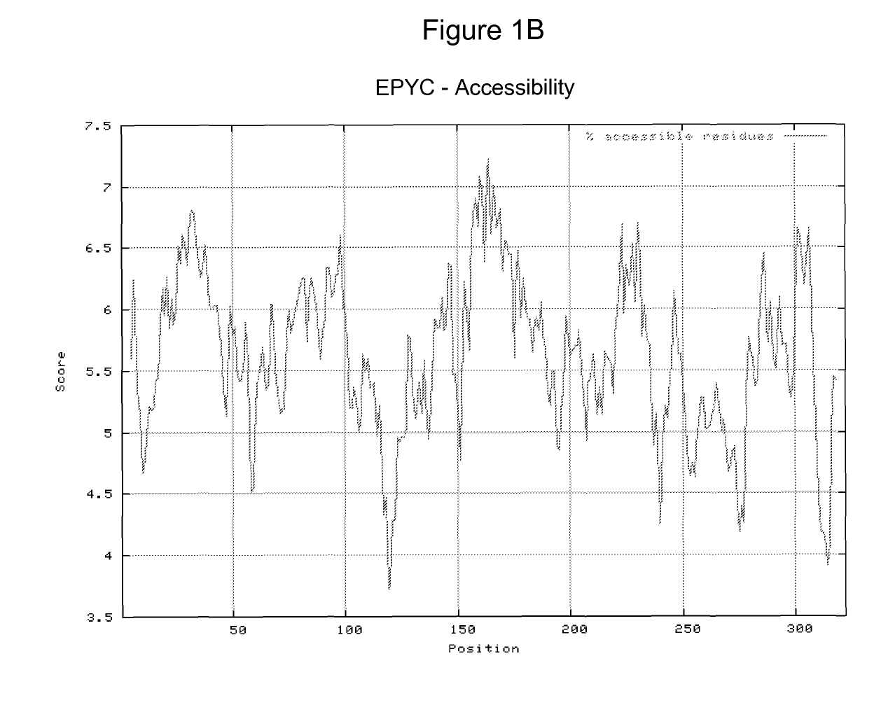

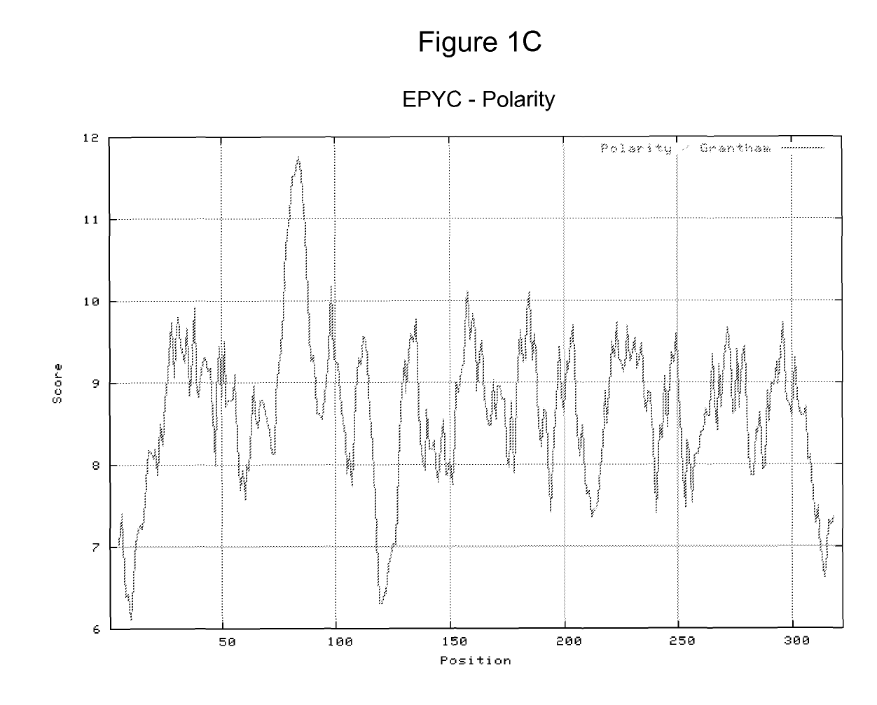

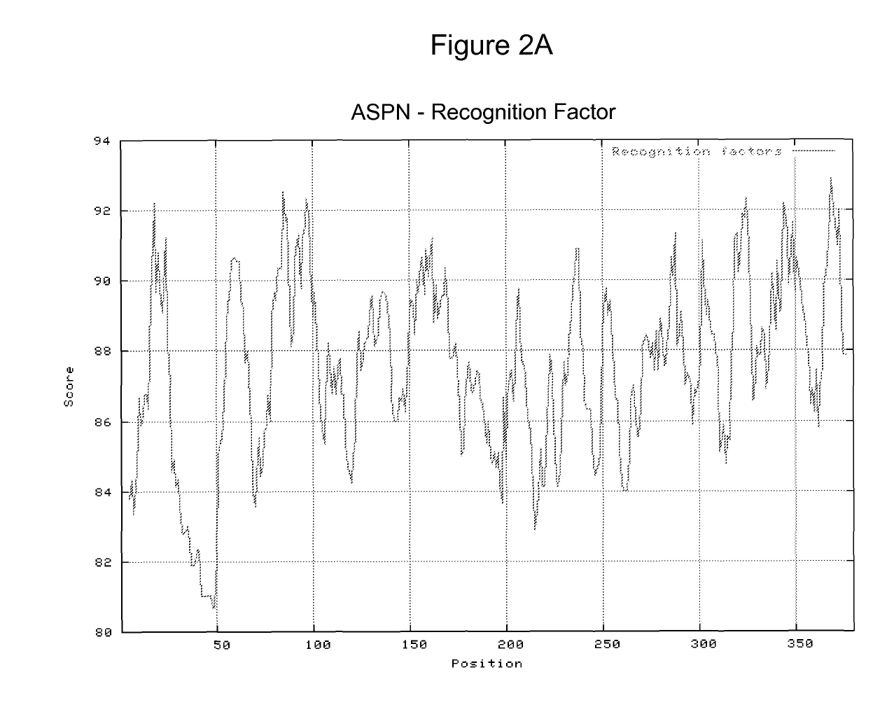

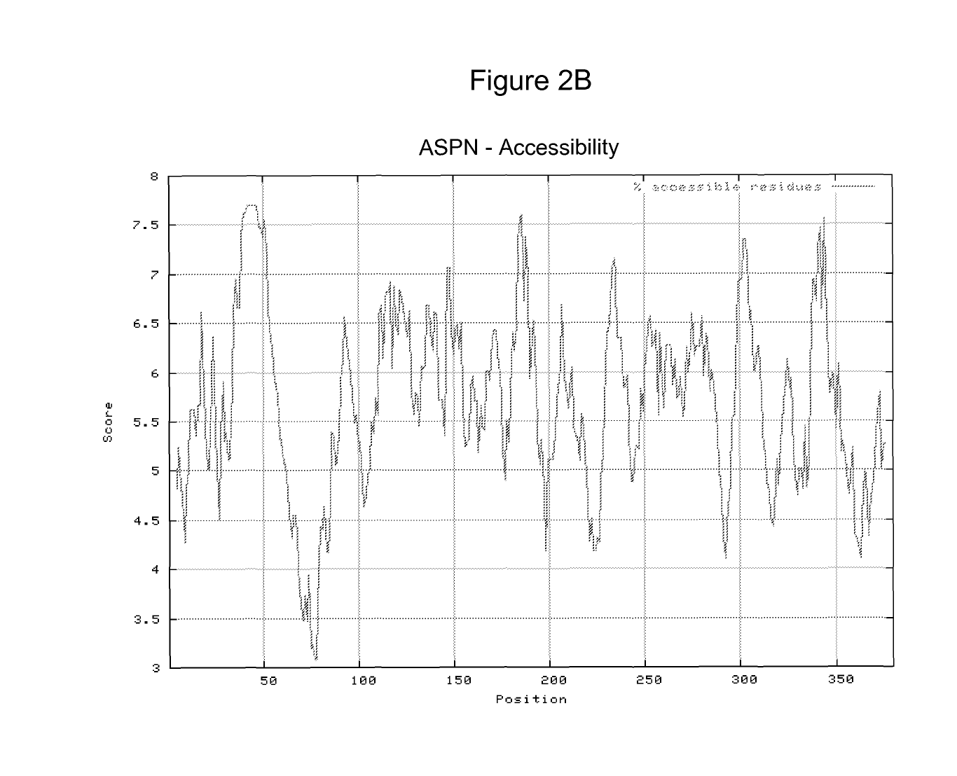

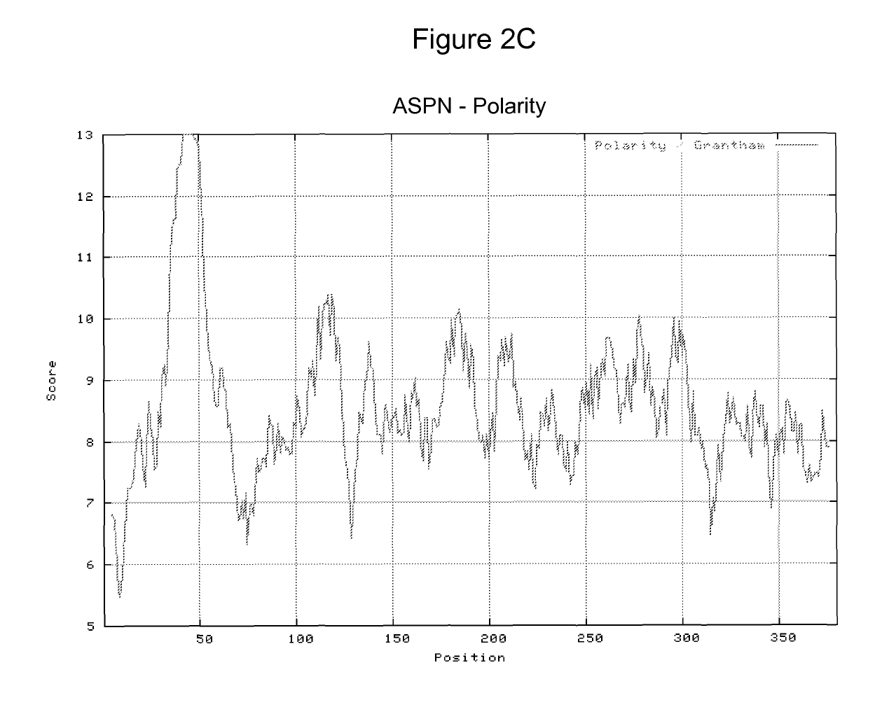

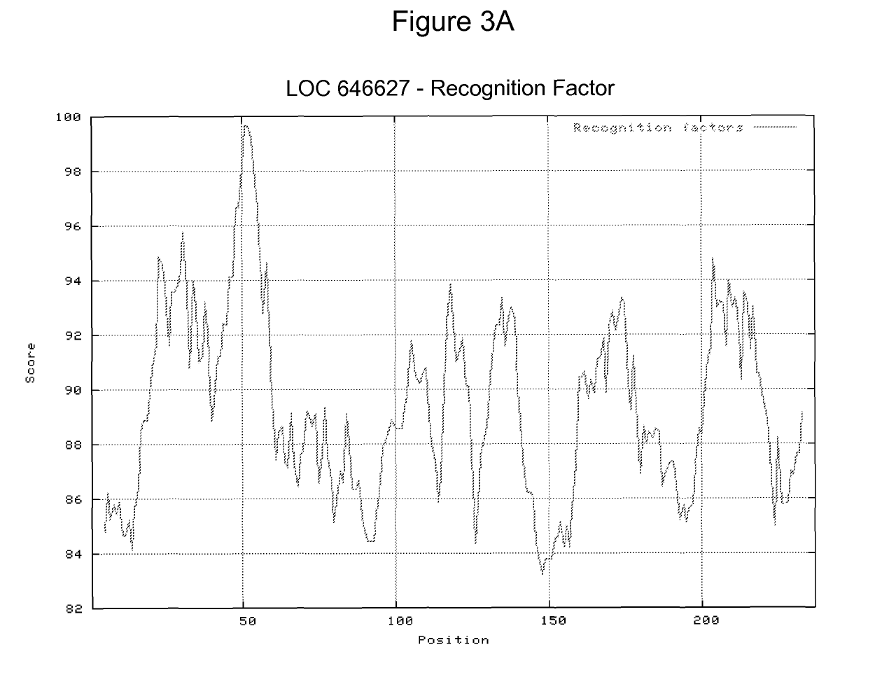

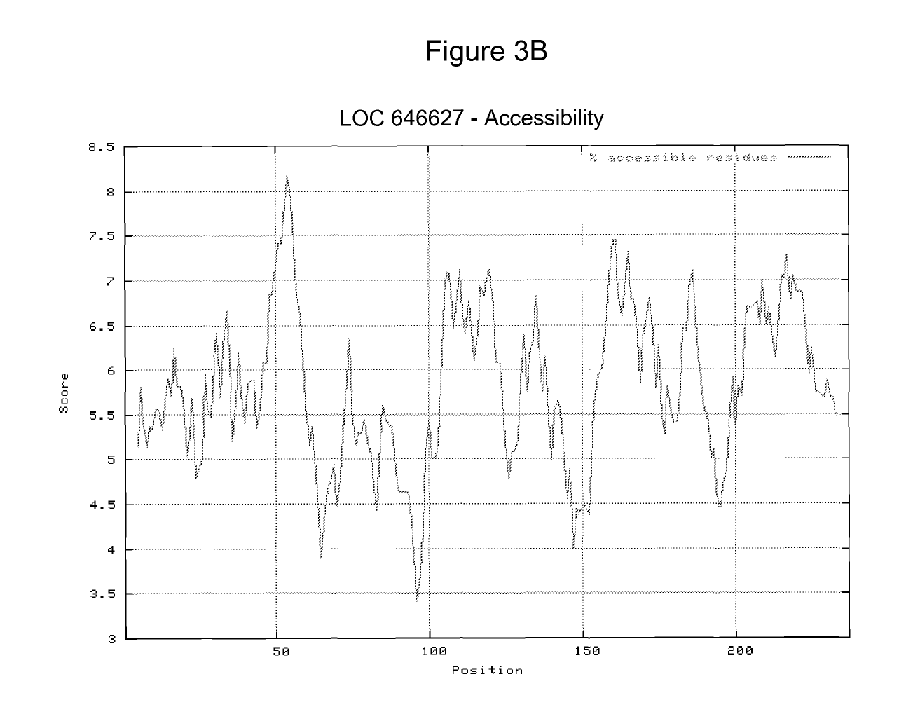

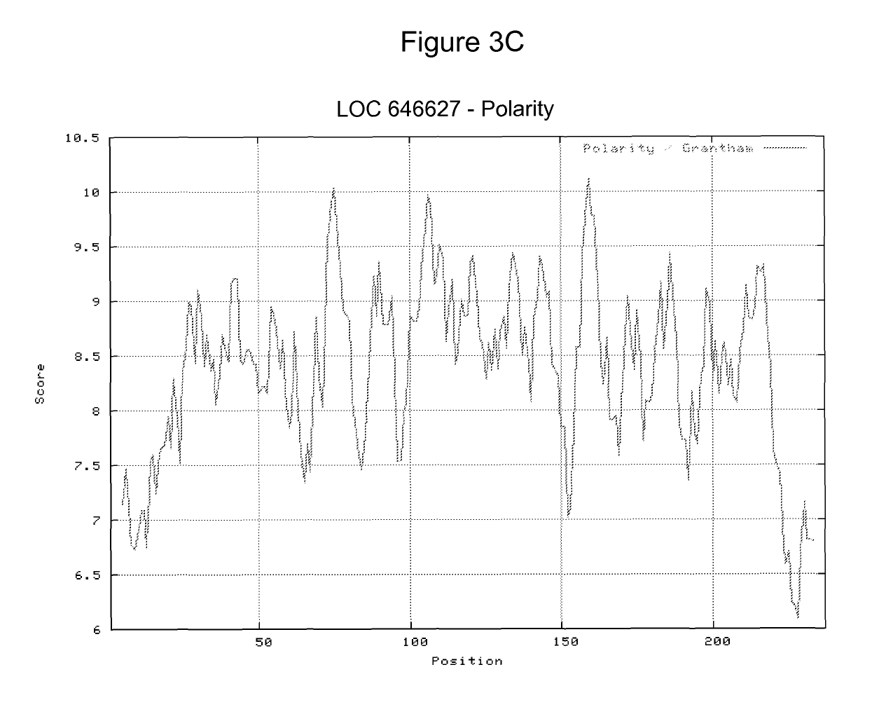

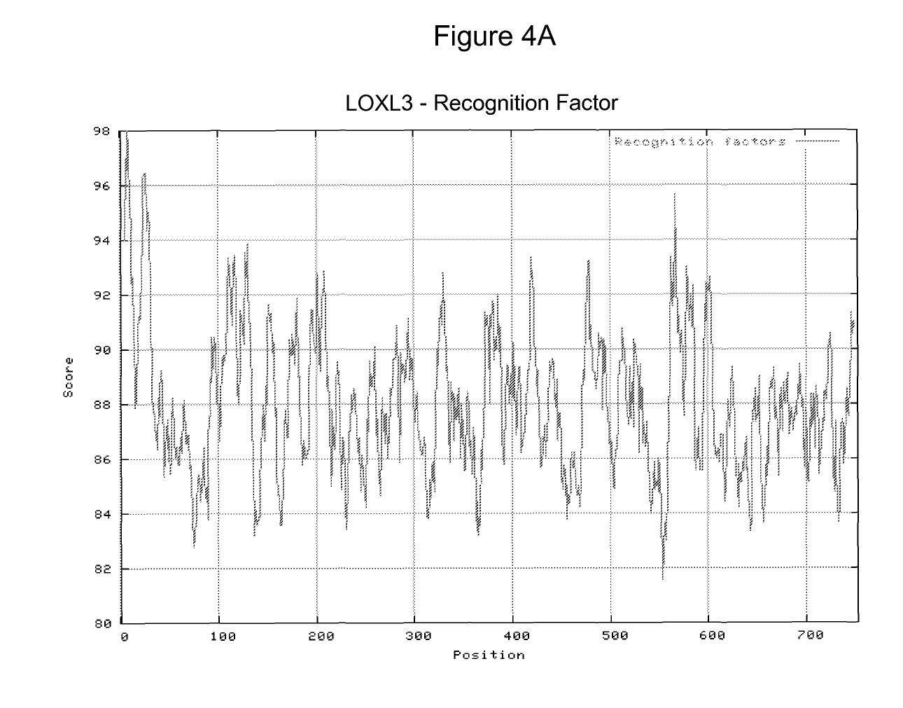

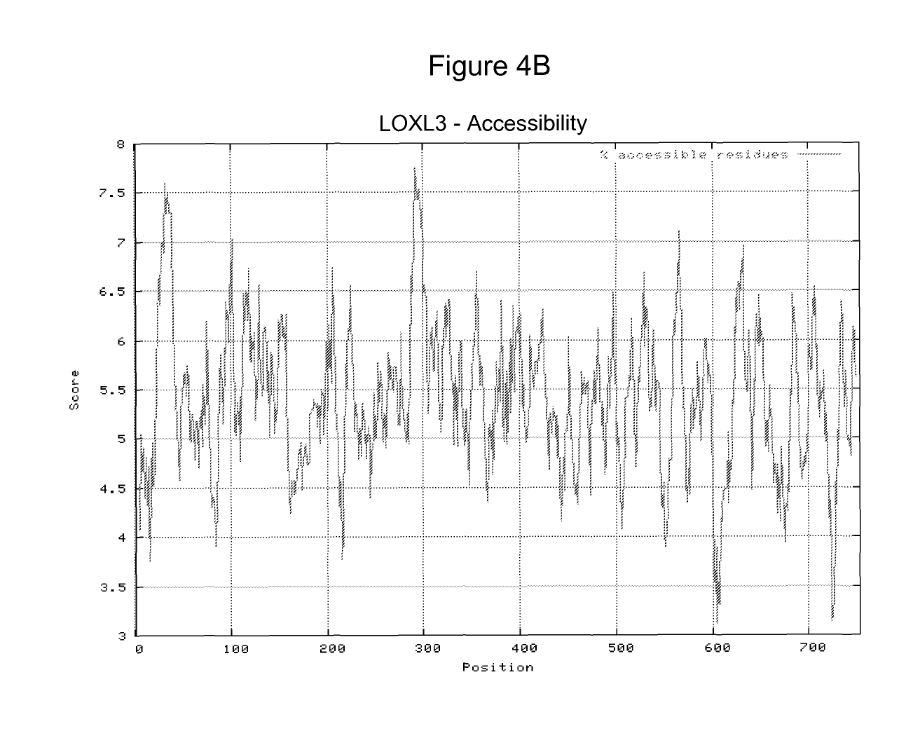

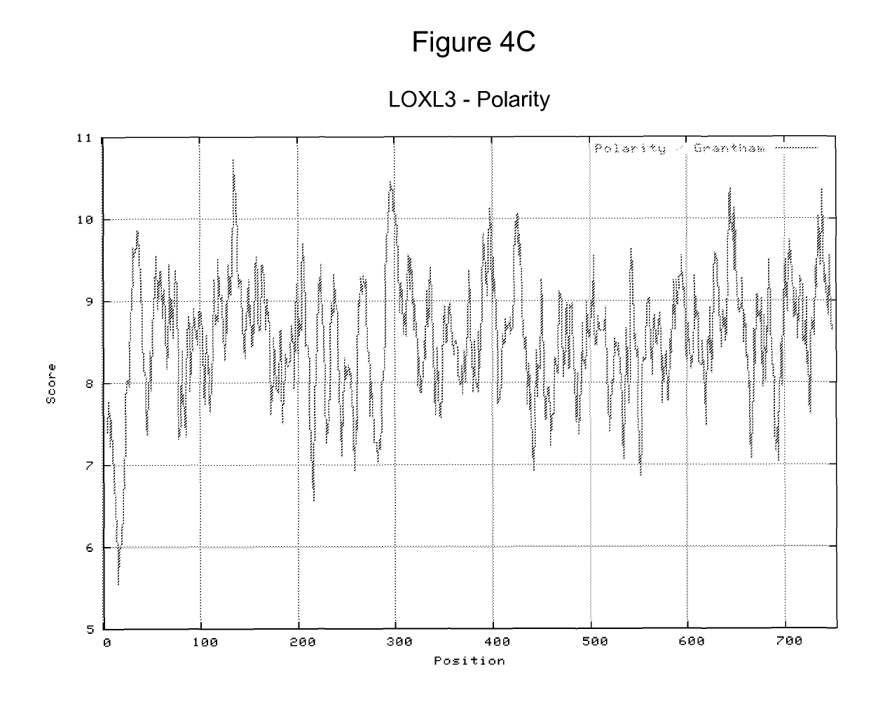

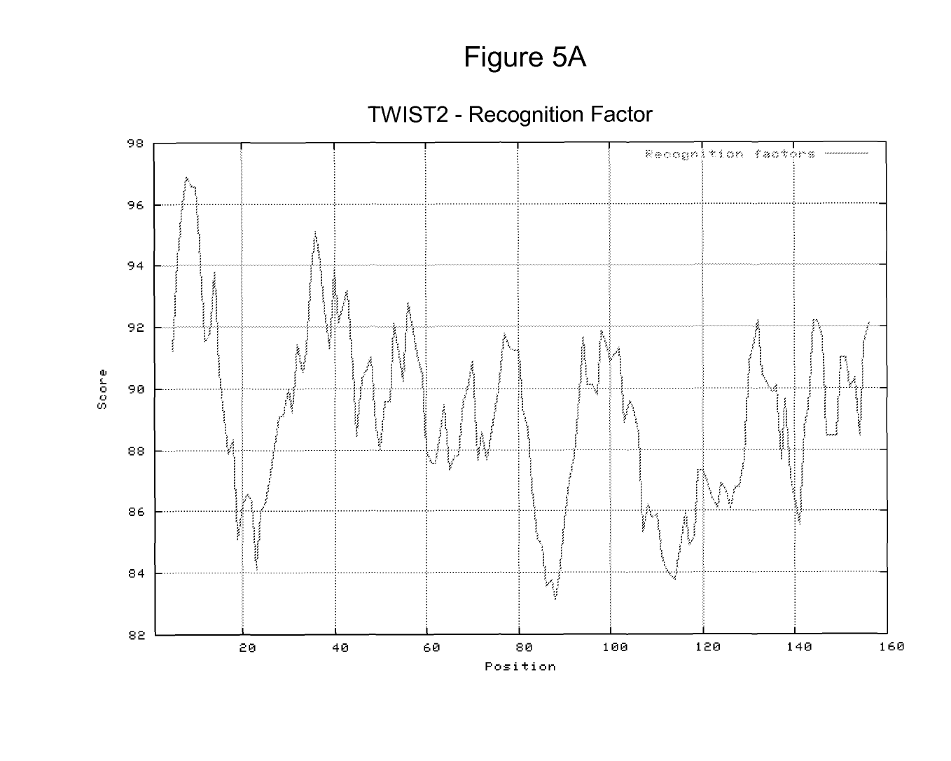

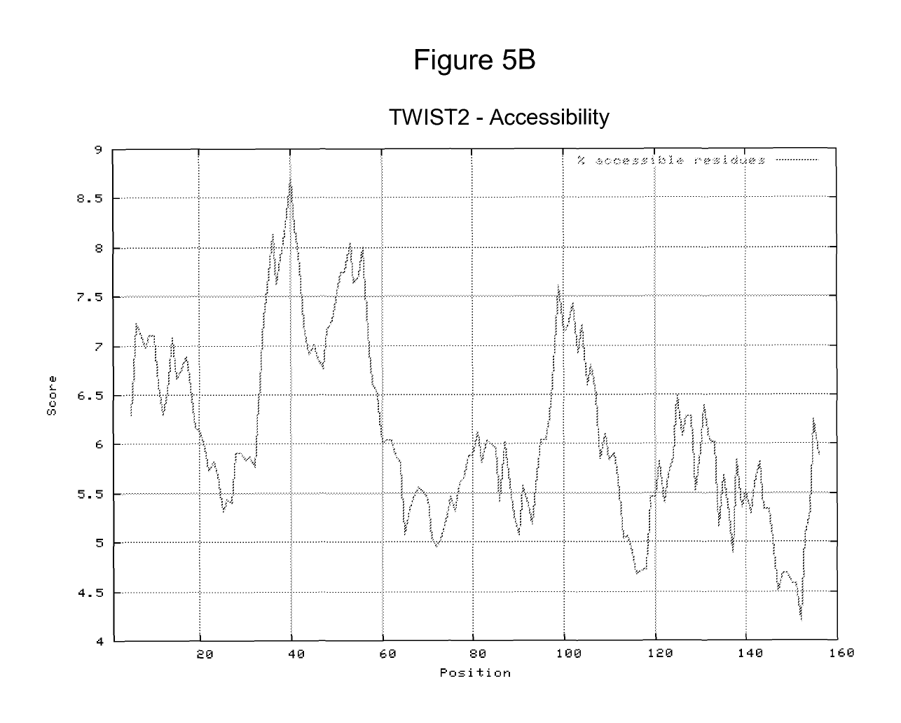

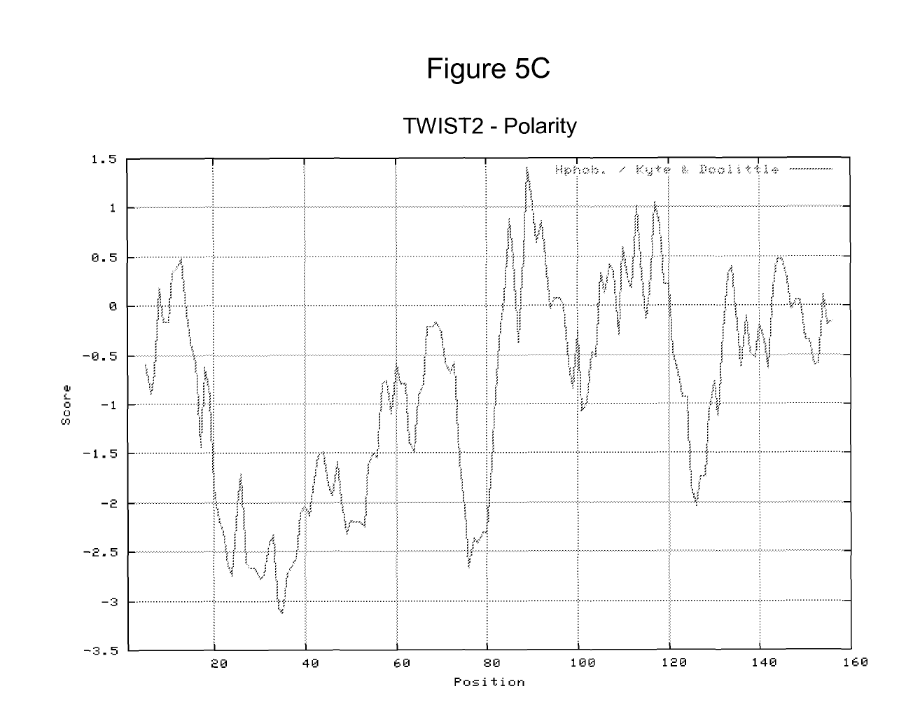

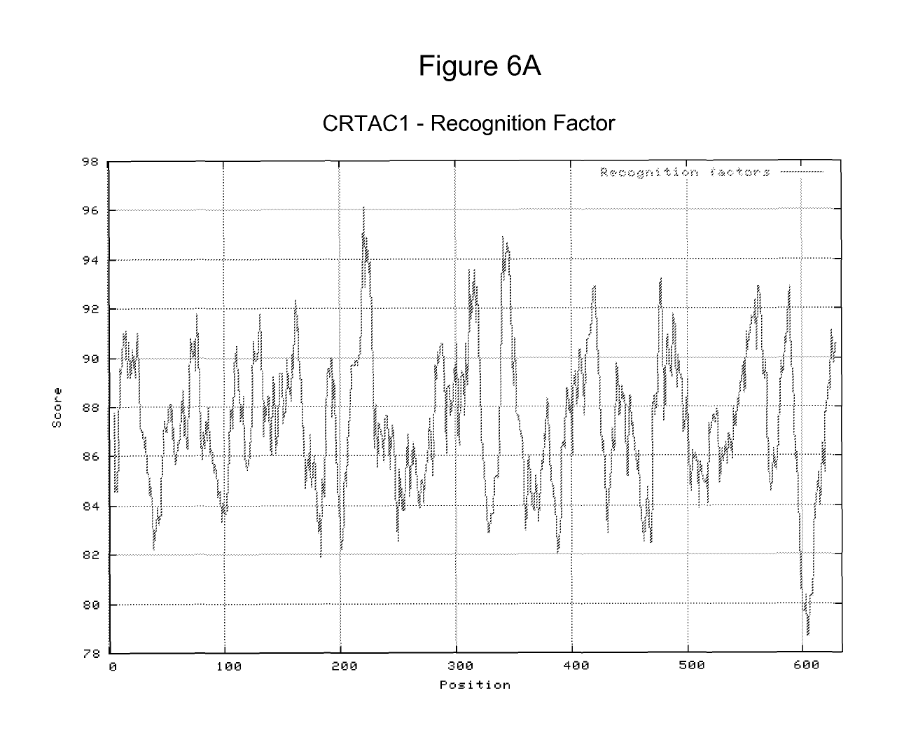

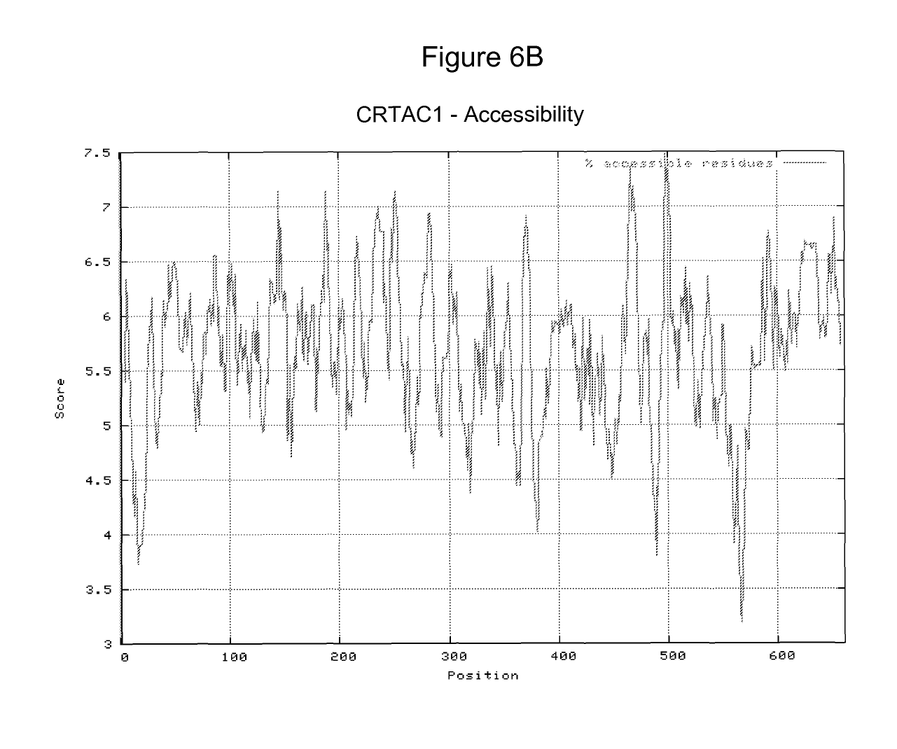

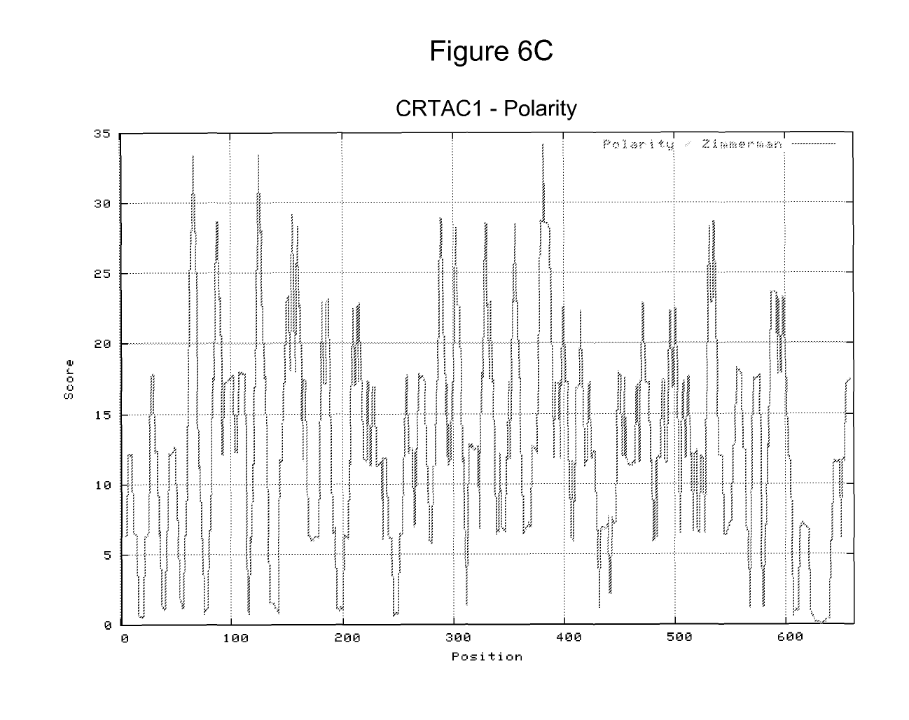

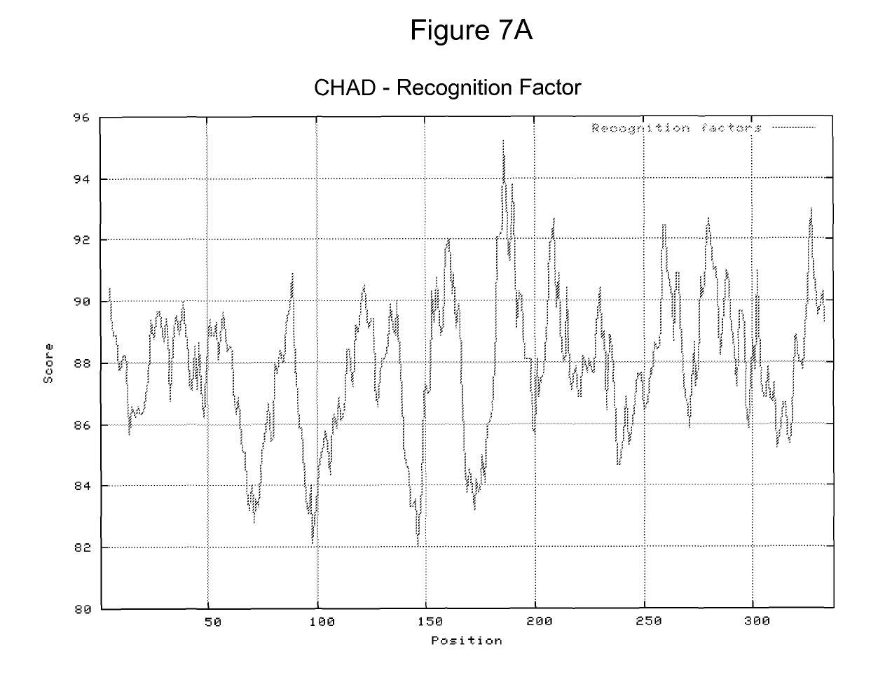

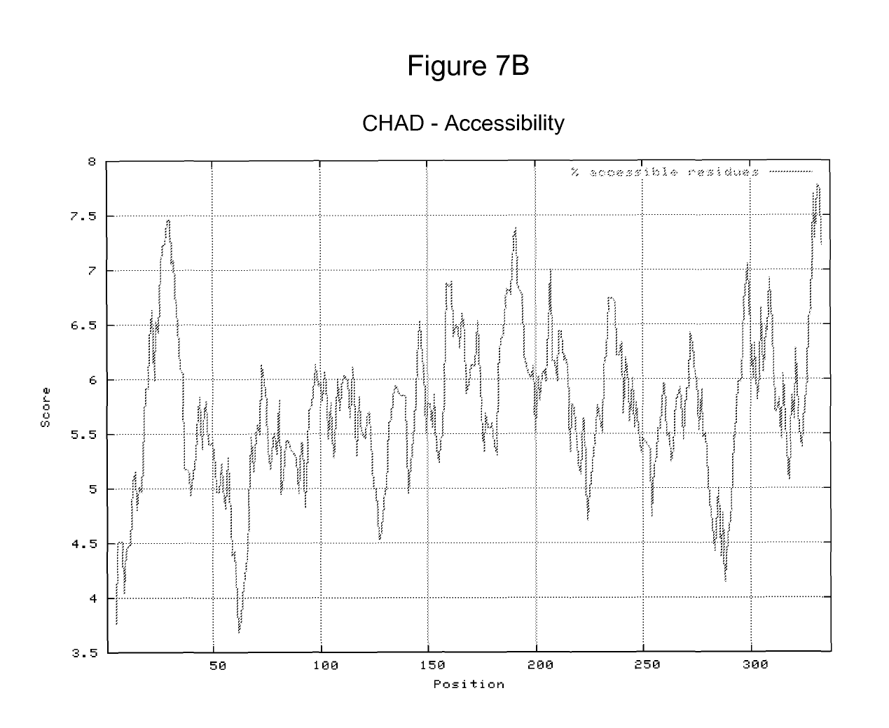

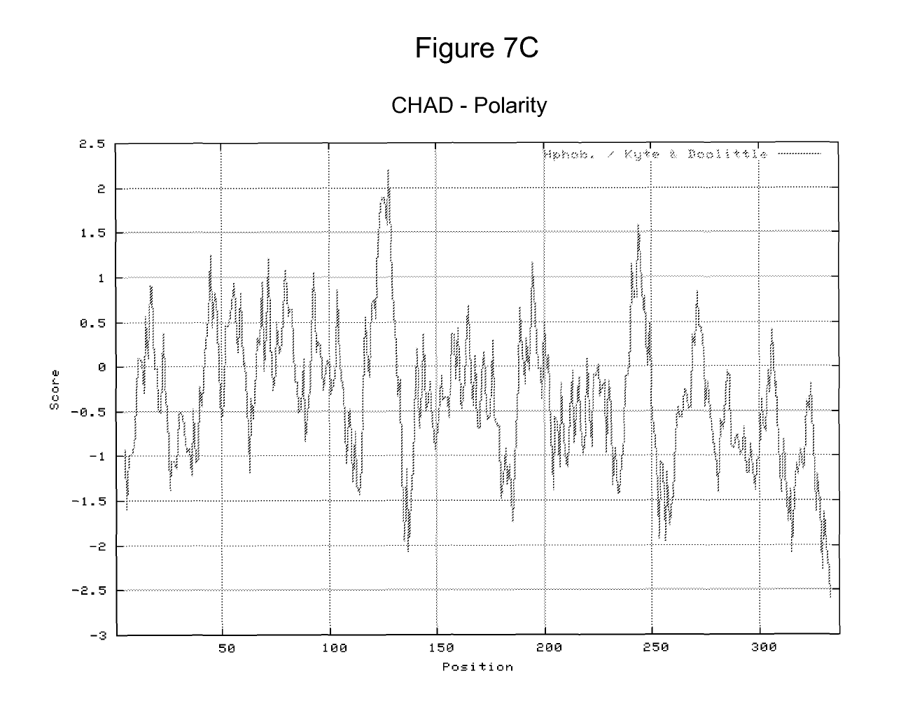

Epitopes were determined by calculation with ProtScale (www.expasy.org/tools/protscale.html) according to the algorithm of Fraga S., “Theoretical prediction of protein antigenic determinants from amino acid sequences,” Can. J. Chem., 60:2606-2610 (1982). The peptide fragments were chosen from those regions of the amino acid sequence of the respective targets, in which a maximum of the epitope recognition factors, accessibility and polarity (corresponding to the results of the ProtScale program) was obtained, since these epitopes proved to be particularly immunogenic and readily accessible for antibodies. The data for the targets: (1) epiphycan (EPYC) (SEQ ID NO: 19) (2) Asporin (ASPN) (SEQ ID NO: 2) (3) LOC 646627 (SEQ ID NO: 28) (4) LOXL3 (SEQ ID NO: 29) (5) TWIST2 (SEQ ID NO: 54) (6) CRTAC1 (SEQ ID NO: 16) and (7) CHAD (SEQ ID NO: 10) are presented below. Recognition factor, accessibility and polarity profiles for each of the aforementioned polypeptides are illustrated in FIGS. 1-7.

Based on the epitope analysis, sequences (epitopes) corresponding to peak maxima of recognition factors, accessibility and polarity were chosen as immunogens for generating anbodies. 2-3 peptide/epitopes were chosen for each of SEQ ID NOs: 1 to 56. Selected epitopes for targets are presented at Table 1:

TABLE 1

| | Epitope 1 | Epitope 2 | Epitope 3 |
|---|---|---|---|
| EPYC | RLIDGSSPQEPEFTGVLGPH | INKNDFASLSDLKRI | FIDISNNRLGRKGIKQEA |
| | (SEQ ID NO: 194) | (SEQ ID NO: 195) | (SEQ ID NO: 196) |
| | (residues 91-110 of | (residues 158-172 of | (residues 215-232 of |
| | SEQ ID NO: 19) | SEQ ID NO: 19) | SEQ ID NO: 19) |
| ASPN | ENKVKKIQKDT | LKKIPSGLPE | KKSLYSAISLF |
| | (SEQ ID NO: 197) (residues | (SEQ ID NO: 198) (residues | (SEQ ID NO: 199) (residues |
| | 180-190 of SEQ ID NO: 2) | 301-310 of SEQ ID NO: 2) | 340-350 of SEQ ID NO: 2) |
| LOC646627 | ISSSASSSLET | NDIESKSLVL | |
| | (SEQ ID NO: 200) (residues | (SEQ ID NO: 201) (residues | |
| | 49-59 of SEQ ID NO: 28) | 158-167 of SEQ ID NO: 28) | |
| LOXL3 | PVYAASSGQKKQQQSK | AAEENCLASSARSANW | ILTPNGTKVAEGHKA |
| | (SEQ ID NO: 202) | (SEQ ID NO: 203) | (SEQ ID NO: 204) |
| | (residues 285-300 of | (residues 555-570 of | (residues 621-635 of |
| | SEQ ID NO: 29) | SEQ ID NO: 29) | SEQ ID NO: 29 |
| TWIST2 | ELERQPKRFGRKRRY | KIIPTLPSDKLSKIQTLKLA | |
| | SKKSSEDGSPTPGKR | (SEQ ID NO: 206) | |
| | (SEQ ID NO: 205) | (residues 91-110 of | |
| | (residues 21-50 of | SEQ ID NO: 54) | |
| | SEQ ID NO: 54) | | |
| CRTAC1 | TGGRGVSVGPILSSSASDIF | VNTYGSYRCRTNKKCSRGYE | AQKRLVNIAVDERSSPYYAL |
| | (SEQ ID NO: 207) | (SEQ ID NO: 208) | (SEQ ID NO: 209) |
| | (residues 238-257 of | (residues 578-597 of | (residues 88-107 of |
| | SEQ ID NO: 16) | SEQ ID NO: 16) | SEQ ID NO: 16) |
| CHAD | DRNQLSSYPSAALSKLRVVE | FRSCKFPTKRSKKAGRH | |
| | (SEQ ID NO: 210) | (SEQ ID NO: 211) | |
| | (residues 203-222 | (residues 343-359 of | |
| | of SEQ ID NO: 10) | SEQ ID NO: 10) | |

The selected peptides were chemically synthesized, conjugated to a suitable carrier protein (KLH, e.g.) and injected into 2 rabbits. For the first immunization, each rabbit received 0.5 mg of the corresponding antigen, mixed with Freund's Adjuvant (EUROGENTEC) and BCG (Baccillus Calmette-Guerin) and 0.25 mg of the immunogens to further increase the immune response. The immunization/bleeding pattern are presented in Table 2.

TABLE 2

| Scheduled Immunizations | | Scheduled Bleeds | |
|---|---|---|---|
| First immunization | Day 0 | Preimmune Bleed | Day 0 |
| Boost No. 1 | Day 7 | Medium Bleed | Day 21 |
| Boost No. 2 | Day 10 | | |
| Boost No. 3 | Day 18 | | |

No terminal bleeding was performed enabling additional boosts and bleeds depending on antibody yields and affinity. Titertests of the antiesera to determine the immune-response were set up as follows: 0.25 μg/ml of the different epitopes were coated on a microtiter plate. The plate was blocked to avoid nonspecific binding. Then the crude serum was diluted with phosphate buffer and 200 μl were incubated on the peptide plate. After the incubation for 3 hours at room temperature the plate was washed and incubated with 200 μl goat anti rabbit-HRP to detect the amount of antibody coated to the peptide of the plate. After this incubation, the plate was washed again and 200 μl TMB was incubated for 20 min. The reaction was stopped with acid and the signal was measured with a reader at 450 nm. Immune response to the target eptiopes was quantified by comparing the signal intensity of the test bleeds at various concentrations with the signal of serum from the same rabbit prior to immunization. Specificity was checked by testing background reaction of the antisera against plates without the epitope under investigation. Table 3 shows the relative signal at an antiserum dilution of 1:5000 of the test bleeds for all epitopes selected:

TABLE 3

| | Rel Titer per Epitope | | |
|---|---|---|---|
| Protein | Epitope 1 | Epitope 2 | Epitope 3 |
| ASPN | 1.62 | x | 1.80 |
| EPYC | 2.12 | 10.70 | x |
| LOC646627 | 1.15 | 1.09 | x |
| LOXL3 | 10.61 | x | 3.25 |
| CHAD | 7.50 | 3.93 | x |
| CRTAC1 | 5.74 | x | 11.99 |
| TWIST2 | 15.96 | 20.08 | x |

Non responding epitopes are marked “X.” All responding antisera were selected for generation of antibody production intended to be used in setting up the screening assays. Antibody preparations from the crude antisera were obtained by affinity purification over standard protein G columns (Protein G-sepharose, 5 ml, GE-Healthcare) using an Akta-Explorer FPLC chromatography system and an internally evaluated standard protocol. The obtained antibody preparations were then used for setting the screening assays for clinical proof of concept.

Clinical evaluation of targets consisted of : (1) setting up a competitive screening assay (2) optimizing the assay sensitivity (3) testing sample pools from individuals with and without a bone/cartilage disease and (4) data analysis and establishing proof of clinical concept by data correlation to the clinical status of the patients and bone/cartilage serum markers.

A competitive assay with the antibody preparations was set up according to the following protocol: The peptide fragments corresponding to the selected epitopes were coated to microtiter plates and blocked to avoid nonspecific binding. Antibody and peptide concentrations were minimized to obtain maximum sensitivity of the assays. The test procedure was as follows: 200 μl of diluted antibody were incubated together with 50 μl of samples overnight. Plates were washed and incubated with 200 μl goat anti rabbit-HRP antibody to detect the amount of antibody on the peptide plate. After the incubation the plate was washed again and incubated with TMB for 20 minutes. The reaction was stopped with diluted sulfuric acid and the signal was measured at 450 nm/630nm with a plate reader.

Pools of samples from patients with osteoporosis and rheumatoid arthritis with a high degree bone (defined by high bone serum markers) or cartilage (defined by the Larson Score, a radiological measure for bone destruction in e.g. rheumatoid joints) degradation were tested. The usage of pool samples rather than of individual samples guarantees data consistency over the whole of the study due to the larger volume available.

Markers for bone and cartilage disease included (1) OPG (osteoprotegerin) and sRANKL (soluble RANK Ligand), markers for osteoclast (bone resorbing cells) regulation (2) DKK-1 (Dickkopf-1) and SOST (Sclerostin), markers for osteoblast (bone forming cells) regulation and (3) SPARC (secreted protein acidic and rich in cysteine), a glycoprotein associated with development, remodeling and tissue repair. The role of these markers in bone and cartilage disease is well documented by a vast number of publications. A correlation of a target to any of these markers, or a correlation to the Larson Score (only for Rheumatoid arthritis) is considered proof for the role of the target in the investigated disease.

The results of the screening tests are summarized in Table 4

TABLE 4

| | | Correlation Coefficient | | | | | |
|---|---|---|---|---|---|---|---|
| | | Larson | | | | | |
| Target | Disease | Score | OPG | DKK-1 | sRANKL | SCST | SPARC |
| EPYC | Osteoporosis | | 0.536 | 0.278 | <0.01 | <0.01 | n/a |
| | Rheumatoid | 0.615 | 0.760 | <0.01 | 0.341 | <0.01 | 0.148 |
| | Arthritis | | | | | | |
| ASPN | Osteoporosis | | <0.01 | 0.526 | 0.838 | 0.306 | n/a |
| | Rheumatoid | 0.726 | <0.01 | 0.294 | 0.091 | 0.165 | 0.940 |
| | Arthritis | | | | | | |
| LOC646627 | Osteoporosis | | <0.01 | <0.01 | 0.679 | <0.01 | n/a |
| | Rheumatoid | 0.783 | 0.835 | <0.01 | 0.348 | <0.01 | <0.01 |
| | Arthritis | | | | | | |
| LOXL3 | Osteoporosis | | 0.949 | 0.216 | <0.01 | <0.01 | n/a |
| | Rheumatoid | 0.865 | 0.824 | <0.01 | 0.327 | <0.01 | <0.01 |
| | Arthritis | | | | | | |
| CHAD | Osteoporosis | | n/a | 0.213 | 0.043 | n/a | n/a |
| | Rheumatoid | 0.100 | 0.26 | <0.01 | 0.706 | n/a | n/a |
| | Arthritis | | | | | | |
| CRTAC1 | Osteoporosis | | n/a | <0.01 | 0.222 | n/a | n/a |
| | Rheumatoid | 0.627 | 0.438 | 0.403 | 0.039 | n/a | n/a |
| | Arthritis | | | | | | |
| TWIST2 | Osteoporosis | | n/a | 0.108 | 0.286 | n/a | n/a |
| | Rheumatoid | 0.319 | <0.01 | <0.01 | n/a | n/a | n/a |
| | Arthritis | | | | | | |

Conclusions with respect to osteoporosis: Epiphycan concentrations showed a moderate (0.536) correlation with OPG. Asporin correlated well with DKK-1 and especially with sRANKL. LOC646627 correlated well with sRANKL. LOXL3 showed the best correlation of all parameters (0.949) to OPG. Based on this data the following targets should be useful markers for osteoporosis: Epiphycan, Asporin, LOC 646627 and LOXL3.

Preferably, a marker is used which shows a correlation coefficient of about 0.5 or higher, more preferably about 0.7 or higher, and most preferably about 0.8 or higher in at least one of the tests as described above.

Conclusions with respect to Rheumatoid Arthritis: Except Chondroadherin, all tested target markers showed a good, albeit broadly ranged (0.319-0.865) correlation to the Larson Score. Interestingly, CHAD did correlate well with sRANKL, which is a very good indicator for disease progression. There was also a surprisingly strong correlation of LOXL3, Epiphycan and LOC646627 with OPG further supporting the concept that these molecules are valuable markers for rheumatoid arthritis. Based on this data, all seven targets are suitable markers for rheumatoid arthritis.

This study is first to demonstrate the generation of antibodies suitable for detecting the markers, the first to demonstrate that the markers circulate in human plasma/serum in detectable amounts and the first to demonstrate that these markers provide useful clinical information about disease status in these patient groups.

1-33. (canceled)

34. A method for diagnosing a bone and/or cartilage disorder in a mammal comprising:
(a) obtaining a test sample from said mammal
(b) contacting the test sample with an antibody or fragment thereof that specifically binds to a polypeptide selected from the group consisting of SEQ ID Nos: 2, 10, 16, 19, 28, 29 and 54;
(c) measuring binding of the antibody or fragment thereof to the test sample; and
(d) comparing binding of step (c) to a normal control level of binding, whereby an alteration in binding of the antibody or fragment thereof to the test sample relative to a normal control level of binding is indicative of a bone and/or cartilage disorder in the mammal

35. The method of claim 34 wherein the antibody or fragment thereof specifically binds to an epitope selected from the group consisting of:
(a) amino acids 180-190 of SEQ ID NO: 2;
(b) amino acids 301-310 of SEQ ID NO: 2;
(c) amino acids 340-350 of SEQ ID NO: 2;
(d) amino acids 203-222 of SEQ ID NO: 10;
(e) amino acids 343-359 of SEQ ID NO: 10;
(f) amino acids 238-257 of SEQ ID NO: 16;
(g) amino acids 578-597 of SEQ ID NO: 16;
(h) amino acids 88-107 of SEQ ID NO: 16;
(i) amino acids 91-110 of SEQ ID NO: 19;
(j) amino acids 158-172 of SEQ ID NO: 19;
(k) amino acids 215-232 of SEQ ID NO: 19;
(l) amino acids 49-59 of SEQ ID NO: 28;
(m) amino acids 158-167 of SEQ ID NO: 28;
(n) amino acids 285-300 of SEQ ID NO: 29;
(o) amino acids 555-570 of SEQ ID NO: 29;
(p) amino acids 621-635 of SEQ ID NO: 29;
(p) amino acids 621-635 of SEQ ID NO: 29;
(q) amino acids 21-50 of SEQ ID NO: 54; and
(r) amino acids 91-110 of SEQ ID NO: 54.

36. The method of claim 34, wherein the antibody or fragment thereof is immobilized on a support substrate selected from at least one of the group of a bead, a slide, a gel, a multi-well plate and a column.

37. The method of claim 34 wherein the mammal is a human.

38. The method of claim 34 wherein the bone and/or cartilage disorder is selected from the group consisting of osteoporosis and rheumatoid arthritis,.

39. The method of claim 34 wherein the normal control level of binding is determined by measuring binding of the antibody or fragment thereof to a sample from one or more undiseased mammals.

40. The method of claim 34, wherein the test sample comprises mammalian body fluids selected form the group consisting of blood, urine, synovial fluid, tears, sweat, saliva, serum, lymph, semen, vaginal fluid, cerebro-spinal fluid, cell culture supernatant, cell extract and tissue extract.

41. The method of claim 34 wherein said antibody is a monoclonal antibody.

42. The method of claim 34 wherein said antibody is a polyclonal antibody.

43. A kit for diagnosing a bone and/or cartilage disorder in a mammal said kit comprising:
(a) an antibody or fragment thereof that specifically binds to a polypeptide selected from the group consisting of SEQ ID Nos: 2, 10, 16, 19, 28, 29 and 54; and
(b) means for detecting binding of said antibody to said polypeptide.

44. The kit of claim 43 wherein the antibody specifically binds to an epitope selected from the group consisting of:
(a) amino acids 180-190 of SEQ ID NO: 2;
(b) amino acids 301-310 of SEQ ID NO: 2;
(c) amino acids 340-350 of SEQ ID NO: 2;
(d) amino acids 203-222 of SEQ ID NO: 10;
(e) amino acids 343-359 of SEQ ID NO: 10;
(f) amino acids 238-257 of SEQ ID NO: 16;
(g) amino acids 578-597 of SEQ ID NO: 16;
(h) amino acids 88-107 of SEQ ID NO: 16;
(i) amino acids 91-110 of SEQ ID NO: 19;
(j) amino acids 158-172 of SEQ ID NO: 19;
(k) amino acids 215-232 of SEQ ID NO: 19;
(l) amino acids 49-59 of SEQ ID NO: 28;
(m) amino acids 158-167 of SEQ ID NO: 28;
(n) amino acids 285-300 of SEQ ID NO: 29;
(o) amino acids 555-570 of SEQ ID NO: 29;
(p) amino acids 621-635 of SEQ ID NO: 29;
(q) amino acids 21-50 of SEQ ID NO: 54; and
(r) amino acids 91-110 of SEQ ID NO: 54.

45. The kit of claim 43, wherein said antibody or fragment thereof is immobilized in a plurality of wells in a multi-well plate.

46. The kit of claim 43, further comprising instructions for use.

47. The kit of claim 43 wherein said antibody is a monoclonal antibody.

48. The kit of claim 43 wherein said antibody is a polyclonal antibody.

49. An isolated antibody generated by immunizing a mammal with a peptide fragment selected from the group consisting of:
(a) amino acids 180-190 of SEQ ID NO: 2;
(b) amino acids 301-310 of SEQ ID NO: 2;
(c) amino acids 340-350 of SEQ ID NO: 2;
(d) amino acids 203-222 of SEQ ID NO: 10;
(e) amino acids 343-359 of SEQ ID NO: 10;
(f) amino acids 238-257 of SEQ ID NO: 16;
(g) amino acids 578-597 of SEQ ID NO: 16;
(h) amino acids 88-107 of SEQ ID NO: 16;
(i) amino acids 91-110 of SEQ ID NO: 19;
(j) amino acids 158-172 of SEQ ID NO: 19;
(k) amino acids 215-232 of SEQ ID NO: 19;
(l) amino acids 49-59 of SEQ ID NO: 28;
(m) amino acids 158-167 of SEQ ID NO: 28;
(n) amino acids 285-300 of SEQ ID NO: 29;
(o) amino acids 555-570 of SEQ ID NO: 29;
(p) amino acids 621-635 of SEQ ID NO: 29;
(q) amino acids 21-50 of SEQ ID NO: 54; and
(r) amino acids 91-110 of SEQ ID NO: 54, wherein said antibody is capable of binding to said peptide fragment.

50. The isolated antibody of claim 49 wherein said antibody is a monoclonal antibody.

51. The isolated antibody of claim 49 wherein said antibody is a polyclonal antibody.
